(19) United States
(12) Patent Application Publication
(10) Pub. No.: US 2017/0327797 A1
(43) Pub. Date: Nov. 16, 2017

(54) RECOMBINANT HBV CCCDNA, THE METHOD TO GENERATE THEREOF AND THE USE THEREOF

(71) Applicant: Hoffmann-La Roche Inc., Little Falls, NJ (US)

(72) Inventors: Lu Gao, Shanghai (CN); Hui Hu, Shanghai (CN); Zhipeng Yan, Shanghai (CN); Kunlun Xiang, Shanghai (CN); Youjun Yu, Shanghai (CN); Jing Zeng, Shanghai (CN)

(73) Assignee: Hoffmann-La Roche Inc., Little Falls, NJ (US)

(21) Appl. No.: 15/661,283

(22) Filed: Jul. 27, 2017

Related U.S. Application Data
(63) Continuation of application No. PCT/EP2016/051409, filed on Jan. 25, 2016.

(30) Foreign Application Priority Data
Jan. 27, 2015 (CN) PCT/CN2015/071604
Sep. 22, 2015 (CN) PCT/CN2015/090258

(51) Int. Cl. C12N 7/00; C07K 14/005; G01N 33/576; C12Q 1/70; G01N 33/50; C12P 19/34; A01K 67/027

(52) U.S. Cl. CPC C12N 7/00; C12P 19/34; C07K 14/005; A01K 67/027; C12Q 1/706; G01N 33/5088; G01N 33/5761; A01K 2227/105; C12N 2730/10151; A01K 2267/0337; C12Q 2600/136; A01K 2207/05; C12N 2730/10121; G01N 2333/02; G01N 2500/10

(57) ABSTRACT

The present invention relates to a recombinant HBV cccDNA comprising HBV genome or the fragment or variant thereof and a site-hybrid insert, a method to generate said recombinant HBV cccDNA, a method for establishment of an in vitro or in vivo cccDNA based model for persistently hepatitis B virus replication by using the recombinant HBV cccDNA of the present invention, and a method for anti-HBV drug evaluation.

### RELATED APPLICATIONS

This application is a continuation of International Application No. PCT/EP2016/051409, filed Jan. 25, 2016 which claims priority to International Application No. PCT/CN2015/071605 filed Jan. 27, 2015 and International Application No. PCT/CN2015/090258, filed Sep. 22, 2015, the contents of which are incorporated herein by reference.

### SEQUENCE LISTING

This application contains a Sequence Listing which has been submitted via EFS-Web and is hereby incorporated by reference in its entirety. Said ASCII copy, created on Jul. 10, 2017, is named Sequence_Listing.txt and is 116,666 bytes in size.

### FIELD OF THE INVENTION

The present invention relates to a recombinant HBV cccDNA comprising HBV genome or the fragment or variant thereof and a site-hybrid insert, a method to generate said recombinant HBV cccDNA, a method for establishment of an in vitro or in vivo cccDNA based model for persistent hepatitis B virus replication by using the recombinant HBV cccDNA of the present invention, and a method for anti-HBV drug evaluation.

### BACKGROUND OF THE INVENTION

Hepatitis B virus (HBV) is one of the most dangerous human pathogens. Although a safe and effective vaccine has been available for longer than two decades, approximately 2 billion people worldwide have been infected with HBV and more than 350 million people are chronically infected (Liaw, et al., 2009, Lancet, 373: 582-92). Chronic Hepatitis B (CHB) infection predisposes to severe liver disease, including liver cirrhosis and hepatocellular carcinoma. HBV infection ranked in the top health priorities in the world, and was the tenth leading cause of death (786 000 deaths per year) according to the 2010 Global Burden of Disease study (Lozano, et al., 2012, Lancet, 380: 2095-128). Current approved drugs have made substantial progresses in treating CHB, however, the cure rate remains lower than 10% (Kwon, et al., 2011, Nat Rev Gastroenterol Hepatol, 8: 275-84).

HBV is a partially double-stranded DNA virus. Upon infection of human hepatocytes, a covalently closed circular DNA (cccDNA) will be formed and maintained in the infected cell nucleus, where it persists as a stable episome and serves as a template for the transcription of all viral genes (Levrero, et al., 2009, J Hepatol, 51: 581-92). The major limitation of current therapy is the failure to eliminate the preexisting cccDNA pool. Therefore, there is an urgent need for development of novel therapeutic agents targeting directly on cccDNA (Fletcher, et al., 2013, Semin Liver Dis, 33: 130-7).

Previous attempts on establishment of HBV cccDNA based in vitro and in vivo models failed to generate satisfactory results. For example, transfection of PCR generated monomeric linear HBV genome could generate cccDNA in hepatocytes (Gunther, et al., 1995, J Virol, 69: 5437-44, Pollicino, et al., 2006, Gastroenterology, 130: 823-37), but the efficiency is low and oligomers may form. To improve this method, the PCR generated monomeric linear HBV genome could be circulated before transfection, but due to the complicated process and low yield, it is very difficult to scale up the DNA production for in vivo studies (Cavallone, et al., 2013, J Virol Methods, 189: 110-7, Qin, et al., 2011, J Clin Microbiol, 49: 1226-33).

Recently, the minicircle technology based on site-specific intramolecular recombination method has been well established, which allows effective production of minicircle DNA with high yield and reproducible high quality (Kobelt, et al., 2013, Mol Biotechnol, 53: 80-9). However, such technology has never been successfully used for the production of recombinant HBV cccDNA.

Therefore, there is a need for an efficient recombinant HBV cccDNA which can be functional for use in the establishment of an in vitro or in vivo cccDNA based model, and a method to efficiently generate large amount of said recombinant HBV cccDNA

Furthermore, anti-HBV drug discovery has been hindered by the lack of convenient and physiological relevant in vitro and in vivo models. Although several in vitro HBV natural infection systems are available, such as primary human hepatocyte (PHH), differentiated HepaRG cells and HepG2 cells with stable NTCP protein expression, performing high throughput screening (HTS) for anti-HBV molecules with these systems are extremely challenging. For example, fresh PHH represents the most physiological relevant in vitro model for HBV drug discovery, but PHH quickly loses its susceptibility to HBV infection upon isolation (Yan, et al., 2012, Elife, 1: e00049). Furthermore, limited supply, high metabolic level and donor to donor variation make this system highly inefficient. HepaRG is the first cell line which could support HBV infection, but the long differentiation and assay time restrict its usage towards HTS (Gripon, et al., 2002, Proc Natl Acad Sci USA, 99: 15655-60). With the discovery of NTCP as HBV entry receptor, genetically engineered HepG2 cell line stably expressing NTCP is susceptible to HBV infection and has quickly become a very useful tool for HBV research and drug discovery(Yan, et al., 2012, Elife, 1: e00049). However, like HepG2.2.15, HepAD38, HepDE19 and HepDES19, all HepG2 derived cell lines have defects in interferon (IFN) mediated anti-HBV response, which is not suitable for identifying and testing IFN pathway related immune modulators (Marozin, et al., 2008, Mol Ther, 16: 1789-97, Keskinen, et al., 1999, Virology, 263: 364-75).

HBV has a very narrow host range, only human, chimpanzee and tree shrew (Tupaia belangeri) are susceptible. None of the genetically and immunologically well-characterized laboratory animals are permissive to HBV infection, which greatly limits not only our research on the mechanisms of HBV immunopathogenesis and persistence, but also anti-HBV drug development. To overcome this limitation, several mouse models have been established by either introducing human hepatocytes to generate chimeric mouse with humanized liver, or introducing HBV DNA into the mouse liver via transgenic, transduction or hydrodynamic injection (HDI) (Dandri, et al., 2014, J Immunol Methods). While chimeric mouse models, such as uPA-SCID mice and FRG mice, support the entire HBV life cycle including entry, cccDNA formation and spreading, they are genetically immune deficient and are not suitable for studying adaptive immune responses (Dandri, et al., 2001, Hepatology, 33: 981-8, Azuma, et al., 2007, Nat Biotechnol, 25: 903-10). Introduction of HBV DNA directly into mouse liver could bypass the entry step, thus allows persistent HBV replication in mouse liver under immunocompetent background. However, the major limitation for these models is that HBV replication is not driven by cccDNA, rendering them less physiologically relevant. Recently, Qi et al. developed an innovative HDI based recombinant cccDNA mouse model, where cccDNA was generated in vivo through Cre/loxP-mediated DNA recombination (Qi, et al., 2014, J Virol, 88: 8045-56). But the viral replication level was low and in vivo persistent time was short (Qi, et al., 2014, J Virol, 88: 8045-56).

In summary, currently existing HBV cell culture models have major limitations. For example, PHH has supply and donor-to-donor variation problems, HepaRG has long differentiation and assay time problem, HepG2-NTCP cell has defects in interferon (IFN) mediated anti-HBV response. Currently existing HBV animal models have major limitations as well. For example, humanized liver chimeric mouse models do not have functional adaptive immunity. In transduction and HDI mouse models, HBV replication is not driven by cccDNA, rendering them less physiologically relevant. In order to improve these limitations, it is necessary to develop cccDNA based models, especially an immunocompetent mouse model that can support cccDNA driven HBV persistent replication, for anti-HBV drug discovery and addressing HBV cccDNA related biological questions.

### SUMMARY OF THE INVENTION

The present invention provides a recombinant HBV cccDNA, and a method to generate recombinant HBV cccDNA. The recombinant HBV cccDNA can contain various nucleotide sequences, such as the HBV genome of any genotype or the fragment or variant thereof. Furthermore, generating recombinant HBV cccDNA in large quantity using this method is also one of the objects of present invention.

When the recombinant HBV cccDNA of the present invention is transfected into cultured cells, it behaves the same as natural HBV cccDNA and it can exist in an episomal form in the cell nucleus supporting HBV replication. With this cell culture model, HBV cccDNA could be conveniently introduced into all primary cells and cell lines by a simple transfection process, bypassing the restriction steps such as entry and cccDNA formation.

When the recombinant HBV cccDNA of the present invention is delivered into a mouse and transfects hepatocytes of the injected mouse, it behaves the same as natural HBV cccDNA and it can exist in an episomal form in the hepatocytes of the mouse for at least 30 days in the hepatocytes, particularly at least 37 days, 44 days or 51 days, and can be used as a HBV transcription template for production of viral antigens, replication intermediates, and mature virions which are released in bloodstream of the injected mouse. The recombinant HBV cccDNA of the present invention can be used for evaluation and elucidation of mechanism of chronic hepatitis and anti-viral drug discovery research.

Furthermore, the present invention also relates to the composition comprising said cccDNA, and the kit comprising said cccDNA, which is useful for establishing an in vitro or in vivo cccDNA based HBV model.

In another embodiment, the present invention also relates to a method to establish an in vitro or in vivo cccDNA based HBV model, which comprises:

(i) generating recombinant HBV cccDNA using minicircle technology; and

(ii) delivering the recombinant HBV cccDNA into a cell line or primary cell or an animal, particularly a mouse.

In another embodiment, the present invention also relates to a use of the recombinant HBV cccDNA of the present invention for establishing an in vitro or in vivo cccDNA based HBV model or for the preparation of a kit or composition used in the method to establish an in vitro or in vivo cccDNA based HBV model.

In a further embodiment, the present invention also relates to the anti-HBV drug evaluation, or the evaluation of a medicament for the treatment of hepatitis B virus infection by the recombinant HBV cccDNA of the present invention.

In a further embodiment, the present invention also relates to a method for anti-HBV drug evaluation, or for evaluating a medicament for the treatment of hepatitis B virus infection.

To be specific, the present invention relates to the following items:
1. A recombinant HBV cccDNA, comprising HBV genome or the fragment or variant thereof and a site-hybrid insert.
2. The recombinant HBV cccDNA of item 1, wherein the site-hybrid insert is attR site.
3. The recombinant HBV cccDNA of item 1 or 2, wherein the attR site is located immediately preceding the starting codon of preS1 gene, and between the terminal protein domain and spacer of the polymerase gene.
4. The recombinant HBV cccDNA of any one of items 1 to 3, wherein the attR site is located between the 2847 and 2848 positions of SEQ ID NO:3.
5. The recombinant HBV cccDNA of anyone of items 1 to 4, wherein the HBV genome is the full length genome, particularly the genotype B or genotype D genome, more particularly the genome specified in GeneBank JN664917.1, X02496, AY217370, AY220698, GQ205440 or HPBHBVAA, most particularly the genome represented by SEQ ID NO:3, SEQ ID NO:22 or SEQ ID NO:23; or is the over length genome, e.g., 1.1 unit or 1.3 unit genome of genotype D (e.g., 1.3 unit genome represented by SEQ ID NO:9).
6. The recombinant HBV cccDNA of any one of items 1 to 5, wherein the fragment of the HBV genome in the recombinant HBV cccDNA can replicate or express the genes encoding envelope proteins, core/precore proteins, x protein and/or polymerase protein of HBV.
7. The recombinant HBV cccDNA of item 1, the sequence of which is listed in SEQ ID NO:2.
8. The recombinant HBV cccDNA of anyone of items 1 to 6, for transfecting a cell line or primary cell.
9. The recombinant HBV cccDNA of item 8, wherein the cell line is the cell line from hepatic cells, particularly those from hepatocyte, more particularly HepG2 or HepaRG, or the primary cell is primary hepatic cells, particularly primary hepatocyte.
10. The recombinant HBV cccDNA of anyone of items 1 to 9, for anti-HBV drug evaluation.
11. The recombinant HBV cccDNA of item 9, wherein the anti-HBV drug is ETV, HAP 12, HAP 2, Pegasys or R848.
12. The recombinant HBV cccDNA of anyone of items 1 toll, for use in the method to establish a cccDNA based HBV animal model, wherein the method comprises delivering said recombinant HBV cccDNA into an animal.
13. The recombinant HBV cccDNA of anyone of claims 1 to 12, wherein the established animal model express HBV antigens for at least 30 days in the hepatocytes, particularly at least 37 days, 42 days, 44 days, 49 days, 51 days, 56 days, 70 days, 104 days, 120 days or 134 days in the hepatocytes.
14. The recombinant HBV cccDNA of anyone of claims 1 to 13, wherein the animal is immunocompetent with functional innate and adaptive immunity.
15. The recombinant HBV cccDNA of anyone of claims 1 to 14, wherein the animal is mouse, particularly the mouse is C3H/HeN or CBA/J mouse.
16. The recombinant HBV cccDNA of anyone of claims 1 to 15, wherein the recombinant HBV cccDNA is delivered into the animal via hydrodynamic injection.
17. The composition or kit comprising the recombinant HBV cccDNA of anyone of claims 1 to 16.
18. A method to prepare recombinant HBV cccDNA of any one of items 1 to 7 comprising the following steps:
   a) HBV genome or the fragment or variant thereof is inserted in and flanked by recombination substrate sites of minicircle DNA producing parental vector to form a parental HBVcircle construct;
   b) the parental HBVcircle construct is transformed into the minicircle producer to generate recombinant HBV cccDNA via site-specific recombination.
19. The method of item 18, wherein minicircle producer is microorganism, preferable bacterium, more particularly Escherichia sp., most particularly E.coli.
20. The method according to item 19, wherein E. coli is strain ZYCY10P3S2T.
21. The method of any one of items 18 to 20, wherein the minicircle DNA producing parental vector contains recombination substrate sites, particularly the recombination substrate sites specific to the recombinase, more particularly specific to integrase, e.g., integrases of ΦC31, R4, TP901-1, ΦBT1, Bxb1, RV-1, AA118, U153, ΦFC1.
22. The method of any one of items 18 to 21, wherein the recombination substrate sites are attP and attB.
23. The method according to any one of items 18 to 22, wherein the minicircle DNA producing parental vector is pMC.CMV-MCS-SV40polyA vector.
24. The method according to any one of items 18 to 23, wherein the DNA sequence of parental HBVcircle construct is listed as SEQ ID NO:1.
25. The method according to any one of items 18 to 24, wherein HBV genome or the fragment or variant thereof is located between the recombination substrate sites.
26. The composition or kit comprising the recombinant HBV cccDNA of anyone of items 1 to 17, which can optionally further contain biocompatible and non-immunogenic solution, such as phosphate buffer solution, saline.
27. The use of recombinant HBV cccDNA according to any one of items 1 to 17 or the composition or kit according to item 26 for transfecting a cell line or primary cell.
28. The recombinant HBV cccDNA of anyone of items 1 to 17 or the composition or kit of item 26, for transfecting a cell line or primary cell.
29. The use of the recombinant HBV cccDNA of any one of items 1 to 17, in the preparation of kit or composition used for transfecting a cell line or primary cell.
30. A method for expressing HBV antigen and DNA in vitro using recombinant HBV cccDNA according to any one of items 1 to 17 or the composition or kit according to item 26, comprising the step of delivering, particularly transfecting said recombinant HBV cccDNA into a cell line or primary cell.
31. A method to establish an in vitro cccDNA based HBV model, comprises:
   (i) generating the recombinant HBV cccDNA of any one of items 1 to 17, or preparing the recombinant HBV cccDNA according to the method of anyone of items 18 to 25;
   (ii) delivering the recombinant HBV cccDNA into a cell line or primary cell.
32. The use according to item 27 or 29, or the recombinant HBV cccDNA or the composition or kit according to item 28, or the method according to item 30 or 31, wherein the cell line is the cell line from hepatic cells, particularly those from hepatocyte, more particularly HepG2 or HepaRG, or the primary cell is primary hepatic cells, particularly primary hepatocyte.
33. The use of the recombinant HBV cccDNA according to any one of items 1 to 17 or composition or kit according to item 26 in evaluation of a medicament for treatment of hepatitis B virus infection, or in anti-HBV drug evaluation.
34. The use of the recombinant HBV cccDNA according to any one of items 1 to 17 in the preparation of a composition or kit for evaluating a medicament for treatment of hepatitis B virus infection, or for anti-HBV drug evaluation.
35. The recombinant HBV cccDNA according to any one of items 1 to 17 or composition or kit according to item 26, for use in evaluation of a medicament for treatment of hepatitis B virus infection, or for anti-HBV drug evaluation.
36. The use according to item 33 or 34, or the recombinant HBV cccDNA or composition or kit according to item 26, wherein the medicament or drug including, but not limited to, nucleoside analogs, HBV capsid inhibitors, interferon or TLR7/8 agonists, for example, ETV, HAP 12, HAP 2, Pegasys or R848.
37. The recombinant HBV cccDNA according to any one of items 1 to 17 or the composition or kit according to item 26, for use in the method to establish a cccDNA based HBV animal model, wherein the method comprises delivering said recombinant HBV cccDNA into an animal.
38. A method for expressing HBV antigen and DNA in vivo using recombinant HBV cccDNA according to any one of items 1 to 17 or the composition or kit according to item 26, comprising the step of delivering recombinant HBV cccDNA into an animal.
39. A method to establish a cccDNA based HBV model, comprises:
   (i) generating the recombinant HBV cccDNA of anyone of items 1 to 17, or preparing the recombinant HBV cccDNA according to the method of anyone of items 18 to 25;
   (ii) delivering the recombinant HBV cccDNA into an animal.
40. The use of the recombinant HBV cccDNA of anyone of items 1 to 17, in the preparation of kit or composition used in the method to establish a cccDNA based HBV animal model, wherein the method comprises delivering said recombinant HBV cccDNA into an animal.
41. The use of the recombinant HBV cccDNA according to any one of items 1 to 17 or the composition or kit according to item 26, for the method to establish a cccDNA based HBV animal model, wherein the method comprises delivering said recombinant HBV cccDNA into an animal.
42. The recombinant HBV cccDNA or the composition or kit according to item 26, or the method according to item 38 or 39, or the use according to item 40 or 41, wherein the established animal model express HBV antigen for at least 30 days in the hepatocytes, particularly at least 37 days, 42 days, 44 days, 49 days, 51 days, 56 days, 70 days, 104 days, 120 days or 134 days in the hepatocytes.
43. The recombinant HBV cccDNA or the composition or kit according to item 26 or 42, or the method according to item 38 or 39 or 42, or the use according to item 40 or 41 or 42, wherein the animal is immunocompetent with functional innate and adaptive immunity.
44. The recombinant HBV cccDNA or the composition or kit according to item 26 or 42 or 43, or the method according to item 38 or 39 or 42 or 43, or the use according to any one of items 40 to 43, wherein the animal is mouse, particularly the mouse is C3H/HeN or CBA/J mouse.
45. The recombinant HBV cccDNA or the composition or kit according to item 26 or 42 or 43 or 44, or the method according to item 38 or 39 or 42 or 43 or 44, or the use according to any one of items 40 to 44, wherein the recombinant HBV cccDNA is delivered into the animal via hydrodynamic injection.

The present invention is further explained in the following embodiment illustration and examples. Those below should not, however, be considered to limit the scope of the invention, it is contemplated that modifications will readily occur to those skilled in the art, which modifications will be within the spirit of the invention and the scope of the appended claims.

### BRIEF DESCRIPTION OF THE DRAWINGS

FIG. 1 shows HBVcircle construct design and production. (A) Process of generating HBVcircle with minicircle technology. HBV sequences flanked by attB and attP sites were cloned into the minicircle parental plasmid vector. The recombinant parental HBVcircle construct was then transformed into the minicircle producer E. coli strain ZYCY10P3S2T. Upon the expression of ΦC31 integrase and I-SceI homing endonuclease by adding arabinose, ΦC31 integrase catalyzed recombination between attB and attP sites, leading to the generation of HBVcircle carrying a small attR site, as well as plasmid backbone circle. I-SceI homing endonuclease initiated the destruction of the parental unrecombinated DNA as well as the plasmid backbone circle by digesting the I-SceI recognition sites. HBVcircle DNA was then extracted from the minicircle producer E. coli. (B) Design of HBVcircle. The sequence of attR site is located between 2847 and 2848 positions immediate preceding the starting codon of preS1 gene, as well as between the TP domain and spacer of the polymerase gene. (C) Design and production of HBVcircle-CMV-HBV1.1, HBVcircle-HBV1.3 and HBVcircle. The design of these three HBVcircle constructs was illustrated in upper panel. After minicircle production, the parental DNA and minicircle DNA were linearized by restriction enzyme digestion and electrophoresis analysis was performed.

FIG. 2 shows HBVcircle supports high level HBV replication in transfected cells. Parental and HBVcircle DNA were transiently transfected into HepG2 cells and supernatant was measured for (A) HBeAg (B) HBsAg and (C) HBV DNA using ELISA and qRT-PCR. (D) Cells were lysed, cccDNA was extracted and quantified using RT-PCR. (E) After HBVcircle DNA transfection, HepG2 cells were fixed and stained with anti-HBsAg and anti-HBeAg antibodies for immunofluorecent analysis. Cell nucleuses were visualized with DAPI (4′,6-diamidino-2-phenylindole) staining.

FIG. 3-1 shows characterization of HBVcircle wildtype and HBc(−) mutant in vitro. Wildtype or mutant HBVcircle DNA in the presence or absence of HBc expressing plasmid was transiently transfected into HepG2 cells, and supernatant measured for (A) HBsAg and (B) HBeAg quantification using ELISA. (C) Cell were lysed and cell lysates were subjected to southern blot analysis for encapsidated HBV DNA detection, as well as western blot analysis for HBV capsid, HBc and beta-actin detection with specific antibodies.

FIG. 3-2 shows characterization of HBVcircle wildtype and mutans in vitro. Wildtype or mutant HBVcircle DNA was transiently transfected into HepG2 cells. Supernatant measured for (A) HBsAg and (B) HBeAg quantification using ELSA. (C) Cells were lysed and cell lysates were subjected to western blot analysis for HBV capsid, HBc, HBs and beta-actin detection with specific antibodies.

FIG. 4 shows that HBVcircle is a surrogate for the natural HBV cccDNA. (A) Parental and HBVcircle DNA were transiently transfected into HepG2 cells, cells were lysed and cccDNA was prepared by Hirt method and detected by southern blot. (B) cccDNA or (C) RL30 associated histone H3, H3K9me3 and H3K27ac from HBVcircle transfected HepG2 cells were detected by CHIP.

FIG. 5 shows in vitro anti-HBV drug evaluation with HBVcircle. (A) HepG2 cells were firstly transfected with HBVcircle and then treated with indicated concentrations of ETV or HAP 12 for 6 days. Supernatants were collected and HBsAg, HBeAg and albumin ELISA were performed. Cell were lysed and cell lysates were subjected to southern blot analysis for encapsidated HBV DNA detection, as well as western blot analysis for HBV capsid, HBc and beta-actin detection with specific antibodies. (B) Proliferating HepaRG cells were transfected with HBVcircle and treated with different concentrations of Pegasys for 6 days as above. Supernatants were collected and HBsAg, HBeAg and albumin ELISA were performed.

FIG. 6 shows establishment of persistent HDI mouse model with HBVcircle. Indicated DNA constructs were hydrodynamically injected into the tail vain of C3H/HeN mice. At indicated time points post HDI, blood samples were collected for HBV markers testing, including (A) HBsAg, (B) HBeAg and (C) HBV DNA. (D) Mice body weight was measured at indicated time points.

FIG. 7 shows cccDNA driven HBV persistency in vivo. Different amount of HBVcircle DNA or 10 μg pBR322-HBV1.3 DNA was hydrodynamically injected into C3H/HeN mice. The mice were monitored for 51 days and at indicated time points, serum samples were collected and tested for HBV markers including (A) HBsAg, (B) HBeAg and (C) HBV DNA. (D) On day 3 and day 30, 2 mice each time from the HBVcircle 10 μg group were randomly selected and sacrificed. The cccDNA in mouse livers were detected by southern blot.

FIG. 8 shows cccDNA driven HBV persistency in vivo by liver IHC staining. On day 120 after HDI injection, liver sections from the indicated mice were stained with anti-HBc antibody. Solid arrows show HBc-positive staining cells, empty arrows show HBc-negative staining cells.

FIG. 9 shows in vivo anti-HBV drug efficacy evaluation. (A) On day 0, 10 μg HBVcircle was hydrodynamically injected into C3H/HeN mice. Mice were grouped based on day 21 serum HBsAg levels, and antiviral compound treatment was given orally starting from day 23 to day 51 after HDI. At indicated time points, serum samples were collected and tested for HBV markers including (A) HBsAg, (B) HBeAg and (C) HBV DNA.

FIG. 10 shows establishment of persistent HDI mouse model in CBA/J mouse. Indicated DNA constructs were hydrodynamically injected into the tail vain of CBA/J mice. At indicated time points post HDI, blood samples were collected for HBV markers testing, including (A) HBsAg, (B) HBeAg and (C) HBV DNA. (D) Mice body weight was measured at indicated time points. (E) Percentage of HBsAg positive mice was plotted in according to the serum HBsAg testing results.

FIG. 11 shows establishment of persistent HDI mouse model using HBVcircle with other genotype sequences. Indicated DNA constructs were hydrodynamically injected into the tail vain of C3H/HeN mice. At indicated time points post HDI, blood samples were collected for HBV markers testing, including (A) HBsAg, (B) HBeAg and (C) HBV DNA. (D) Mice body weight was measured at indicated time points.

FIG. 12 shows evaluation of HBV replication in vivo using HBVcircle mutants. Indicated DNA constructs were hydrodynamically injected into the tail vain of C3H/HeN mice. At indicated time points post HDI, blood samples were collected for HBV markers testing, including (A) HBsAg, (B) HBeAg and (C) HBV DNA. (D) Mice body weight was measured at indicated time points.

FIG. 13 shows evaluation of HBV replication in vivo by liver IHC staining. (A)On day 56 after HDI injection, liver sections from the indicated mice were stained with anti-HBc antibody. Solid arrows show HBc-positive staining cells, empty arrows show HBc-negative staining cells. (B) Accumulated staining scores from Table 4 were plotted.

FIG. 14 shows evaluation of HBV replication in vivo using HBVcircle mutants. Indicated DNA constructs were hydrodynamically injected into the tail vain of C3H/HeN mice. At indicated time points post HDI, blood samples were collected for HBV markers testing, including (A) HBsAg, (B) HBeAg and (C) HBV DNA. (D) Mice body weight was measured at indicated time points. (E) Percentage of HBsAg positive mice was plotted in according to the serum HBsAg testing results. (F) Individual HBsAg levels were plotted for wildtype group mice and HBe(−) mutant group.

### DEFINITIONS

Unless defined otherwise, all technical and scientific terms used herein have the same meaning as commonly understood by one of ordinary skill in the art to which this invention pertains.

Although essentially any methods and materials similar to those described herein can be used in the practice or testing of the present invention, only exemplary methods and materials are described. For purposes of the present invention, the following terms are defined below.

As used herein, “hepatitis B virus” or “HBV” refers to a member of the Hepadnaviridae family having a small double-stranded DNA genome of approximately 3,200 base pairs and a tropism for liver cells. “HBV” includes hepatitis B virus that infects any of a variety of mammalian (e.g., human, non-human primate, etc.) and avian (duck, etc.) hosts. “HBV” includes any known HBV genotype, e.g., serotype A, B, C, D, E, F, and G; any HBV serotype or HBV subtype; any HBV isolate; HBV variants, e.g., HBeAg-negative variants, drug-resistant HBV variants (e.g., lamivudine-resistant variants; adefovir-resistant mutants; tenofovir-resistant mutants; entecavir-resistant mutants; etc.); and the like.

As used herein, the “HBV genome” not only refers to the full length genome (1 unit genome), but also to the more than full length HBV genome (>1 unit genome, in other words, over length HBV genome). HBV genome contains all of the information needed to build and maintain HBV replication. Such genome sequences are available in articles and in GeneBank for each genotype. A “more than full length HBV genome” refers to a sequence which comprises a full length genome plus a part of the genome. The sequence of the “more than full length HBV genome” varies based on the desired genome unit and the specific HBV strains. Moreover, the method to obtain the more than full length HBV genome and to determine the sequence of said genome is described in the prior art document, e.g., in European Patent EP1543168.

As used herein, the “fragment of the HBV genome” or the “HBV genomic fragment” can be used interchangeably, and refers to a part of HBV genome. The fragment can be at least 100, 200, 300, 400, 500, 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900, 3000, 3100 or 3200 continuous nucleotides of the HBV genome. The fragment can also be the partial genome containing one or more of the gene contained in the HBV genome, e.g., the fragment can be the nucleic acids encoding envelope proteins, core/precore proteins, x protein and/or polymerase protein of HBV. Moreover, the fragment can be the nucleic acids encoding one or more parts of envelope proteins, core/precore proteins, x protein and/or polymerase protein of HBV.

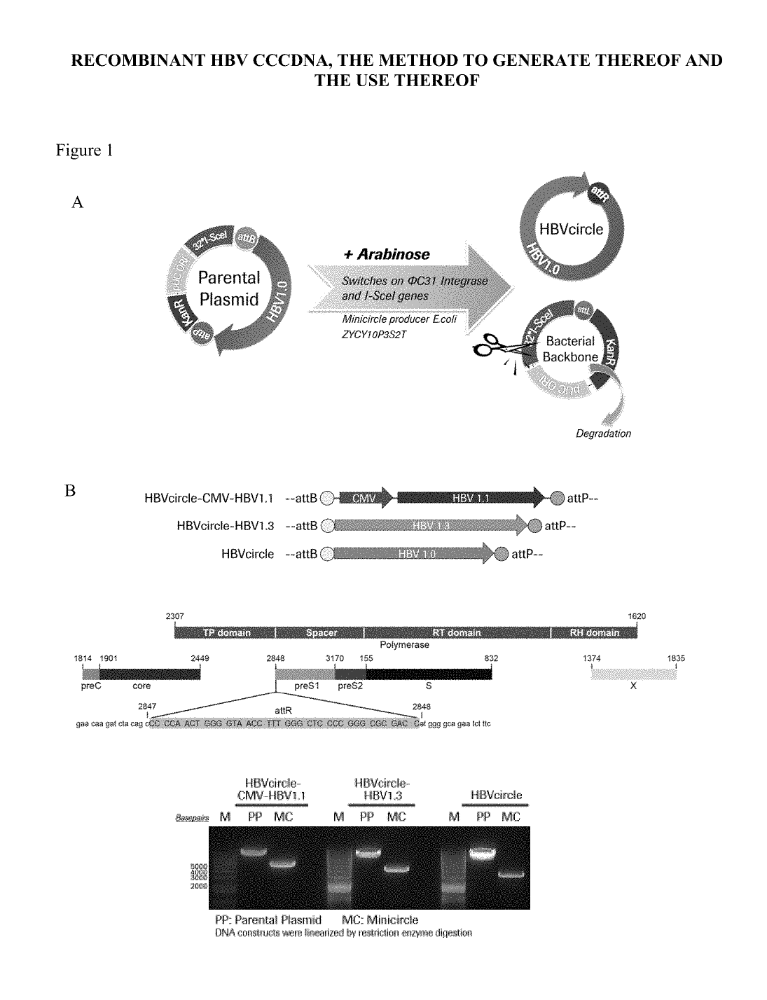

As used herein, when referring to the position of the HBV genome, the numbering of nucleotides is by reference to the whole HBV genomic DNA sequence published in H. Norder et al. (Virology 1994, 198, 489-503 , incorporated herein by reference; FIG. 1 of this article presents an alignment of the genome of various HBV clones representing genotypes C, E and F, with the sequence of clone pHBV-3200 which is 3221 nucleotides long) or the one of a genotype D HBV published in GeneBank under access number JN664917.1 (incorporated herein by reference). The length of the genome of the various HBV is variable. That is to say that numbering of nucleotides should only be considered as illustrative embodiments.

As used herein, the term “variant” or “mutant” can be used interchangeably and is used in reference to polypeptides or polynucleotides that have some degree of amino acid/nucleotide sequence identity to a parent polypeptide sequence or polynucleotides. A variant is similar to a parent sequence, but has at least one or several or more substitution(s), deletion(s) or insertion(s) in their amino acid sequence or nucleotide sequence that makes them different in sequence from a parent polypeptide or parent polynucleotide. In some cases, variants have been manipulated and/or engineered to include at least one substitution, deletion, or insertion in their amino acid sequence or nucleotide sequence that makes them different in sequence from a parent. Additionally, a variant may retain the functional characteristics or activity of the parent polypeptide, or the parent polynucleotide, e.g. , maintaining a biological activity that is at least 50%, 60%, 70%, 80%, 90%, 95%, 98%, or 99% of that of the parent polypeptide or parent polynucleotide

As used herein, the term “nucleic acid construct” refers to a nucleic acid sequence that has been constructed to comprise one or more functional units not found together in nature. Examples include circular, linear, double-stranded, extrachromosomal DNA molecules (plasmids), cosmids (plasmids containing COS sequences from lambda phage), viral genomes comprising non-native nucleic acid sequences, and the like.

As used herein, the term “vector” refers to a vehicle capable of transferring nucleic acid sequences to target cells. For example, a vector may comprise a coding sequence capable of being expressed in a target cell. For the purposes of the present invention, “vector construct” generally refers to any nucleic acid construct capable of directing the expression of a gene of interest and which is useful in transferring the gene of interest into target cells. Thus, the term includes cloning and expression vehicles, as well as integrating vectors.

A “minicircle vector”, or a “minicircle DNA producing parental vector”, as used interchangeably herein, refers to a small, double stranded circular DNA molecule that provides for persistent, high level expression of a sequence of interest that is to be introduced into the vector, which sequence of interest may encode a polypeptide, an shRNA, an anti-sense RNA, an siRNA, and the like in a manner that is at least substantially expression cassette sequence and direction independent. The sequence of interest is operably linked to regulatory sequences present on the minicircle vector, which regulatory sequences control its expression. Such minicircle vectors are described, for example in published U.S. Patent Application US20040214329, herein specifically incorporated by reference.

The overall length of the subject minicircle vectors is sufficient to include the desired elements as described below, but not so long as to prevent or substantially inhibit to an unacceptable level the ability of the vector to enter the target cell upon contact with the cell, e.g., via system administration to the host comprising the cell. As such, the minicircle vector is generally at least about 0.3 kb long, often at least about 1.0 kb long, where the vector may be as long as 10 kb or longer, but in certain embodiments do not exceed this length.

Minicircle vectors differ from bacterial plasmid vectors in that they lack an origin of replication, and lack drug selection markers commonly found in bacterial plasmids, e.g. β-lactamase, tet, and the like. Also expression silencing sequences are found absent, for example, in plasmid backbones, e.g. the parental plasmid backbone nucleic acid sequences from which the minicircle vectors are excised. The minicircle may be substantially free of vector sequences other than the recombinase hybrid product sequence, and the sequence of interest, i.e. a transcribed sequence and regulatory sequences required for expression.

The minicircle vectors comprise a site-hybrid sequence (also known as product hybrid sequence) of a unidirectional site-specific recombinase. As used herein, the “site-hybrid sequence” or “site-hybrid insert” or “product hybrid sequence” can be used interchangeably, and is the result of a unidirectional site specific recombinase mediated recombination of two recombination substrate sites as they are known in the art, e.g., attB and attP substrate sequences (Smith et al., Nucleic Acid Research, 2004, 33:8:2607-2617), and may be either the attR or attL site-hybrid sequence. The “site-hybrid sequence” can be determined by the skilled person according to the recombinase used. Typically, the site-hybrid sequence ranges in length from about 10 to about 30 bp, 40 bp, 50 bp, 60 bp, 70 bp, 80 bp, 90 bp, 100 bp, 150 bp, 200 bp, 250 bp, 300 bp, 350 bp, 400 bp, 450 bp and 500 bp The “recombinase” used herein is a genetic recombination enzyme, which usually derived from bacteria and fungi and catalyze directionally sensitive DNA exchange reactions between short (30-40 nucleotides) target site sequences that are specific to each recombinase. The examples of the recombinase include, but not limited to integrase, e.g., wild-type phage integrases or mutants thereof, where specific representative integrases of interest include, but not limited to, the integrases of ΦC31, R4, TP901-1, ΦBT1, Bxb1, RV-1, AA118, U153, ΦFC1, and the like.

As used herein, the term “recombinant” DNA molecules refers to DNA molecules formed by laboratory methods of genetic recombination (such as molecular cloning) to bring together genetic material from multiple sources, creating sequences that would not otherwise be found in biological organisms. Recombinant DNA is possible because DNA molecules from all organisms share the same chemical structure. They differ only in the nucleotide sequence within that identical overall structure.

The term “site-specific recombination” used herein refers to recombination between two nucleotide sequences that each comprises at least one recognition site. “Site-specific” means at a particular nucleotide sequence, which can be in a specific location in the genome of a host cell. The nucleotide sequence can be endogenous to the host cell, either in its natural location in the host genome or at some other location in the genome, or it can be a heterologous nucleotide sequence, which has been previously inserted into the genome of the host cell by any of a variety of known methods.

A “minicircle producer” as used herein, refers to microorganisms which allow amplification of minicircle DNA producing parental vector, as well as generation of minicircle DNA upon the expression of recombinase. The known minicircle producer in the prior art includes the bacterium, e.g., Escherichia sp., e.g., E. coli. One illustrative example of the minicircle producer in the art is strain ZYCY10P3 S2T.

An “HBVcircle” or “recombinant HBV cccDNA”, as used herein, refers to a minicircle vector comprising HBV genome or the fragment or variant thereof.

Methods of delivering the recombinant HBV cccDNA into a cell are known in the art. For example, the recombinant cccDNA can be delivered into a cell by transfection. Methods of transfecting cells are well known in the art. By “transfected” it is meant an alteration in a cell resulting from the uptake of foreign nucleic acid, usually DNA. Use of the term “transfection” is not intended to limit introduction of the foreign nucleic acid to any particular method. Suitable methods include viral infection/transduction, conjugation, nanoparticle delivery, electroporation, particle gun technology, calcium phosphate precipitation, direct injection, and the like. The choice of method is generally dependent on the type of cell being transfected and the circumstances under which the transfection is taking place (i.e. in vitro, ex vivo, or in vivo). A general discussion of these methods can be found in Ausubel, et al, Short Protocols in Molecular Biology, 3rd ed., Wiley & Sons, 1995.

Methods of delivering the recombinant HBV cccDNA into an animal are known in the art. By “delivering” it is meant that any approach that is well known in the arts of plasmid delivery and transfection of the liver, but should not be considered to limit the scope of the present invention. For example, hydrodynamic injection (developed by Zhang et al. Hum Gene Ther 1999,10 (10): 1735-1737) as one of the known skills can be used for plasmid delivery.

As used herein, a “HBV marker” refers to any marker that can represent the HBV virus infection. The known HBV marker commonly used in the art includes, but not limited to “the DNA of HBV”, or the “protein of the HBV”, e.g., the HBsAg and HBeAg and so on. The method to determine the level of the HBV marker is known in the art, such as ELISA for the level of HBV protein, or the qRT-PCR analysis for the level of HBV DNA.

### DETAILED DESCRIPTION OF THE INVENTION

The present invention provides a recombinant HBV cccDNA comprising HBV genome or the fragment or variant thereof and site-hybrid insert, and a method to prepare said recombinant HBV cccDNA. Such recombinant HBV cccDNA comprises a site-hybrid insert after site-specific recombination and a HBV genome or the fragment or variant thereof. Particularly, the HBV genome or the fragment or variant thereof is flanked by the site-hybrid insert.

In one embodiment, the HBV genome is a full length genome of any genotype, or an over length genome of any genotype. In a preferable embodiment, the genotype of the genome is D. In more preferable embodiment, the full length of the genome of genotype D is specified in GeneBank JN664917.1, X02496, AY217370, or HPBHBVAA. In a further embodiment, the over length genome is 1.1 unit genome or 1.3 unit genome. In a particular embodiment, the HBV genome used in the present invention has or is consisted of the sequence represented by SEQ ID NO: 3 (GeneBank JN664917.1).

In one embodiment, the fragment of the HBV genome is a part of HBV genome. In a preferable embodiment, the fragment is a fragment of the HBV genome of any genotype, particularly the genotype D HBV genome (such as those specified in GeneBank JN664917.1, X02496, AY217370, or HPBHBVAA), more particularly a genotype D HBV genome represented by SEQ ID NO:3. Particularly, the HBV genomic fragment can replicate or express the one or more of the genes encoding envelope proteins, core/precore proteins, x protein and/or polymerase protein of HBV.

In one embodiment, the variant of the HBV genome can be the variant, that compared with the parent HBV genome or the fragment thereof, has at least one or several or more substitution(s), deletion(s) or insertion(s) in nucleotide sequence. For example, the variant of the HBV genome of the present invention can be the one that has one or several mutation on the gene encoding the HBV core protein which make the variant not able to replicate or express said protein, namely the variant of the HBV genome can be the HBV genome without the gene encoding the HBV core proteins (HBc). For example, the mutation can be on the start codon of the coding sequence of HBc. In one embodiment, the variant of the HBV genome can be represented by SEQ ID NO: 14.

In a further embodiment, the “site-hybrid insert” can be generated from any commercially available minicircle DNA producing parental vector that contains recombination substrates, such as attP and attB sites. In an embodiment, the recombination substrates are specific to recombinase, particularly integrase, e.g., wild-type phage integrases or mutants thereof, includes, but not limited to, the integrases of ΦC31, R4, TP901-1, ΦBT1, Bxb1, RV-1, AA118, U153, ΦFC1, and the like. Particularly, the “site-hybrid insert” is attR site. Most particularly, the attR site is represented by SEQ ID NO: 4. In a further embodiment, in the recombinant HBV cccDNA, the attR site is located immediately preceding the starting codon of preS1 gene, and between the terminal protein domain and spacer of the polymerase gene, particularly, the attR site is located between positions 2847 and 2848 of SEQ ID NO:3.

In a further embodiment, the method to prepare recombinant HBV cccDNA of the invention comprises
a) HBV genome or the fragment or variant thereof is inserted in and flanked by recombination substrate sites of minicircle DNA producing parental vector to form a parental HBVcircle construct;
b) The parental HBVcircle construct is transformed into the minicircle producer to generate recombinant HBV cccDNA via site-specific recombination.

In one embodiment, the minicircle producer can be microorganism which allow amplification of minicircle DNA producing parental vector, as well as generation of minicircle DNA upon the expression of recombinase. Particularly, the microorganism is bacterium, more particularly Escherichia sp, most particularly E. coli, e.g., strain ZYCY10P3S2T. In one embodiment, the minicircle producer is the microorganism wherein the recombinase can be expressed endogenously. Alternatively, the minicircle producer can be the microorganism to which the recombinase or the gene encoding said recombinase has been introduced and expressed therein.

In one embodiment, the minicircle DNA producing parental vector of the present invention can be any one known in the art, such as commercially available vectors from System Biosciences Inc. Particularly, the minicircle DNA producing parental vector used herein comprise recombination substrates, e.g., the recombination substrates specific to recombinase, particularly integrase, e.g., wild-type phage integrases or mutants thereof, includes, but not limited to, the integrase of ΦC31, R4, TP901-1, BT1, Bxb1, RV-1, AA118, U153, ΦFC1, and the like. More particularly, the minicircle DNA producing parental vector used herein is pMC.CMV-MCS-SV40polyA vector, which can be purchased from System Biosciences (catalogue number MN501A1).

In a further embodiment, in the parental HBVcircle construct, the HBV genome or the fragment or variant thereof is located between the recombination substrate sites. After site-specific recombination in the minicircle producer (e.g., E. coli), the HBV genome or the fragment or variant thereof in the obtained recombinant HBV cccDNA still maintain its ability to replicate or express. Particularly, the recombination substrate site is recombinase or integrase binding site particularly selected from attP or attB, more particularly, the attP used herein is represented by SEQ ID NO: 5, and/or the attB used herein is represented by SEQ ID NO: 6.

In the most preferable embodiment, the abovementioned HBV genome or the fragment or variant thereof is inserted in and flanked by attP and attB sites of pMC.CMV-MCS-SV40polyA vector, to replace the CMV-MCS-SV40polyA fragment already existed in the plasmid. Thus constructed recombinant plasmid is designated as parental HBVcircle, which DNA sequence is listed as SEQ ID NO: 1.

To generated recombinant HBV cccDNA using the abovementioned parental HBVcircle, the recombinant parental construct is then transformed into the minicircle producer E. coli strain ZYCY10P3S2T (commercially available from System bioscience Inc, catalogue number MN900A-1). Upon the expression of ΦC31 integrase and I-SceI homing endonuclease by adding arabinose, ΦC31 integrase catalyzes recombination between attB and attP sites, leading to the generation of

HBVcircle carrying a small attR site, as well as plasmid backbone circle. I-SceI homing endonuclease initiates the destruction of the parental unrecombinated DNA as well as the plasmid backbone circle by digesting the I-SceI recognition sites. Recombinant HBV cccDNA is then extracted from the minicircle producer E. coli. Thus generated recombinant HBV cccDNA is designated as HBVcircle, which DNA sequence is listed as SEQ ID NO: 2.

In one embodiment, the present invention relates to a method for expressing HBV antigen in vitro or a method for establishing an in vitro cccDNA based HBV model, including delivering the recombinant HBV cccDNA of the present invention into a cell line(e.g., cell line from hepatic cells, particularly those from hepatocyte, more particularly HepG2 or HepaRG) or primary cell(e.g., primary hepatic cell, particularly primary hepatocyte).

To express HBV antigens in vitro using the abovementioned prepared recombinant HBV cccDNA, the recombinant HBV cccDNA can be delivered into cultured cells using any known skills in the art, and consequently the cultured cells is introduced (e.g., transfected) by said recombinant HBV cccDNA. Therefore, HBV cccDNA could be conveniently introduced into all primary cells and cell lines by a simple transfection process, bypassing the restriction steps such as entry and cccDNA formation. The established cell culture models could be used for cccDNA research and anti-HBV drug evaluation, for example ETV, HAP 12, Pegasys or R848.

In one embodiment, the present invention also relates to a method for expressing HBV antigen and/or DNA in vivo or a method for establishing a cccDNA based HBV animal model., including delivering the recombinant HBV cccDNA of the present invention into an animal.

In one embodiment, the method of establishing a cccDNA based HBV animal comprises the step of delivering said recombinant HBV cccDNA into an animal and transfecting the hepatocytes of the animal.

To persistently express HBV antigens in vivo using the abovementioned recombinant HBV cccDNA, the recombinant HBV cccDNA can be delivered into animal (e.g., mouse) using any known skills in the art, and consequently the hepatocytes of the injected animal (e.g., mouse) is transfected by said recombinant HBV cccDNA.

In one embodiment, the animal can be a mammal or avian, e.g., mouse, particularly the mouse is C3H/HeN mouse. More particularly, the mouse used in the present invention is immunocompetent with functional innate and adaptive immunity.

In terms of delivery method of the recombinant HBV cccDNA, any approach that is well known in the arts of plasmid delivery and transfection of the liver cells can be applied in the present invention, but should not be considered to limit the scope of the present invention. In the present invention, for example, known skills such as hydrodynamic injection can be one of the methods for plasmid delivery. More specifically, in the example of the present invention, transfection of mouse liver cells with the recombinant plasmid is accomplished by hydrodynamic injection of the recombinant plasmid into the tail vein of mice. In order to allow the recombinant plasmid easily injected into tail vein of mice, the plasmid abovementioned is prepared in a biocompatible and non-immunogenic solution, such as phosphate buffer solution, but it should not be considered to limit the scope of the present invention.

In a further embodiment, once the recombinant HBV cccDNA of the present invention is delivered into a mouse and transfected the hepatocytes of the injected mouse, it behaves the same as natural HBV cccDNA and it can exist in an episomal form in the hepatocytes of the mouse for at least 30 days in the hepatocytes, particularly at least 37 days, 44 days or 51 days, which can be used as a HBV transcription template for production of viral antigens, replication intermediates, and mature virions which are released in bloodstream of the injected mouse. In a further embodiment, the expression HBV antigen persists for at least 30 days in the hepatocytes, particularly at least 37 days, 44 days or 51 days in the hepatocytes. Because the characteristics of this recombinant form HBV cccDNA and the cccDNA of natural infected HBV are very similar, therefore, the recombinant HBV cccDNA of the present invention can be used for evaluation and elucidation of mechanism of (chronic) hepatitis and anti-viral drug discovery research. Particularly, the animal model of the present invention is useful in the evaluation of a medicament for the treatment of hepatitis B virus infection, particularly, the ETV, HAP 2 and R848.

In a further embodiment, the animal (e.g., mouse) model of the present invention is based on the immunocompetent animal with functional innate and adaptive immunity, thus the induced liver histological and serological status is similar to that of healthy HBV carrier. Consequently the animal model of the present invention is an ideal model for mechanistic chronic hepatitis studies of hepatitis mechanism and drug evaluation.

Moreover, the present invention also relates to the kit or composition comprising the recombinant HBV cccDNA of the present invention. In a further embodiment, the composition or the kit comprising the recombinant HBV ccc DNA of the present invention can further contain biocompatible and non-immunogenic solution, such as phosphate buffer solution.

Furthermore, the present invention also relates to a method for in vitro anti-HBV drug evaluation or for in vitro evaluating a medicament for the treatment of hepatitis B virus infection in cell culture medium, including that

(1) delivering the recombinant HBV cccDNA of the present invention into a cell (e.g., a cell line (e.g., cell line from hepatic cells, particularly those from hepatocyte, more particularly HepG2 or HepaRG) or primary cell (e.g., primary hepatic cell, particularly primary hepatocyte)),

(2) treating the cell with the drug or medicament to be evaluated for 1-30 days, particularly 2-10 days,

(3) detecting the level of the HBV marker in the cell, and

(4) the reduced the level of the HBV marker in the cell treated by said drug or medicament indicating the drug being an effective anti-HBV drug or the medicament being effective in the treatment of hepatitis B virus infection.

Furthermore, the present invention also relates to a method for in vivo anti-HBV drug evaluation or for in vivo evaluating a medicament for the treatment of hepatitis B virus infection, including that

(1) delivering the recombinant HBV cccDNA of the present invention into an animal (e.g., mouse, particularly C3H/HeN mouse),

(2) administering the drug or medicament to be evaluated into the animal,

(3) detecting the level of the HBV marker in the blood (e.g., serum) of the animal , and

(4) the reduced the level of the HBV marker in the blood of the animal receiving said drug or medicament indicating the drug being an effective anti-HBV drug or the medicament being effective in the treatment of hepatitis B virus infection.

In one embodiment, the hepatitis B virus infection is chronic hepatitis B virus infection.

### EXAMPLES

The following examples are put forth so as to provide those of ordinary skill in the art with a complete disclosure and description of how to make and use the present invention. The examples, which are intended to be purely exemplary of the invention and should therefore not be considered to limit the invention in any way, also describe and detail aspects and embodiments of the invention discussed above. The examples are not intended to represent that the experiments below are all or the only experiments performed.

### Materials and Methods

### Recombinant DNA Techniques

Standard methods were used to manipulate DNA as described in Sambrook, J. et al., Molecular cloning: A laboratory manual; Cold Spring Harbor Laboratory Press, Cold Spring Harbor, N.Y., 1989. The molecular biological reagents were used according to the manufacturer's instructions.

### Gene Synthesis

Desired gene segments were prepared from oligonucleotides made by chemical synthesis. The 100-600 bp long gene segments, which were flanked by singular restriction endonuclease cleavage sites, were assembled by annealing and ligation of oligonucleotides including PCR amplification and subsequently cloned into the pCR2.1 -TOPO-TA cloning vector (from Invitrogen Corp., USA) via A-overhangs. The DNA sequence of the subcloned gene fragments were confirmed by DNA sequencing.

### Cell Lines

The human hepatoma derived cell line HepG2 (Purchased from ATCC, ATCC® HB-8065) were cultured in DMEM/F12 (from Invitrogen) supplemented with 10% fetal bovine serum (from Invitrogen), 2 mM L-glutamine, 100 U/ml penicillin, and 100 μg/ml streptomycin at 37° C. under humidified air containing 5% CO₂. The proliferating HepaRG cells were purchased from Biopredic International (Rennes, France). HepaRG cells were amplified and differentiated following manufacture's protocol.

### Animal Study

All procedures in this study were in compliance with local animal welfare legislation and applicable guidelines.

C3H/HeN mice (male, aged 4-6 weeks) were obtained from Vital River Laboratories Co. Ltd, Beijing, China. CBA/J mice (male, aged 4˜6 weeks) were obtained from HFK Bioscience Co., Ltd., Beijing, China. Mice were housed in polycarbonate cages with corncob bedding under controlled temperature (21-25° C.), humidity (40-70%), and a 12-hour light/12-hour dark cycle (7:00 AM to 7:00 PM lights on). Mice were provided ad libitum access to normal diet (Rodent Diet #5001, PMI Nutrition International, LLC, IN, USA) and sterile water.

The animals were grouped based on Day-1 body weights. On Day 0, all animals were subjected to hydrodynamic injection through tail vein within 5 seconds with 2.5-20 μg DNA in a volume (mL) of saline equivalent to 8% of body weight (g) (Liu, et al., 1999, Gene Ther, 6: 1258-66, Zhang, et al., 1999, Hum Gene Ther, 10: 1735-7). After animal exclusion due to technical failure of hydrodynamic injection or low HBV marker expression on Day 1 or Day 3, the remaining mice were maintained for long term evaluation. Blood samples were collected for serum preparation on indicated time post HDI injection.

For compound treatment, on Day 20 post HDI (DAY-3 of compound treatment), C3H/HeN mice were divided into 4 groups based on serum HBsAg levels and body weights on DAY 20. ETV and R848 were diluted in saline from stock solutions on treatment days. Vehicle was RC591. All test compounds were given orally with indicated dose and frequency.

### Transient Transfection

X-TREMEGENE HP DNA transfection reagent (from Roche) was used for transfection. One day before transfection, cells were trypsinized and seeded onto plates, Cells were seeded at 0.8×10⁵/well in 24 well plates for HepG2 and proliferation HepaRG cell line, and 3×10⁵/well in 24 well plates for differentiated HepaRG cell line.

### Detection of HBV Antigen

HBeAg or HBsAg were measured by using the HBeAg or HBsAg ELISA kit (from Autobio) according to the manufacturer's direction.

### Detection of HBV DNA

HBV DNA in cell culture supernatant or mouse serum was extracted using MagNA Pure 96 System MagNA Pure 96 System (from Roche). HBV DNA levels were determined via RT-PCR. The primer and probe sequences are shown below.

Forward primer:
5′-GCTGGATGTGTCTGCGGC-3′ (372-389);
Reverse primer:
5′-GAGGACAAACGGGCAACATAC-3′ (459-479);
Probe:
5′-CATCCTGCTGCTATGCCTCATCTTCTTG-BHQ-2-3′
(409-436).

Plasmid pBR322-HBV1.3 (SEQ ID NO: 7) with appropriate dilution was used as standard for RT-PCR.

### Cell Viability Assay

Cell viability was determined by the amount of albumin secreted into the supernatant using the Albumin AlphaLISA kit (from PerkinElimer).

### DNase Digestion

The cell lysate were digested by the DNase I kit (from Sigma) following manufacturer's directions.

### Hirt DNA Extraction

Hirt DNA was prepared following previously described procedures, with slightly modifications (Cai, et al., 2013, Methods Mol Biol, 1030: 151-61). Briefly, HepG2 cells (1×10⁶) or homogenized liver tissues (50 mg) were suspended in 500 μl 50 mM Tris-HCl buffer (pH7.4) with 10 mM EDTA. Then 1241 10% SDS was added and 100 μ1 2.5M KCl was added and mixed gently. After a centrifugation at 4° C. for 10 min, the supernatant was extracted with phenol and phenol:chloroform:isoamyl alcohol (25:24:1) and phenol respectively. Precipitate the DNA with ethanol, and the nucleic acids were dissolved in TE buffer (10 mM Tris-HCl, 1 mM EDTA, pH 8.0).

cccDNA Realtime PCR

HBV cccDNA specific primer and probe set was used to detect cccDNA:

cccDNA-F,
5′-CTCCCCGTCTGTGCCTTCT-3′ (1545-1563);
cccDNA-R:
5′-GCCCCAAAGCCACCCAAG-3′ (1883-1900);
cccDNA-probe:
5′-TARMA + CGTCGCATGGARACCACCGTGAACGCC +
BHQ-2-3′ (1602-1628).

### Detection of HBV Nucleocapsid

The transfected HepG2 cells were lysed using lysis buffer (50 mM Tris-HCl, pH8.0, 100 mM NaCl, 1% CA-630, 1×EDTA free proteinase inhibitor). After incubation at 4° C. for 1 h with agitation, cytoplasmic lysate was cleared by centrifugation. The lysate was separated by electrophoresis in 1.5% agarose gel and then transferred onto PVDF membrane (for Western blotting) or a positively charged nylon membrane (for southern blotting), for detecting the capsid protein and encapsidated DNA respectively.

### Western Blot

After SDS-PAGE, gels were transferred to PVDF membrane by using iblot system (from Invitrogen). After blocking, membranes were incubated with primary antibody, rabbit anti-HBV core antigen (from Dako) and mouse monoclonal anti-actin (from Sigma). After several washes, membranes were then incubated with appropriate secondary antibodies (from KangChen) coupled to horse radish peroxidase (HRP). After washes, signals were visualized using Western Pico Super ECL reagent (from Pierce).

### Southern Hybridization

The sample was loaded to an electrophoresis of 1.5% agarose gel in 1×TAE buffer for 2-3 hours. After denaturation and neutralization, DNA was blotted onto a Hybond-N+ membrane (from GE Healthcare) in 20×SSC and hybridized with a DIG-labeled HBV DNA probe. After incubating blots with an alkaline-phosphatase-conjugated anti-DIG antibody, hybridization signals were detected in a standard chemiluminescence reaction.

### Immunofluorescence

Cells were cultured on chamber slides (from Permanox), fixed with 4% paraformaldehyde in PBS and permeabilized with permeabilizing buffer (5% BSA+0.5% triton in PBS). The cells were stained with rabbit anti-HBV core antigen (from Dako) and mouse monoclonal anti-HBV surface antigen (from Invitrogen). Antibodies were diluted in PBS containing 5% FBS. After washing with PBS, bound antibodies were labeled with secondary antibodies, Alexa Fluor 594 nm goat anti-rat and Alexa Fluor 488 nm donkey anti-rabbit (from Invitrogen). Following several additional washes, cells were stained with DAPI (from Invitrogen) and observed under a Nikon inverted IF microscope.

### Chromatin Immunoprecipitation

ChIP assay was performed with an EpiTect Chip One-Day Kit (from Qiagen) by following the procedures provided by the manufacturer with slight modifications. The cells were fixed in 1% formaldehyde at 37° C. for 10 minutes. After stop the fix, the cells were pelleted at 800 g for 10 minutes at 4 ° C. and resuspended by addition of immunoprecipitation lysis buffer supplemented with proteinase inhibitor cocktail. Five hundred microliters of the cell lysates were sonicated by cup horn (Sonicator XL2020, Misonix) at a setting of 26 W for 2 seconds on and 15 seconds off, 16 seconds (8 times per round) for the total time, 9 rounds. This sonication condition has been showed steadily breaking cellular DNA into 500-800 bp fragments. For pre-clear, immunoprecipitation and DNA extraction, we strictly followed the instruction provided in the EpiTect ChIP One-Day Kit (from Qiagen).The obtained DNA was subjected to quantitative analysis by real time PCR with the specific cccDNA primers:

Forward,
5′-CTGAATCCTGCGGACGACCC-3′ (1441-1460 nt);
Reverse,
5′-CCCAAGGCACAGCTTGGAGG-3′ (1889-1869 nt).

### Immunohistochemical Staining

Resected liver tissue samples were immediately immersed in 4% formalin and fixed for 18 to 24 hours and paraffin-embedded. Immunohistochemical staining was carried out on tissue sections by using anti-HBc multiclonal antibody (from Dako) to detect the core antigen expression. The Immunoreactive score (IRS) semi-quantitative scoring system are used for evaluating the proportion of HBc-positive cells and the intensity of staining. Staining intensity was graded as 0 (negative), 1 (weak), 2 (moderate), and 3 (strong); percentage of positive cells was scored as 0 (negative), 1 (<25%), 2(25%˜50%), 3 (50%˜75%), 4 (>75%). The two scores were multiplied and the IRS was determined.

### Example 1

### Design and Production of HBVcircle

Plasmid pBR322-HBV1.3 containing a 1.3 unit over length genotype D HBV genome (GeneBank JN664917.1) and the sequence of said plasmid is listed as SEQ ID NO: 7. The parental minicircle DNA vector plasmid, pMC.CMV-MCS-SV40polyA was purchased from System Biosciences (Catalogue Number MN501A1, sequence listed as SEQ ID NO: 13).

For the parental HBVcircle-CMV-HBV1.1 construct, a 1.1 unit over length HBV genome starting from nucleotide 1805 to 3182 and 1 to 1990 of the genotype D HBV genome was retrieved from pBR322-HBV1.3 via PCR and then cloned into pMC.CMV-MCS-SV40polyA vector using SalI and NheI sites. The sequence for this parental HBVcircle-CMV-HBV1.1 construct is listed as SEQ ID NO: 8.

For the parental HBVcircle-HBV1.3 construct, the pMC.CMV-MCS-SV40polyA vector was digested with SmaI and KpnI (purchased from New England Biolabs Ltd). To generate the 1.3 unit over length HBV genome insert (listed as SEQ ID NO: 9), SmaI site containing forward primer

5′-TGGGCTCCCCGGGCGCGCAATCTAAGCAGGCTTTCACT-3′,

and KpnI site containing reverse primer

5′-ATGTGGTACCACATCATGATGCTGATTACCCCCAACTGAGAGAACTC
AAAGGTTACCCCAGTTGGGGGATCTCGTACTGAAGGAAAGA-3′

were used to generate a 4.2 kb fragment (listed as SEQ ID NO: 10) via PCR using pBR322-HBV1.3 as template. The PCR fragment was restricted with SmaI and KpnI, and ligated with the pMC.CMV-MCS-SV40polyA vector that had been digested by the same enzymes to yield the parental plasmid. The sequence for the parental HBVcircle-HBV1.3 construct is listed as SEQ ID NO: 11.

For the parental HBVcircle construct, the pMC.CMV-MCS-SV40polyA vector was digested with SmaI and KpnI. The full HBV genome insert starting from nucleotide 2848 to 3182 and 1 to 2847 of the genotype D HBV genome flanked by attB and attP sites, as well as SmaI and KpnI sites was directly gene synthesized (sequence listed as SEQ ID NO: 12), digested and ligated with the pMC.CMV-MCS-SV40polyA vector that had been digested by the same enzymes to yield the parental plasmid. The sequence for the parental HBVcircle construct is listed as SEQ ID NO: 1.

Minicircle DNA was produced using MC-Easy Minicircle DNA Production Kit following manufacturer's instructions (System Biosciences, MN925A-1). HBVcircle, HBVcircle-CMV-HBV1.1 and HBVcircle-HBV1.3 DNA was generated respectively from their corresponding parental plasmid as listed above in minicircle producer E.coli strain ZYCY10P3S2T upon switching on ΦC31 integrase and I-SceI genes expression (FIG. 1A). For the HBVcircle DNA, the 39 nucleotides attR site insertion (SEQ ID NO: 4) in HBVcircle is located between 2847 and 2848 positions of SEQ ID NO: 3 immediately preceding the starting codon of preS1 gene, and between the TP (terminal protein) domain and spacer of the polymerase gene (FIG. 1B). The entire sequence of HBVcircle is listed as SEQ ID NO: 2. The size and sequence of HBVcircle DNA were verified by agarose gel electrophoresis and Sanger sequencing, respectively (FIG. 1C).

### Example 2

### Assessing HBV Replication After HBVcircle Transfection In Vitro

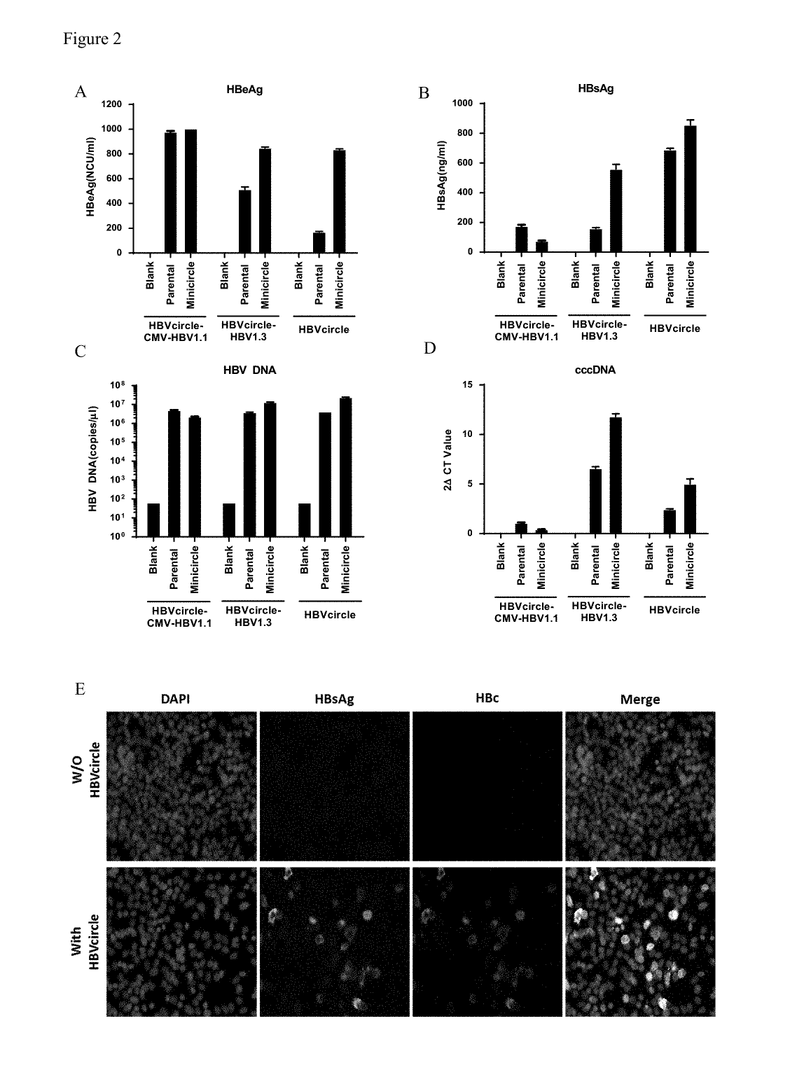

HBVcircle, HBVcircle-CMV-HBV1.1 and HBVcircle-HBV1.3 as well as their parental plasmids were transiently transfected into HepG2 cells for viral replication testing. 72 hours after transfection, cell culture supernatants were collected and subjected to ELISA and qRT-PCR analysis. HBeAg, HBsAg and HBV DNA were highly abundant in supernatants, suggesting robust viral replication (FIG. 2A, B and C). Cells were lysed and total DNA was extracted, cccDNA was quantified using realtime PCR with a cccDNA specific primer and probe set (FIG. 2D). Compared with parental HBVcircle-HBV1.3 plasmid, which carried traditional 1.3 units HBV genome over length design, HBVcircle showed at least comparable or higher HBV markers expression.

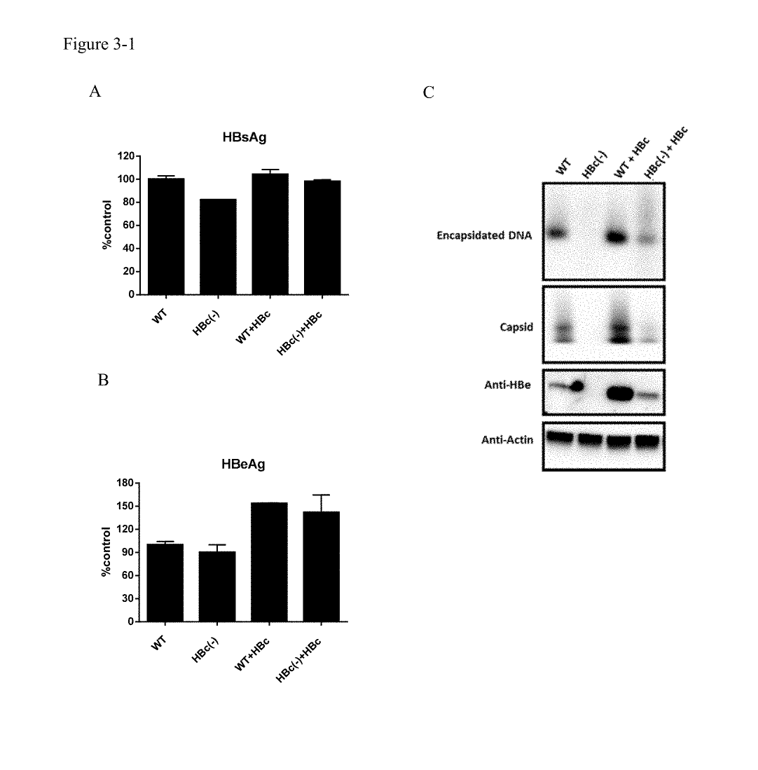

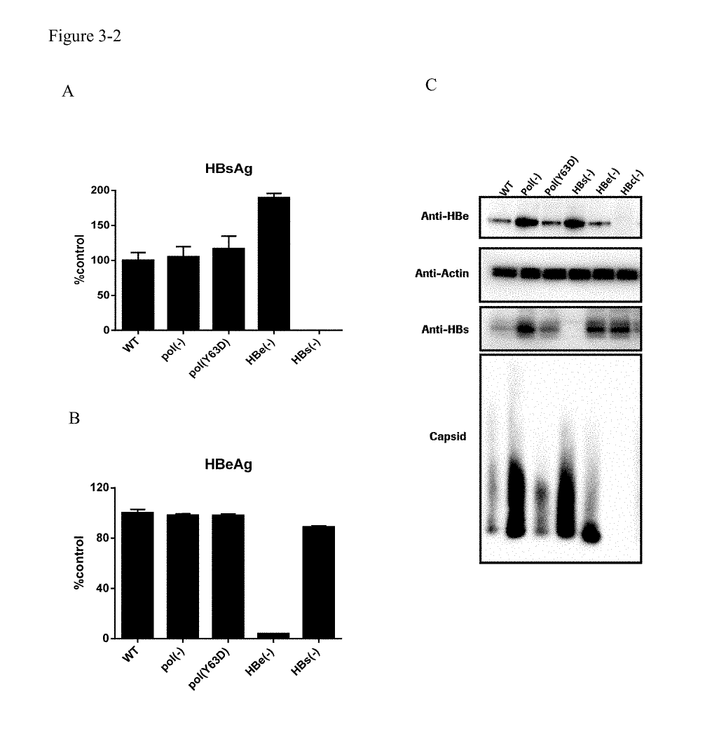

In addition, HBsAg and HBV core (HBc) proteins were readily detectable in HBVcircle transfected cells with immunofluorecent staining (FIG. 2E). In order to determine the impact of HBc deficiency on HBV replication, an HBc(−) HBVcircle was constructed as SEQ ID NO: 15, in which the start codon of HBc was mutated. When these two constructs were transfected into HepG2 cells, HBsAg and HBeAg expression were similarly expressed (FIG. 3-1A and 3-1B). Intracellular HBV capsid and encapsidated HBV DNA were only detected in wild type, but not HBc(−) HBVcircle transfected cells. When HBc was supplemented in trans, the defects were successfully rescued (FIG. 3-1C). Additional HBV mutants were also generated and HBV replication markers were tested as indicated in FIG. 3-2A, 3-2B and 3-2C. These mutants including HBVcircle Pol(−), in which the start codon of the HBV polymerase gene was mutated (SEQ ID NO: 16), rendering the virus defective in polymerase expression and unable to package viral RNA (Nguyen et al., J Virol. 2008;82:6852-6861); HBVcircle Pol(Y63D), in which HBV polymerase carried a Y63D mutation (SEQ ID NO: 17), rendering the virus defective in DNA synthesis but fully functional in RNA packaging (Lanford et al., J Virol. 1997;71:2996-3004); HBVcircle HBs(−), in which two premature stop codons were introduced into the preS2 and S coding regions (SEQ ID NO: 18); HBVcircle HBe(−), in which a premature stop codon mutation, G1896A, was introduced into the precore gene (SEQ ID NO: 19).

These data clearly demonstrated that HBVcircle was fully competent for supporting high level HBV replication once introduced into hepatic cells.

### Example 3

### Assessing cccDNA Markers In Vitro

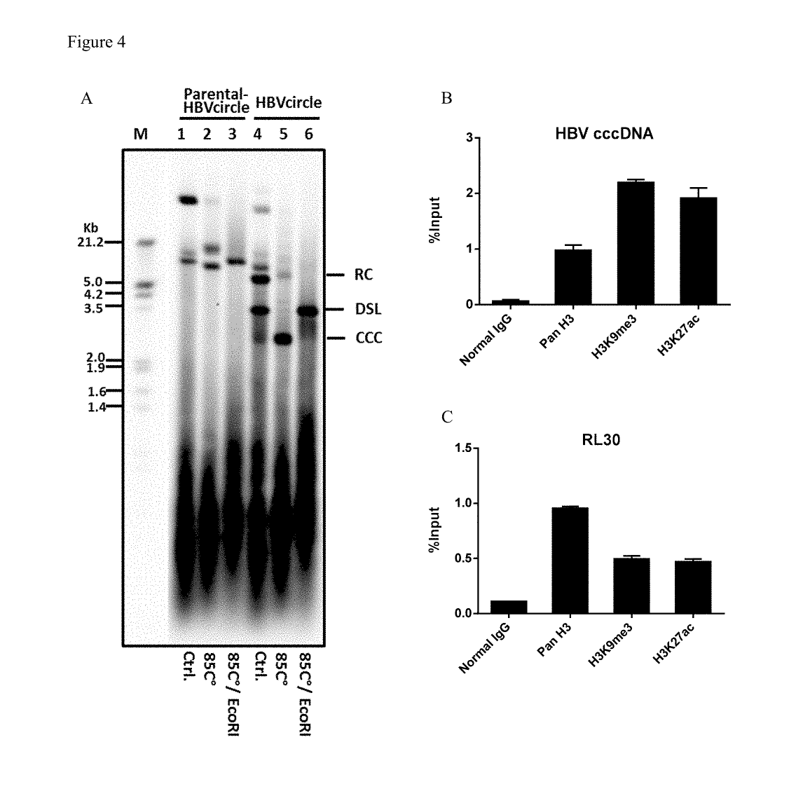

The presence of cccDNA in nucleus is one unique characteristic of HBV. In order to determine whether HBVcircle is capable of forming cccDNA in the nucleus of hepatic cells, southern blot and CHIP analysis were performed. For southern blot analysis, parental-HBVcircle or HBVcircle was firstly transfected into HepG2 cells and Hirt DNA was then prepared (Cai, et al., 2013, Methods Mol Biol, 1030: 151-61). The supercoiled heat resistant cccDNA bands appeared on southern blot only in HBVcircle transfected cells, but not in parental-HBVcircle transfected cells. Upon EcoRI linearization, cccDNA band disappeared (FIG. 4A, RC: relaxed circle; DSL: double strand linear; CCC: cccDNA). CHIP analysis was also conducted using HBVcircle transfected cells. Consistent with previous publications, epigenetic modifications including trimethylated lysine 9 (H3K9me3) and acetylated lysine 27 (H3K27ac) were associated with cccDNA (Liu, et al., 2013, PLoS Pathog, 9: e1003613). In the meanwhile, similar levels of total H3 (Pan H3) was observed between HBV cccDNA and host RL30 gene (FIG. 4B and C, respectively). Collectively, these data demonstrated the existence of authentic cccDNA as minichromosomes in HBVcircle transfected cells, which further supported that HBVcircle could be used as a surrogate for studying the natural HBV cccDNA.

### Example 4

### In Vitro Anti-HBV Drug Evaluation Using HBVcircle

Next, the feasibility of evaluating anti-HBV drugs using HBVcircle system in cell culture models was assessed. HepG2 cells or proliferating HepaRG cells were transiently transfected with HBVcircle and then treated with indicated concentrations of ETV, HAP 12 (an HBV capsid assembly inhibitor, which belongs to heteroaryldihydropyrimidine (HAP) chemical series, and was published as Example 12 in Bourne et al., J Virol. October 2008; 82(20): 10262-10270) or Pegasys for 6 days. Supernatants were collected and HBsAg, HBeAg and albumin ELISA were performed. Cells were lysed and cell lysates were subjected to southern blot analysis for encapsidated HBV DNA detection, as well as western blot analysis for HBV capsid, HBc and beta-actin detection with specific antibodies respectively.

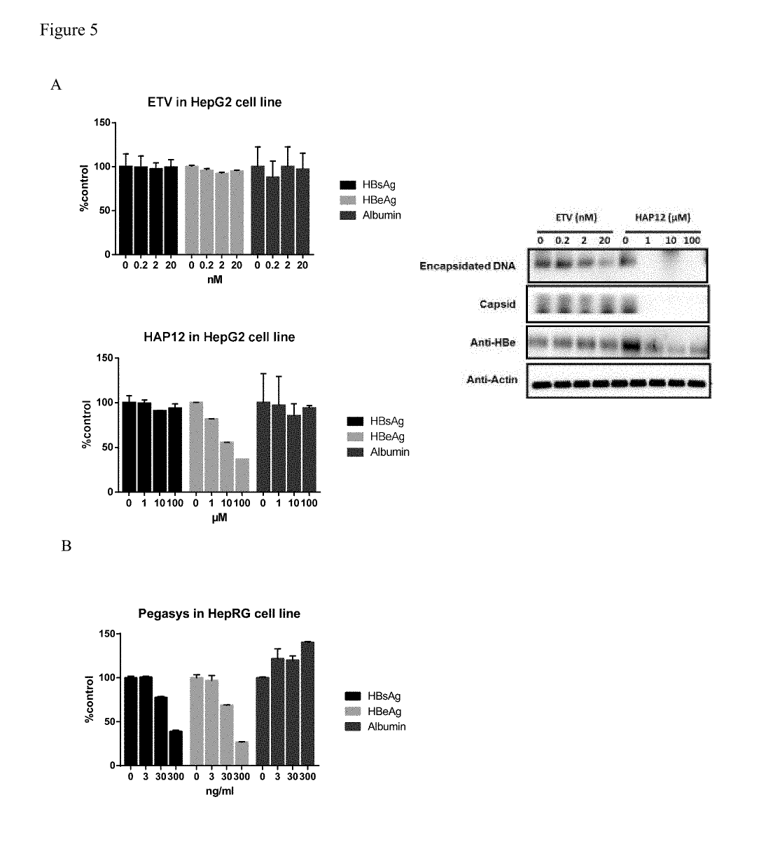

In HBVcircle transfected HepG2 cells, Entecavir (ETV), an approved nucleoside analogue for treating CHB, efficiently blocked HBV DNA replication in a dose dependent manner, while did not affect other viral proteins expression (FIG. 5A, upper left panel and right panel). On the other hand, HAP 12 blocked capsid formation, leading to abrogation of HBV DNA replication (Bourne, et al., 2008, J Virol, 82: 10262-70). We also observed that HAP 12 specifically reduced HBeAg secretion, but did not affect HBsAg or albumin, in a dose dependent manner (FIG. 4A, lower left panel and right panel).

Pegasys (pegylated interferon alpha-2a) is another approved drug for treating CHB and could activate multiple host mechanisms to suppress HBV replication. When treating HBVcircle transfected HepaRG cells with Pegasys, both HBsAg and HBeAg production were inhibited dose dependently (FIG. 5B). These results suggest that HBVcircle could be used for evaluating different classes of anti-HBV drugs in vitro in cell culture models.

### Example 5

### Establishment of Persistent HDI Mouse Model with HBVcircle

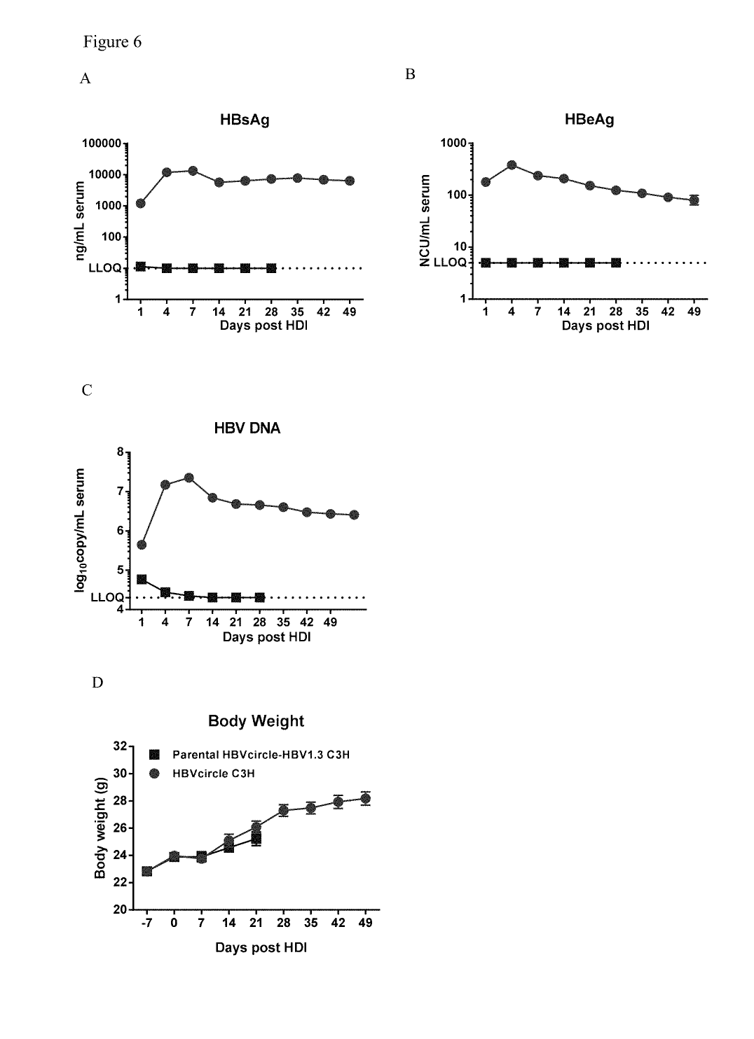

To test HBV replication and persistent time in vivo, 10μg of HBVcircle, HBVcircle-HBV1.3, along with the parental plasmid of HBVcircle-HBV1.3 were hydrodynamically injected into the tail vain of C3H/HeN mice (male, aged 4-6 weeks). At indicated time points post HDI, blood samples were collected for HBV markers testing, including HBsAg, HBeAg and HBV DNA (FIG. 6). Animal numbers of FIG. 6 at indicated time points were shown in Table 1. C3H/HeN mice injected with HBVcircle demonstrated extremely high level and stable HBV markers expression compared to the other group. All HBV markers persisted beyond 7 weeks after injection. In contrast, the parental HBVcircle-HBV1.3 construct, which has the classical HBV1.3 design as in the pBR322-HBV1.3 construct, failed to support HBV persistence, as the HBsAg rapidly decreased and became undetectable beyond day 14. In the meanwhile, mice body weight was monitored during the entire experiment, and there was no significant difference among all groups in each strain (FIG. 6D).

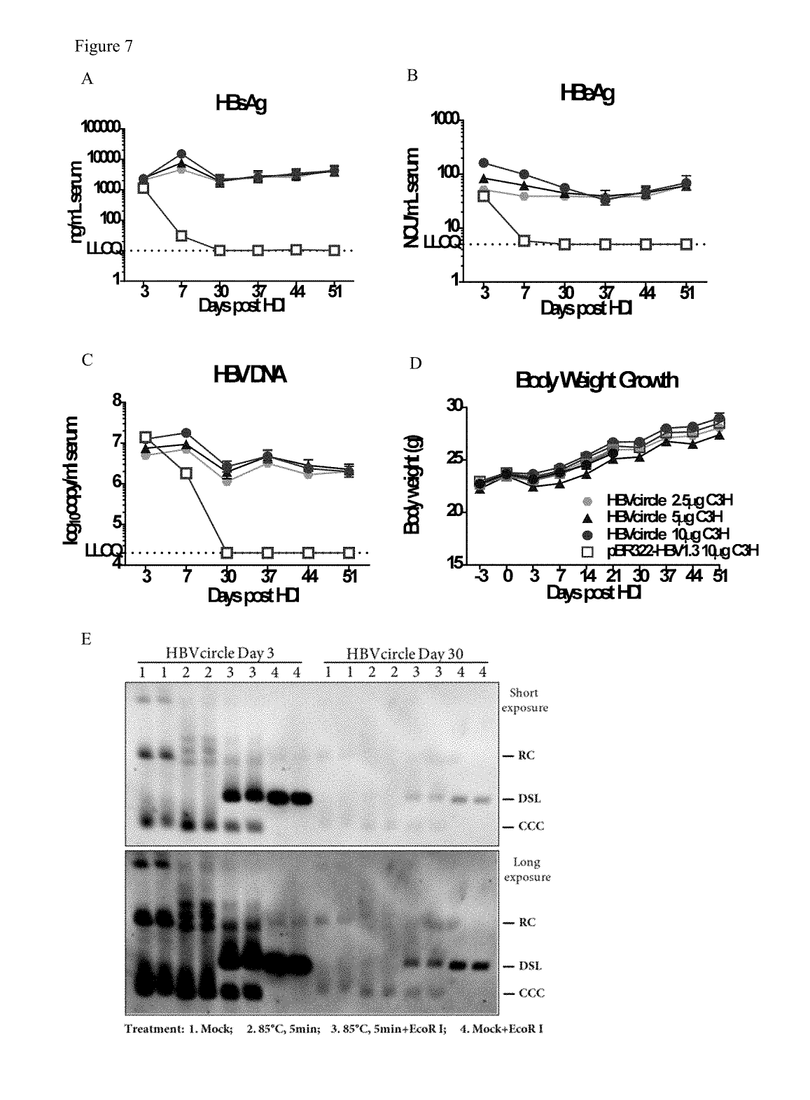

In order to understand the impact of DNA amount during HDI on HBV persistency, 4 different doses of HBVcircle, together with a control plasmid pBR322-HBV1.3, were injected into C3H/HeN mice. HBV markers in serum were monitored for 51 days. Animal numbers of FIG. 7 at indicated time points were shown in Table 2. As shown in FIG. 7, all mice in 2.5 μg, 5 μg and 10 μg groups maintained highly level of viral replication and persisted for at least 51 days, and despite an initial dose dependent pattern of viral markers expression was observed, there was no significant difference at later time points beyond 30 days. The pBR322-HBV1.3 group, which carried a different plasmid backbone and 1.3 over-length of HBV genome, did not persist well.

Next, in order to detect cccDNA in mouse liver, we sacrificed 2 mice each time from the HBVcircle 10 μg group on day 3 and day 30. Mouse livers were harvested and Hirt DNA was prepared for southern blot analysis. As shown in FIG. 7D, the heat resistant cccDNA was clearly detectable on day 3 after HDI. And cccDNA level was decreased, but still detectable on day 30. When linearized by EcoRI digestion, the fast migrating supercoiled cccDNA bands disappeared as expected. These results suggested that the persistent phenotype was driven by authentic cccDNA in mouse liver.

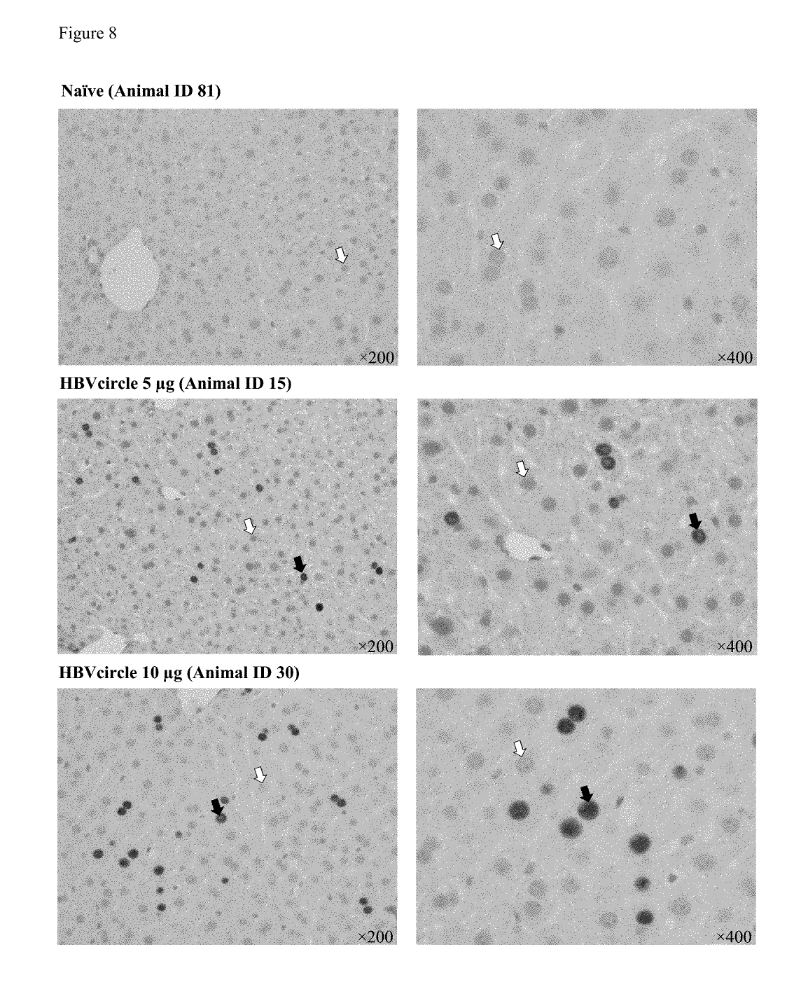

Immunohistochemistry (IHC) staining of HBc was also performed with selected mice as indicated in Table 3 and FIG. 8 at day 120 post HDI injection. The results demonstrated that HBV replication persisted at least 120 days in these mice, and HBc was predominantly presented in the nucleus of HBV replicating hepatocytes, a phenotype similar as observed in HBV chronically infected people in immune tolerant phase (Hsu, et al., 1987, J Hepatol.;5(1):45-50).

TABLE 1
Animal numbers of FIG. 6 at indicated time points

| | Days post HDI | | | | | | | | |
|---|---|---|---|---|---|---|---|---|---|
| Injected plasmid | 1 | 4 | 7 | 14 | 21 | 28 | 35 | 42 | 49 |
| Parental HBVcircle- | 16 | 7 | 7 | 6 | 5 | 5 | — | — | — |
| HBV1.3 C3H | | | | | | | | | |
| HBVcircle C3H | 16 | 16 | 16 | 11 | 11 | 11 | 11 | 11 | 11 |

TABLE 2
Animal numbers of FIG. 7 at indicated time points

| | Days post HDI | | | | | |
|---|---|---|---|---|---|---|
| Injected plasmid | 3 | 7 | 30 | 37 | 44 | 51 |
| HBVcircle 2.5 μg | 11 | 9 | 9 | 9 | 9 | 9 |
| HBVcircle 5 μg | 11 | 9 | 8 | 8 | 8 | 8 |
| HBVcircle 10 μg | 11 | 9 | 9 | 9 | 7 | 7 |
| pBR322-HBV1.3 10 μg | 11 | 9 | 9 | 9 | 9 | 9 |

TABLE 3
IHC results quantification for FIG. 8

| | | Injected plasmid | | | | |
|---|---|---|---|---|---|---|
| | | Naive | HBVcircle 5 μg | | HBVcircle 10 μg | |
| | | Animal ID | | | | |
| | | 81 | 15 | 85 | 29 | 30 |
| Serum HBV | HBsAg | ND⁽⁵⁾ | 10033 | 13008 | 13509 | 10026 |
| markers at Day | (IU/ml) | | | | | |
| 51⁽⁶⁾ | HBeAg | ND | 74 | 132 | 197 | 115 |
| | (NCU/ml) | | | | | |
| | DNA | ND | 6.71 | 6.84 | 6.82 | 6.53 |
| | (Log10 | | | | | |
| | copy/ml) | | | | | |
| Serum HBV | HBsAg | 10 | 1300 | 70 | 1590 | 1478 |
| markers at Day | (IU/ml) | | | | | |
| 120⁽⁶⁾ | HBeAg | 5 | 41 | 11 | 54 | 56 |
| | (NCU/ml) | | | | | |
| | DNA | 4.30 | 4.88 | 4.30 | 4.60 | 4.57 |
| | (Log10 | | | | | |
| | copy/ml) | | | | | |

| | | | | | |
|---|---|---|---|---|---|
| Positive staining percent⁽¹⁾ | 0/0/0/0/0 | 1/1/0/0/0 | 0/1/0/0/0 | 1/1/1/1/1 | 1/1/1/1/1 |
| Staining intensity⁽²⁾ | 0/0/0/0/0 | 2/2/0/0/0 | 0/1/0/0/0 | 1/1/1/1/1 | 1/3/2/2/2 |
| IRS⁽³⁾ | 0/0/0/0/0 | 2/2/0/0/0 | 0/1/0/0/0 | 1/1/1/1/1 | 1/3/2/2/2 |
| Accumulated IRS⁽⁴⁾ | 0 | 4 | 1 | 5 | 10 |

⁽¹⁾Positive staining percent scores: 0 (negative); 1 (positive cells <25%); 2 (positive cells 25%~50%); 3 (positive cells, 50%~70%); 4 (positive cells >75%).
⁽²⁾Staining intensity from weak to strong: 0~3.
⁽³⁾IRS: positive staining percent scores × staining intensity scores.
⁽⁴⁾Accumulated IRS: sum of five replicates IRS.
⁽⁵⁾ND: Not determined
⁽⁶⁾LLOQ for HBsAg is 10 IU/ml; LLOQ for HBeAg is 5 NCU/ml; LLOQ for HBV DNA is 4.30 Log 10(copy/ml).

### Example 6

### In Vivo Anti-HBV Drug Evaluation Using HBVcircle

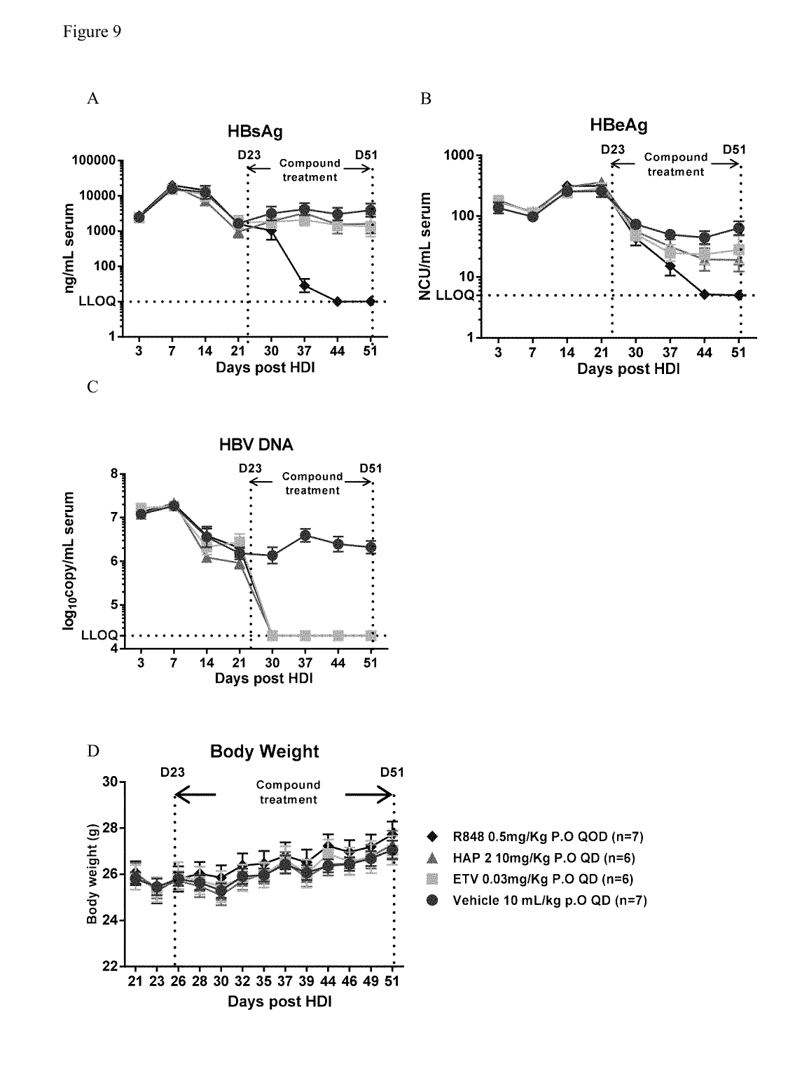

Establishment of persistent high level HBV replication with HBVcircle in immune competent mice may allow evaluation of anti-HBV drugs with different mechanism of actions (MoA). To test this, we first injected C3H/HeN mice with 10 μg HBVcircle and waited for 22 days before initiation of antiviral drug treatment. On day 23, the mice were divided into four groups with 6-7 mice/group and vehicle, ETV (0.03mg/kg, QD), HAP 2 (an HBV capsid assembly inhibitor, which belongs to heteroaryldihydropyrimidine (HAP) chemical series, and was published as Example 2 in patent WO2014/037480, 10 mg/kg, QD) and R848 (Resiquimod, a TLR7 agonist, the structure was published in Hemmi et al., Nature Immunology 3, 196-200 (2002), 0.5 mg/kg, QOD) were orally given to each group of mice for 29 days. As shown in FIG. 9, ETV, HAP 2 and R848 treatment efficiently reduced HBV DNA in serum to undetectable level. In addition, R848 also greatly reduced HBsAg and HBeAg, rendering all three HBV serum markers undetectable from day 44 (22 days on treatment). The results in this example clearly indicated that the model established by the recombinant HBV cccDNA of the present invention was an effective method used for the drug evaluation.

### Example 7

### Establishment of Persistent HDI Mouse Model with Additional Mouse Strain and HBV Genotypes

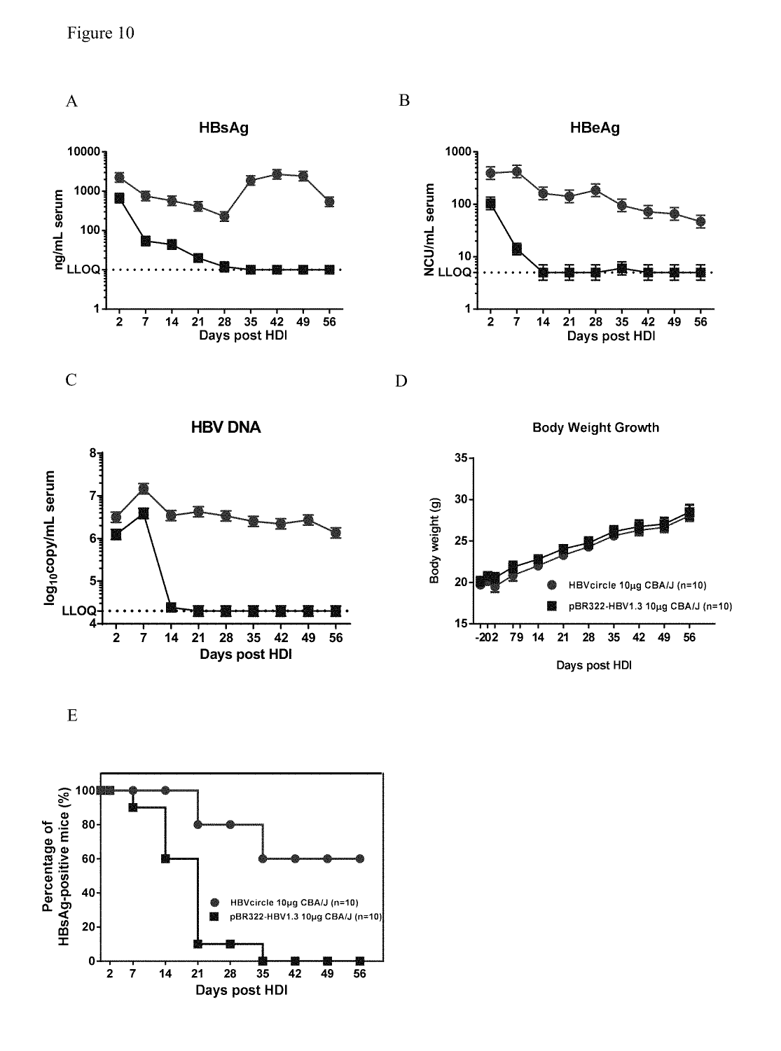

In addition to C3H/HeN mice, another immune competent mouse strain, CBA/J, was also evaluated for their ability to support persistent HBV replication. In the experiment shown in FIG. 10, HBVcircle or pBR322-HBV1.3 was hydrodynamically injected into the tail vain of CBA/J mice (male, aged 4-6 weeks). At indicated time points post HDI, blood samples were collected for HBV markers testing, including HBsAg, HBeAg and HBV DNA. HBV replication persisted for at least 56 days in 60% of HDI injected mice.

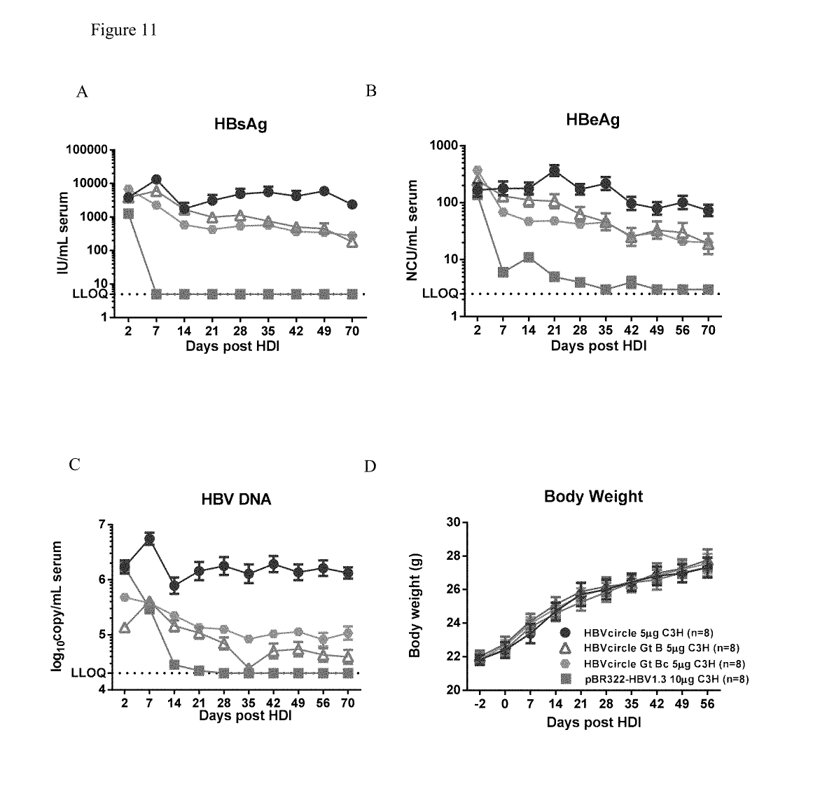

In addition to genotype D HBV sequence, two HBVcircle constructs with genotypes B HBV sequence were also evaluated. As shown in FIG. 11, both HBVcircle Gt B (SEQ ID No:22, derived from GeneBank AY220698), as well as HBVcircle Gt Bc (SEQ ID No:23, derived from GeneBank GQ205440), showed comparable persistency as the original HBVcircle in C3H/HeN mice.

### Example 8

### In Vivo Characterization of HBV Mutants Using HBVcircle

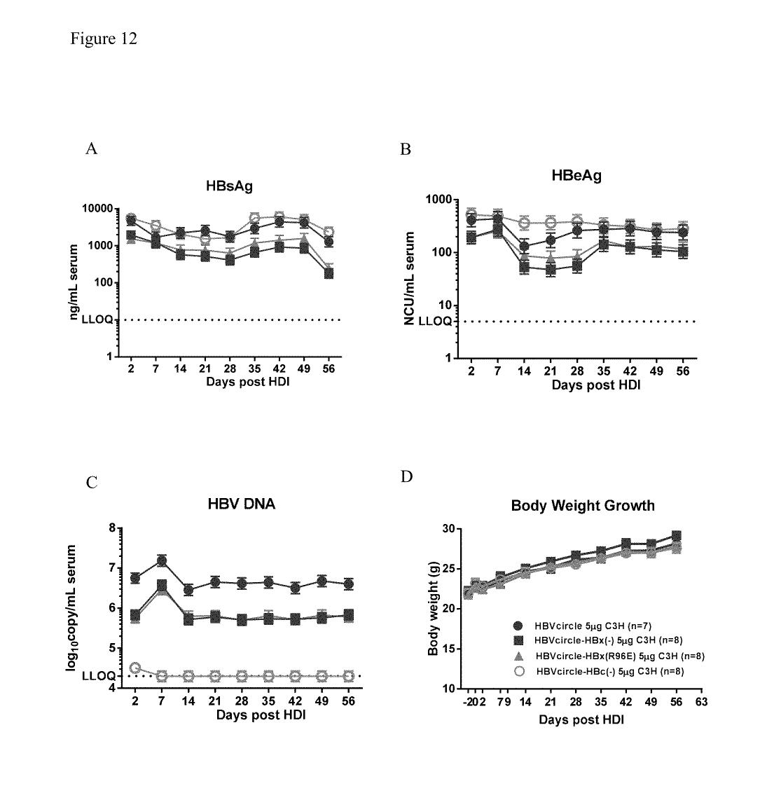

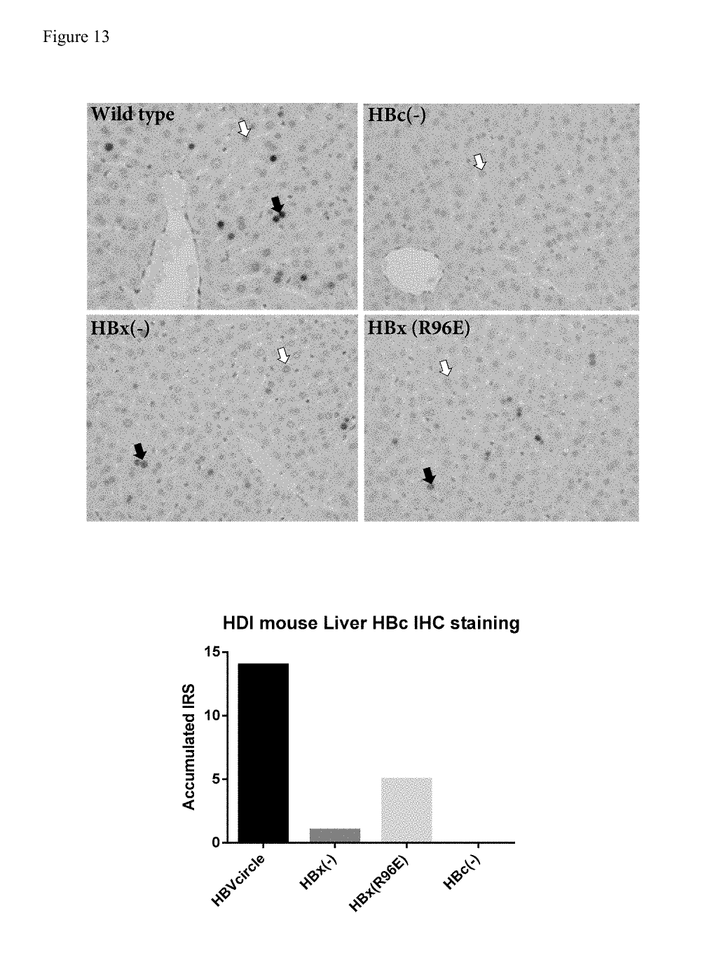

A series of HBVcircle mutants were generated and evaluated for its persistency in vivo. HBc deletion rendered the virus unable to replicate, and therefore caused undetectable HBV DNA in the serum. However, it did not affect the persistency of HBsAg and HBeAg. Similarly, HBx deletion (start codon mutation, SEQ ID No: 20) or R96E mutation (defective in DDB1 binding, SEQ ID No:21) (Leupin, et al., J Virol. 2005 April; 79(7): 4238-4245) did not affect HBV persistency either (FIG. 12). Decreased level of HBV replication was observed, as indicated by the reduced level of HBV DNA and antigens level in the serum compared to wildtype group (FIG. 12). In consistent with this finding, the mouse liver IHC staining results also demonstrated reduced HBc levels in the hepatocytes (Table 4 and FIG. 13).

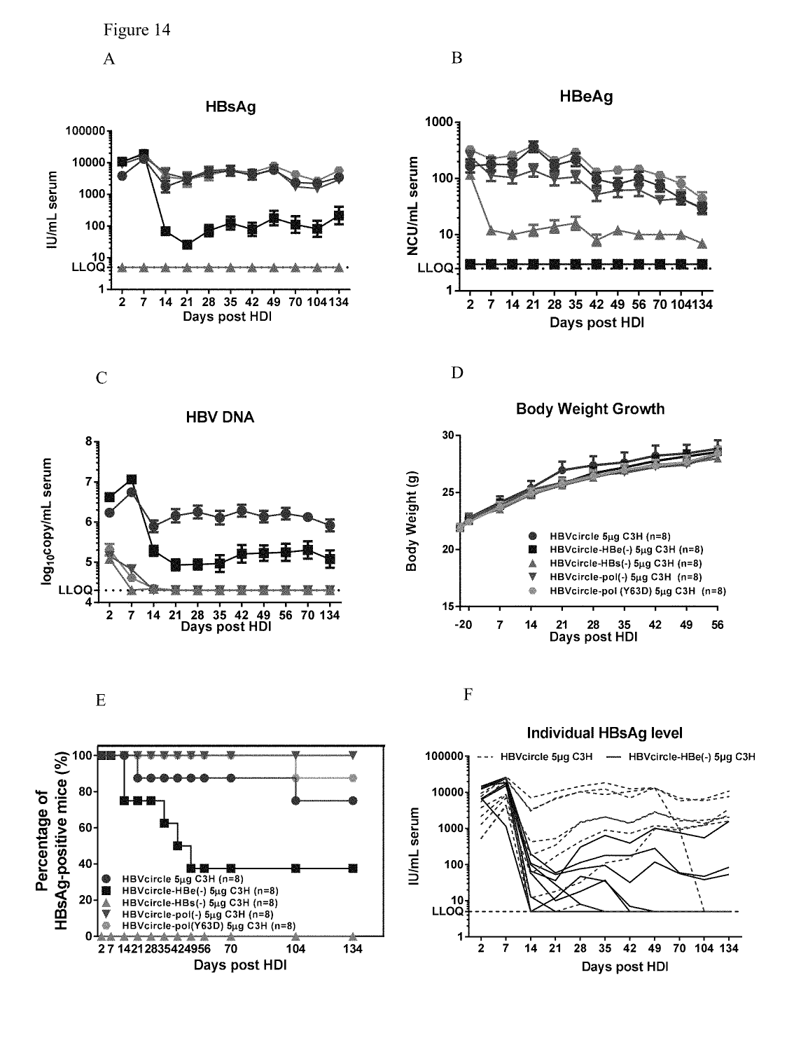

In a separate experiment, additional HBV mutants, including HBe(−), HBs(−), Pol(−), and Pol(Y63D) were tested. As shown in FIG. 14, HBe(−) mutant showed decreased persistency in HBsAg.

TABLE 4
IHC results quantification for FIG. 13

| | | Injected plasmid | | | | | | | |
|---|---|---|---|---|---|---|---|---|---|
| | | Wildtype | | HBx- | | HBx-R95E | | HBc- | |
| | | Animal ID | | | | | | | |
| | | 101 | 102 | 111 | 112 | 113 | 114 | 115 | 116 |
| Serum HBV | HBsAg | 3230 | 3480 | 1745 | 1197 | 3157 | 1820 | 2490 | 980 |
| markers at Day | (IU/ml) | | | | | | | | |
| 56⁽⁵⁾ | HBeAg | 316 | 299 | 201 | 96 | 152 | 67 | 297 | 123 |
| | (NCU/ml) | | | | | | | | |
| | DNA | 6.34 | 6.11 | 5.42 | 5.10 | 5.24 | 5.15 | 4.30 | 4.30 |
| | (Log10 | | | | | | | | |
| | copy/ml) | | | | | | | | |

| | | | | | | | | |
|---|---|---|---|---|---|---|---|---|
| Positive staining | 1/1/1/1/1 | 1/1/1/1/1 | 0/0/0/0/0 | 0/1/0/0/0 | 0/0/0/0/0 | 1/1/1/1/1 | 0/0/0/0/0 | 0/0/0/0/0 |
| percent⁽¹⁾ | | | | | | | | |
| Staining intensity⁽²⁾ | 1/1/2/2/2 | 1/1/1/2/1/ | 0/0/0/0/0 | 0/1/0/0/0 | 0/0/0/0/0 | 1/1/1/1/1 | 0/0/0/0/0 | 0/0/0/0/0 |
| IRS⁽³⁾ | 1/1/2/2/2 | 1/1/1/2/1 | 0/0/0/0/0 | 0/1/0/0/0 | 0/0/0/0/0 | 1/1/1/1/1 | 0/0/0/0/0 | 0/0/0/0/0 |
| Accumulated IRS⁽⁴⁾ | 8 | 6 | 0 | 1 | 0 | 5 | 0 | 0 |

⁽¹⁾Positive staining percent scores: 0 (negative); 1 (positive cells <25%); 2 (positive cells 25%~50%); 3 (positive cells, 50%~70%); 4 (positive cells >75%).
⁽²⁾Staining intensity from weak to strong: 0~3.
⁽³⁾IRS: positive staining percent scores × staining intensity scores.
⁽⁴⁾Accumulated IRS: sum of five replicates IRS.
⁽⁵⁾LLOQ for HBsAg is 10 IU/ml; LLOQ for HBeAg is 5 NCU/ml; LLOQ for HBV DNA is 4.30 Log10(copy/ml).

1. A recombinant HBV cccDNA, comprising HBV genome or the fragment or variant thereof and a site-hybrid insert.

2. The recombinant HBV cccDNA of claim 1, wherein the site-hybrid insert is attR site.

3. The recombinant HBV cccDNA of claim 1 or 2, wherein the attR site is located immediately preceding the starting codon of preS1 gene, and between the terminal protein domain and spacer of the polymerase gene.

4. The recombinant HBV cccDNA of any one of claims 1 to 3, wherein the attR site is located between the 2847 and 2848 positions of SEQ ID NO:3.

5. The recombinant HBV cccDNA of anyone of claims 1 to 4, wherein HBV genome is the full length genome, particularly the genotype B or genotype D genome, more particularly the genome specified in GeneBank JN664917.1, X02496, AY217370, AY220698, GQ205440 or HPBHBVAA, most particularly the genome represented by SEQ ID NO:3, SEQ ID NO:22 or SEQ ID NO:23; or is the over length genome, particularly 1.1unit or 1.3unit genome of genotype D, more particularly 1.3 unit genome represented by SEQ ID NO:9.

6. The recombinant HBV cccDNA of any one of claims 1 to 5, wherein the fragment of the HBV genome in the recombinant HBV cccDNA can replicate or express the genes encoding envelope proteins, core/precore proteins, x protein and/or polymerase protein of HBV.

7. The recombinant HBV cccDNA of claim 1, the sequence of which is listed in SEQ ID NO:2.

8. The recombinant HBV cccDNA of anyone of claims 1 to 6, for transfecting a cell line or primary cell.

9. The recombinant HBV cccDNA of claim 8, wherein the cell line is the cell line from hepatic cells, particularly those from hepatocyte, more particularly HepG2 or HepaRG, or the primary cell is primary hepatic cells, particularly primary hepatocyte.

10. The recombinant HBV cccDNA of anyone of claims 1 to 9, for anti-HBV drug evaluation.

11. The recombinant HBV cccDNA of claim 9, wherein the anti-HBV drug is ETV, HAP 12, HAP 2, Pegasys or R848.

12. The recombinant HBV cccDNA of anyone of claims 1 to 11, for use in the method to establish a cccDNA based HBV animal model, wherein the method comprises delivering said recombinant HBV cccDNA into an animal.

13. The recombinant HBV cccDNA of anyone of claims 1 to 12, wherein the established animal model express HBV antigens for at least 30 days in the hepatocytes, particularly at least 37 days, 42 days, 44 days, 49 days, 51 days, 56 days, 70 days, 104 days, 120 days or 134 days in the hepatocytes.

14. The recombinant HBV cccDNA of anyone of claims 1 to 13, wherein the animal is immunocompetent with functional innate and adaptive immunity.

15. The recombinant HBV cccDNA of anyone of claims 1 to 14, wherein the animal is mouse, particularly the mouse is C3H/HeN or CBA/J mouse.

16. The recombinant HBV cccDNA of anyone of claims 1 to 15, wherein the recombinant HBV cccDNA is delivered into the animal via hydrodynamic injection.

17. The composition or kit comprising the recombinant HBV cccDNA of anyone of claims 1 to 16.

18. A method to prepare recombinant HBV cccDNA of any one of claims 1 to 17 comprising the following steps:
a) HBV genome or the fragment or variant thereof is inserted in and flanked by recombination substrate sites of minicircle DNA producing parental vector to form a parental HBVcircle construct;
b) the parental HBVcircle construct is transformed into the minicircle producer to generate recombinant HBV cccDNA via site-specific recombination.

19. The method of claim 18, wherein minicircle producer is microorganism, preferable bacterium, more particularly Escherichia sp., most particularly E. coli, particularly strain ZYCY10P3S2T.

20. The method of claim 18 or 19, wherein the minicircle DNA producing parental vector contains recombination substrate sites, particularly the recombination substrate sites specific to the recombinase, more particularly specific to integrase, most particularly integrases of ΦC31, R4, TP901-1, ΦBT1, Bxb1, RV-1, AA118, U153, ΦFC1.

21. The method of any one of claims 18 to 20, wherein the recombination substrate sites are attP and attB.

22. The method according to any one of claims 18 to 21, wherein the minicircle DNA producing parental vector is pMC.CMV-MCS-SV40polyA vector.

23. The method according to any one of claims 18 to 22, wherein the DNA sequence of parental HBVcircle construct is listed as SEQ ID NO:1.

24. The use of the recombinant HBV cccDNA of anyone of claims 1 to 16 or composition or kit of claim 17 in the evaluation of a medicament for the treatment of hepatitis B virus infection.


SEQUENCE LISTING (23 sequences)

SEQ ID NO: 1 - DNA, 7220 bp, Hepatitis B virus
acattaccctgttatccctagatgacattaccctgttatcccagatgacattaccctgttatccctagatgacattaccctgttatccctagatgacatttaccctgttatccctagatgacattaccctgttatcccagatgacattaccctgttatccctagatacattaccctgttatcccagatgacataccctgttatccctagatgacattaccctgttatcccagatgacattaccctgttatccctagatacattaccctgttatcccagatgacataccctgttatccctagatgacattaccctgttatcccagatgacattaccctgttatccctagatacattaccctgttatcccagatgacataccctgttatccctagatgacattaccctgttatcccagatgacattaccctgttatccctagatacattaccctgttatcccagatgacataccctgttatccctagatgacattaccctgttatcccagatgacattaccctgttatccctagatacattaccctgttatcccagatgacataccctgttatccctagatgacattaccctgttatcccagatgacattaccctgttatccctagatacattaccctgttatcccagatgacataccctgttatccctagatgacattaccctgttatcccagataaactcaatgatgatgatgatgatggtcgagactcagcggccgcggtgccagggcgtgcccttgggctccccgggcgcgaccatggggcagaatctttccaccagcaatcctctgggattctttcccgaccaccagttggatccagccttcagagcaaacaccgcaaatccagattgggacttcaatcccaacaaggacacctggccagacgccaacaaggtaggagctggagcattcgggctgggtttcaccccaccgcacggaggccttttggggtggagccctcaggctcagggcatactacaaactttgccagcaaatccgcctcctgcctccaccaatcgccagtcaggaaggcagcctaccccgctgtctccacctttgagaaacactcatcctcaggccatgcagtggaattccacaaccttccaccaaactctgcaagatcccagagtgagaggcctgtatttccctgctggtggctccagttcaggaacagtaaaccctgttctgactactgcctctcccttatcgtcaatcttctcgaggattggggaccctgcgctgaacatggagaacatcacatcaggattcctaggaccccttctcgtgttacaggcggggtttttcttgttgacaagaatcctcacaataccgcagagtctagactcgtggtggacttctctcaattttctagggggaactaccgtgtgtcttggccaaaattcgcagtccccaacctccaatcactcaccaacctcttgtcctccaacttgtcctggttatcgctggatgtgtctgcggcgttttatcatcttcctcttcatcctgctgctatgcctcatcttcttgttggttcttctggactatcaaggtatgttgcccgtttgtcctctaattccaggatcctcaacaaccagcacgggaccatgccggacctgcatgactactgctcaaggaacctctatgtatccctcctgttgctgtaccaaaccttcggacggaaattgcacctgtattcccatcccatcatcctgggctttcggaaaattcctatgggagtgggcctcagcccgtttctcctggctcagtttactagtgccatttgttcagtggttcgtagggctttcccccactgtttggctttcagttatatggatgatgtggtattgggggccaagtctgtacagcatcttgagtccctttttaccgctgttaccaattttcttttgtctttgggtatacatttaaaccctaacaaaacaaagagatggggttactctctaaattttatgggttatgtcattggatgttatgggtccttgccacaagaacacatcatacaaaaaatcaaagaatgttttagaaaacttcctattaacaggcctattgattggaaagtatgtcaacgaattgtgggtcttttgggttttgctgccccttttacacaatgtggttatcctgcgttgatgcctttgtatgcatgtattcaatctaagcaggctttcactttctcgccaacttacaaggcctttctgtgtaaacaatacctgaacctttaccccgttgcccggcaacggccaggtctgtgccaagtgtttgctgacgcaacccccactggctggggcttggtcatgggccatcagcgcatgcgtggaaccttttcggctcctctgccgatccatactgcggaactcctagccgcttgttttgctcgcagcaggtctggagcaaacattatcgggactgataactctgttgtcctatcccgcaaatatacatcgtttccatggctgctaggctgtgctgccaactggatcctgcgcgggacgtcctttgtttacgtcccgtcggcgctgaatcctgcggacgacccttctcggggtcgcttgggactctctcgtccccttctccgtctgccgttccgaccgaccacggggcgcacctctctttacgcggactccccgtctgtgccttctcatctgccggaccgtgtgcacttcgcttcacctctgcacgtcgcatggagaccaccgtgaacgcccaccaaatattgcccaaggtcttacataagaggactcttggactctcagcaatgtcaacgaccgaccttgaggcatacttcaaagactgtttgtttaaagactgggaggagttgggggaggagattaggttaaaggtctttgtactaggaggctgtaggcataaattggtctgcgcaccagcaccatgcaactttttcacctctgcctaatcatctcttgttcatgtcctactgttcaagcctccaagctgtgccttgggtggctttggggcatggacatcgacccttataaagaatttggagctactgtggagttactctcgtttttgccttctgacttctttccttcagtacgagatcttctagataccgcctcagctctgtatcgggaagccttagagtctcctgagcattgttcacctcaccatactgcactcaggcaagcaattctttgctggggggaactaatgactctagctacctgggtgggtgttaatttggaagatccagcgtctagagacctagtagtcagttatgtcaacactaatatgggcctaaagttcaggcaactcttgtggtttcacatttcttgtctcacttttggaagagaaacagttatagagtatttggtgtctttcggagtgtggattcgcactcctccagcttatagaccaccaaatgcccctatcctatcaacacttccggagactactgttgttagacgacgaggcaggtcccctagaagaagaactccctcgcctcgcagacgaaggtctcaatcgccgcgtcgcagaagatctcaatctcgggaatctcaatgttagtattccttggactcataaggtggggaactttactgggctttattcttctactgtacctgtctttaatcctcattggaaaacaccatcttttcctaatatacatttacaccaagacattatcaaaaaatgtgaacagtttgtaggcccactcacagttaatgagaaaagaagattgcaattgattatgcctgccaggttttatccaaaggttaccaaatatttaccattggataagggtattaaaccttattatccagaacatctagttaatcattacttccaaactagacactatttacacactctatggaaggcgggtatattatataagagagaaacaacacatagcgcctcattttgtgggtcaccatattcttgggaacaagatctacagcccccaactggggtaacctttgagttctctcagttgggggtaatcagcatcatgatgtggtaccacatcatgatgctgattataagaatgcggccgccacactctagtggatctcgagttaataattcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgctcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgtagatgacatggagatcctgccccggcacttcgcccaatagcagccagtcccttcccgcttcagtgacaacgtcgagcacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagagcttgatcccctgcgccatcagatccttggcggcgagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatccggggtcagcaccgtttctgcggactggctttctacgtgctcgaggggggccaaacggtctccagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttttgtttatttttctaaatacattcaaatatgtatccgctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagcagatcaattcgcgcgcgaaggcgaagcggcatgcataatgtgcctgtcaaatggacgaagcagggattctgcaaaccctatgctactccgtcaagccgtcaattgtctgattcgttaccaattatgacaacttgacggctacatcattcactttttcttcacaaccggcacggaactcgctcgggctggccccggtgcattttttaaatacccgcgagaaatagagttgatcgtcaaaaccaacattgcgaccgacggtggcgataggcatccgggtggtgctcaaaagcagcttcgcctggctgatacgttggtcctcgcgccagcttaagacgctaatccctaactgctggcggaaaagatgtgacagacgcgacggcgacaagcaaacatgctgtgcgacgctggcgat

SEQ ID NO: 2 - DNA, 3221 bp, Hepatitis B virus
cccgggcgcgaccatggggcagaatctttccaccagcaatcctctgggattctttcccgaccaccagttggatccagccttcagagcaaacaccgcaaatccagattgggacttcaatcccaacaaggacacctggccagacgccaacaaggtaggagctggagcattcgggctgggtttcaccccaccgcacggaggccttttggggtggagccctcaggctcagggcatactacaaactttgccagcaaatccgcctcctgcctccaccaatcgccagtcaggaaggcagcctaccccgctgtctccacctttgagaaacactcatcctcaggccatgcagtggaattccacaaccttccaccaaactctgcaagatcccagagtgagaggcctgtatttccctgctggtggctccagttcaggaacagtaaaccctgttctgactactgcctctcccttatcgtcaatcttctcgaggattggggaccctgcgctgaacatggagaacatcacatcaggattcctaggaccccttctcgtgttacaggcggggtttttcttgttgacaagaatcctcacaataccgcagagtctagactcgtggtggacttctctcaattttctagggggaactaccgtgtgtcttggccaaaattcgcagtccccaacctccaatcactcaccaacctcttgtcctccaacttgtcctggttatcgctggatgtgtctgcggcgttttatcatcttcctcttcatcctgctgctatgcctcatcttcttgttggttcttctggactatcaaggtatgttgcccgtttgtcctctaattccaggatcctcaacaaccagcacgggaccatgccggacctgcatgactactgctcaaggaacctctatgtatccctcctgttgctgtaccaaaccttcggacggaaattgcacctgtattcccatcccatcatcctgggctttcggaaaattcctatgggagtgggcctcagcccgtttctcctggctcagtttactagtgccatttgttcagtggttcgtagggctttcccccactgtttggctttcagttatatggatgatgtggtattgggggccaagtctgtacagcatcttgagtccctttttaccgctgttaccaattttcttttgtctttgggtatacatttaaaccctaacaaaacaaagagatggggttactctctaaattttatgggttatgtcattggatgttatgggtccttgccacaagaacacatcatacaaaaaatcaaagaatgttttagaaaacttcctattaacaggcctattgattggaaagtatgtcaacgaattgtgggtcttttgggttttgctgccccttttacacaatgtggttatcctgcgttgatgcctttgtatgcatgtattcaatctaagcaggctttcactttctcgccaacttacaaggcctttctgtgtaaacaatacctgaacctttaccccgttgcccggcaacggccaggtctgtgccaagtgtttgctgacgcaacccccactggctggggcttggtcatgggccatcagcgcatgcgtggaaccttttcggctcctctgccgatccatactgcggaactcctagccgcttgttttgctcgcagcaggtctggagcaaacattatcgggactgataactctgttgtcctatcccgcaaatatacatcgtttccatggctgctaggctgtgctgccaactggatcctgcgcgggacgtcctttgtttacgtcccgtcggcgctgaatcctgcggacgacccttctcggggtcgcttgggactctctcgtccccttctccgtctgccgttccgaccgaccacggggcgcacctctctttacgcggactccccgtctgtgccttctcatctgccggaccgtgtgcacttcgcttcacctctgcacgtcgcatggagaccaccgtgaacgcccaccaaatattgcccaaggtcttacataagaggactcttggactctcagcaatgtcaacgaccgaccttgaggcatacttcaaagactgtttgtttaaagactgggaggagttgggggaggagattaggttaaaggtctttgtactaggaggctgtaggcataaattggtctgcgcaccagcaccatgcaactttttcacctctgcctaatcatctcttgttcatgtcctactgttcaagcctccaagctgtgccttgggtggctttggggcatggacatcgacccttataaagaatttggagctactgtggagttactctcgtttttgccttctgacttctttccttcagtacgagatcttctagataccgcctcagctctgtatcgggaagccttagagtctcctgagcattgttcacctcaccatactgcactcaggcaagcaattctttgctggggggaactaatgactctagctacctgggtgggtgttaatttggaagatccagcgtctagagacctagtagtcagttatgtcaacactaatatgggcctaaagttcaggcaactcttgtggtttcacatttcttgtctcacttttggaagagaaacagttatagagtatttggtgtctttcggagtgtggattcgcactcctccagcttatagaccaccaaatgcccctatcctatcaacacttccggagactactgttgttagacgacgaggcaggtcccctagaagaagaactccctcgcctcgcagacgaaggtctcaatcgccgcgtcgcagaagatctcaatctcgggaatctcaatgttagtattccttggactcataaggtggggaactttactgggctttattcttctactgtacctgtctttaatcctcattggaaaacaccatcttttcctaatatacatttacaccaagacattatcaaaaaatgtgaacagtttgtaggcccactcacagttaatgagaaaagaagattgcaattgattatgcctgccaggttttatccaaaggttaccaaatatttaccattggataagggtattaaaccttattatccagaacatctagttaatcattacttccaaactagacactatttacacactctatggaaggcgggtatattatataagagagaaacaacacatagcgcctcattttgtgggtcaccatattcttgggaacaagatctacagcccccaactggggtaacctttgggctc

SEQ ID NO: 3 - DNA, 3182 bp, Hepatitis B virus
ttccacaaccttccaccaagctcctgcagatcccagagtgagaggcctgtatttccctgctggtggctccagttcaggaacagtaaaccctgttctgactactgcctctcccttatcgtcaatcttctcgaggattggggaccctgcgctgaacatggagaacatcacatcaggattcctaggaccccttctcgtgttacaggcggggtttttcttgttgacaagaatcctcacaataccgcagagtctagactcgtggtggacttctctcaattttctagggggaactaccgtgtgtcttggccaaaattcgcagtccccaacctccaatcactcaccaacctcttgtcctccaacttgtcctggttatcgctggatgtgtctgcggcgttttatcatcttcctcttcatcctgctgctatgcctcatcttcttgttggttcttctggactatcaaggtatgttgcccgtttgtcctctaattccaggatcctcaacaaccagcacgggaccatgccggacctgcatgactactgctcaaggaacctctatgtatccctcctgttgctgtaccaaaccttcggacggaaattgcacctgtattcccatcccatcatcctgggctttcggaaaattcctatgggagtgggcctcagcccgtttctcctggctcagtttactagtgccatttgttcagtggttcgtagggctttcccccactgtttggctttcagttatatggatgatgtggtattgggggccaagtctgtacagcatcttgagtccctttttaccgctgttaccaattttcttttgtctttgggtatacatttaaaccctaacaaaacaaagagatggggttactctctaaattttatgggttatgtcattggatgttatgggtccttgccacaagaacacatcatacaaaaaatcaaagaatgttttagaaaacttcctattaacaggcctattgattggaaagtatgtcaacgaattgtgggtcttttgggttttgctgccccttttacacaatgtggttatcctgcgttgatgcctttgtatgcatgtattcaatctaagcaggctttcactttctcgccaacttacaaggcctttctgtgtaaacaatacctgaacctttaccccgttgcccggcaacggccaggtctgtgccaagtgtttgctgacgcaacccccactggctggggcttggtcatgggccatcagcgcatgcgtggaaccttttcggctcctctgccgatccatactgcggaactcctagccgcttgttttgctcgcagcaggtctggagcaaacattatcgggactgataactctgttgtcctatcccgcaaatatacatcgtttccatggctgctaggctgtgctgccaactggatcctgcgcgggacgtcctttgtttacgtcccgtcggcgctgaatcctgcggacgacccttctcggggtcgcttgggactctctcgtccccttctccgtctgccgttccgaccgaccacggggcgcacctctctttacgcggactccccgtctgtgccttctcatctgccggaccgtgtgcacttcgcttcacctctgcacgtcgcatggagaccaccgtgaacgcccaccaaatattgcccaaggtcttacataagaggactcttggactctcagcaatgtcaacgaccgaccttgaggcatacttcaaagactgtttgtttaaagactgggaggagttgggggaggagattaggttaaaggtctttgtactaggaggctgtaggcataaattggtctgcgcaccagcaccatgcaactttttcacctctgcctaatcatctcttgttcatgtcctactgttcaagcctccaagctgtgccttgggtggctttggggcatggacatcgacccttataaagaatttggagctactgtggagttactctcgtttttgccttctgacttctttccttcagtacgagatcttctagataccgcctcagctctgtatcgggaagccttagagtctcctgagcattgttcacctcaccatactgcactcaggcaagcaattctttgctggggggaactaatgactctagctacctgggtgggtgttaatttggaagatccagcgtctagagacctagtagtcagttatgtcaacactaatatgggcctaaagttcaggcaactcttgtggtttcacatttcttgtctcacttttggaagagaaacagttatagagtatttggtgtctttcggagtgtggattcgcactcctccagcttatagaccaccaaatgcccctatcctatcaacacttccggagactactgttgttagacgacgaggcaggtcccctagaagaagaactccctcgcctcgcagacgaaggtctcaatcgccgcgtcgcagaagatctcaatctcgggaatctcaatgttagtattccttggactcataaggtggggaactttactgggctttattcttctactgtacctgtctttaatcctcattggaaaacaccatcttttcctaatatacatttacaccaagacattatcaaaaaatgtgaacagtttgtaggcccactcacagttaatgagaaaagaagattgcaattgattatgcctgccaggttttatccaaaggttaccaaatatttaccattggataagggtattaaaccttattatccagaacatctagttaatcattacttccaaactagacactatttacacactctatggaaggcgggtatattatataagagagaaacaacacatagcgcctcattttgtgggtcaccatattcttgggaacaagatctacagcatggggcagaatctttccaccagcaatcctctgggattctttcccgaccaccagttggatccagccttcagagcaaacaccgcaaatccagattgggacttcaatcccaacaaggacacctggccagacgccaacaaggtaggagctggagcattcgggctgggtttcaccccaccgcacggaggccttttggggtggagccctcaggctcagggcatactacaaactttgccagcaaatccgcctcctgcctccaccaatcgccagtcaggaaggcagcctaccccgctgtctccacctttgagaaacactcatcctcaggccatgcagtggaa

SEQ ID NO: 4 - DNA, 39 bp, Bacteriophage phi-C31
ccccaactggggtaacctttgggctccccgggcgcgacc

SEQ ID NO: 5 - DNA, 36 bp, Bacteriophage phi-C31
cccaactggggtaacctttgagttctctagttgggg

SEQ ID NO: 6 - DNA, 35 bp, Bacteriophage phi-C31
ggtgccagggcgtgcccttgggctccccgggcgcg

SEQ ID NO: 7 - DNA, 6805 bp, Hepatitis B virus
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcaatctaagcaggctttcactttctcgccaacttacaaggcctttctgtgtaaacaatacctgaacctttaccccgttgcccggcaacggccaggtctgtgccaagtgtttgctgacgcaacccccactggctggggcttggtcatgggccatcagcgcatgcgtggaaccttttcggctcctctgccgatccatactgcggaactcctagccgcttgttttgctcgcagcaggtctggagcaaacattatcgggactgataactctgttgtcctatcccgcaaatatacatcgtttccatggctgctaggctgtgctgccaactggatcctgcgcgggacgtcctttgtttacgtcccgtcggcgctgaatcctgcggacgacccttctcggggtcgcttgggactctctcgtccccttctccgtctgccgttccgaccgaccacggggcgcacctctctttacgcggactccccgtctgtgccttctcatctgccggaccgtgtgcacttcgcttcacctctgcacgtcgcatggagaccaccgtgaacgcccaccaaatattgcccaaggtcttacataagaggactcttggactctcagcaatgtcaacgaccgaccttgaggcatacttcaaagactgtttgtttaaagactgggaggagttgggggaggagattaggttaaaggtctttgtactaggaggctgtaggcataaattggtctgcgcaccagcaccatgcaactttttcacctctgcctaatcatctcttgttcatgtcctactgttcaagcctccaagctgtgccttgggtggctttggggcatggacatcgacccttataaagaatttggagctactgtggagttactctcgtttttgccttctgacttctttccttcagtacgagatcttctagataccgcctcagctctgtatcgggaagccttagagtctcctgagcattgttcacctcaccatactgcactcaggcaagcaattctttgctggggggaactaatgactctagctacctgggtgggtgttaatttggaagatccagcgtctagagacctagtagtcagttatgtcaacactaatatgggcctaaagttcaggcaactcttgtggtttcacatttcttgtctcacttttggaagagaaacagttatagagtatttggtgtctttcggagtgtggattcgcactcctccagcttatagaccaccaaatgcccctatcctatcaacacttccggagactactgttgttagacgacgaggcaggtcccctagaagaagaactccctcgcctcgcagacgaaggtctcaatcgccgcgtcgcagaagatctcaatctcgggaatctcaatgttagtattccttggactcataaggtggggaactttactgggctttattcttctactgtacctgtctttaatcctcattggaaaacaccatcttttcctaatatacatttacaccaagacattatcaaaaaatgtgaacagtttgtaggcccactcacagttaatgagaaaagaagattgcaattgattatgcctgccaggttttatccaaaggttaccaaatatttaccattggataagggtattaaaccttattatccagaacatctagttaatcattacttccaaactagacactatttacacactctatggaaggcgggtatattatataagagagaaacaacacatagcgcctcattttgtgggtcaccatattcttgggaacaagatctacagcatggggcagaatctttccaccagcaatcctctgggattctttcccgaccaccagttggatccagccttcagagcaaacaccgcaaatccagattgggacttcaatcccaacaaggacacctggccagacgccaacaaggtaggagctggagcattcgggctgggtttcaccccaccgcacggaggccttttggggtggagccctcaggctcagggcatactacaaactttgccagcaaatccgcctcctgcctccaccaatcgccagtcaggaaggcagcctaccccgctgtctccacctttgagaaacactcatcctcaggccatgcagtggaattccacaaccttccaccaaactctgcaagatcccagagtgagaggcctgtatttccctgctggtggctccagttcaggaacagtaaaccctgttctgactactgcctctcccttatcgtcaatcttctcgaggattggggaccctgcgctgaacatggagaacatcacatcaggattcctaggaccccttctcgtgttacaggcggggtttttcttgttgacaagaatcctcacaataccgcagagtctagactcgtggtggacttctctcaattttctagggggaactaccgtgtgtcttggccaaaattcgcagtccccaacctccaatcactcaccaacctcttgtcctccaacttgtcctggttatcgctggatgtgtctgcggcgttttatcatcttcctcttcatcctgctgctatgcctcatcttcttgttggttcttctggactatcaaggtatgttgcccgtttgtcctctaattccaggatcctcaacaaccagcacgggaccatgccggacctgcatgactactgctcaaggaacctctatgtatccctcctgttgctgtaccaaaccttcggacggaaattgcacctgtattcccatcccatcatcctgggctttcggaaaattcctatgggagtgggcctcagcccgtttctcctggctcagtttactagtgccatttgttcagtggttcgtagggctttcccccactgtttggctttcagttatatggatgatgtggtattgggggccaagtctgtacagcatcttgagtccctttttaccgctgttaccaattttcttttgtctttgggtatacatttaaaccctaacaaaacaaagagatggggttactctctaaattttatgggttatgtcattggatgttatgggtccttgccacaagaacacatcatacaaaaaatcaaagaatgttttagaaaacttcctattaacaggcctattgattggaaagtatgtcaacgaattgtgggtcttttgggttttgctgccccttttacacaatgtggttatcctgcgttgatgcctttgtatgcatgtattcaatctaagcaggctttcactttctcgccaacttacaaggcctttctgtgtaaacaatacctgaacctttaccccgttgcccggcaacggccaggtctgtgccaagtgtttgctgacgcaacccccactggctggggcttggtcatgggccatcagcgcatgcgtggaaccttttcggctcctctgccgatccatactgcggaactcctagccgcttgttttgctcgcagcaggtctggagcaaacattatcgggactgataactctgttgtcctatcccgcaaatatacatcgtttccatggctgctaggctgtgctgccaactggatcctgcgcgggacgtcctttgtttacgtcccgtcggcgctgaatcctgcggacgacccttctcggggtcgcttgggactctctcgtccccttctccgtctgccgttccgaccgaccacggggcgcacctctctttacgcggactccccgtctgtgccttctcatctgccggaccgtgtgcacttcgcttcacctctgcacgtcgcatggagaccaccgtgaacgcccaccaaatattgcccaaggtcttacataagaggactcttggactctcagcaatgtcaacgaccgaccttgaggcatacttcaaagactgtttgtttaaagactgggaggagttgggggaggagattaggttaaaggtctttgtactaggaggctgtaggcataaattggtctgcgcaccagcaccatgcaactttttcacctctgcctaatcatctcttgttcatgtcctactgttcaagcctccaagctgtgccttgggtggctttggggcatggacatcgacccttataaagaatttggagctactgtggagttactctcgtttttgccttctgacttctttccttcagtacgagatccactagttctagagcggccgccaccgcggtggagctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgaagcttagactgcagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 8 - DNA, 9170 bp, Hepatitis B virus
tcttctctcatccgccaaaacagccaagctggagaccgtttgacattaccctgttatccctagatacattaccctgttatcccagatgacataccctgttatccctagatgacattaccctgttatcccagatgacattaccctgttatccctagatacattaccctgttatcccagatgacataccctgttatccctagatgacattaccctgttatcccagatgacattaccctgttatccctagatacattaccctgttatcccagatgacataccctgttatccctagatgacattaccctgttatcccagatgacattaccctgttatccctagatacattaccctgttatcccagatgacataccctgttatccctagatgacattaccctgttatcccagatgacattaccctgttatccctagatacattaccctgttatcccagatgacataccctgttatccctagatgacattaccctgttatcccagatgacattaccctgttatccctagatacattaccctgttatcccagatgacataccctgttatccctagatgacattaccctgttatcccagatgacattaccctgttatccctagatacattaccctgttatcccagatgacataccctgttatccctagatgacattaccctgttatcccagatgacattaccctgttatccctagatacattaccctgttatcccagatgacataccctgttatccctagatgacattaccctgttatcccagataaactcaatgatgatgatgatgatggtcgagactcagcggccgcggtgccagggcgtgcccttgggctccccgggcgcgactagtgaattgatactagtattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtttatataagcagagctcgtttagtgaaccgtcagatcgcctggagacgccatccacgctgttttgacctccatagaagattctagagtcgacaccagcaccatgcaactttttcacctctgcctaatcatctcttgttcatgtcctactgttcaagcctccaagctgtgccttgggtggctttggggcatggacatcgacccttataaagaatttggagctactgtggagttactctcgtttttgccttctgacttctttccttcagtacgagatcttctagataccgcctcagctctgtatcgggaagccttagagtctcctgagcattgttcacctcaccatactgcactcaggcaagcaattctttgctggggggaactaatgactctagctacctgggtgggtgttaatttggaagatccagcgtctagagacctagtagtcagttatgtcaacactaatatgggcctaaagttcaggcaactcttgtggtttcacatttcttgtctcacttttggaagagaaacagttatagagtatttggtgtctttcggagtgtggattcgcactcctccagcttatagaccaccaaatgcccctatcctatcaacacttccggagactactgttgttagacgacgaggcaggtcccctagaagaagaactccctcgcctcgcagacgaaggtctcaatcgccgcgtcgcagaagatctcaatctcgggaatctcaatgttagtattccttggactcataaggtggggaactttactgggctttattcttctactgtacctgtctttaatcctcattggaaaacaccatcttttcctaatatacatttacaccaagacattatcaaaaaatgtgaacagtttgtaggcccactcacagttaatgagaaaagaagattgcaattgattatgcctgccaggttttatccaaaggttaccaaatatttaccattggataagggtattaaaccttattatccagaacatctagttaatcattacttccaaactagacactatttacacactctatggaaggcgggtatattatataagagagaaacaacacatagcgcctcattttgtgggtcaccatattcttgggaacaagatctacagcatggggcagaatctttccaccagcaatcctctgggattctttcccgaccaccagttggatccagccttcagagcaaacaccgcaaatccagattgggacttcaatcccaacaaggacacctggccagacgccaacaaggtaggagctggagcattcgggctgggtttcaccccaccgcacggaggccttttggggtggagccctcaggctcagggcatactacaaactttgccagcaaatccgcctcctgcctccaccaatcgccagtcaggaaggcagcctaccccgctgtctccacctttgagaaacactcatcctcaggccatgcagtggaattccacaaccttccaccaaactctgcaagatcccagagtgagaggcctgtatttccctgctggtggctccagttcaggaacagtaaaccctgttctgactactgcctctcccttatcgtcaatcttctcgaggattggggaccctgcgctgaacatggagaacatcacatcaggattcctaggaccccttctcgtgttacaggcggggtttttcttgttgacaagaatcctcacaataccgcagagtctagactcgtggtggacttctctcaattttctagggggaactaccgtgtgtcttggccaaaattcgcagtccccaacctccaatcactcaccaacctcttgtcctccaacttgtcctggttatcgctggatgtgtctgcggcgttttatcatcttcctcttcatcctgctgctatgcctcatcttcttgttggttcttctggactatcaaggtatgttgcccgtttgtcctctaattccaggatcctcaacaaccagcacgggaccatgccggacctgcatgactactgctcaaggaacctctatgtatccctcctgttgctgtaccaaaccttcggacggaaattgcacctgtattcccatcccatcatcctgggctttcggaaaattcctatgggagtgggcctcagcccgtttctcctggctcagtttactagtgccatttgttcagtggttcgtagggctttcccccactgtttggctttcagttatatggatgatgtggtattgggggccaagtctgtacagcatcttgagtccctttttaccgctgttaccaattttcttttgtctttgggtatacatttaaaccctaacaaaacaaagagatggggttactctctaaattttatgggttatgtcattggatgttatgggtccttgccacaagaacacatcatacaaaaaatcaaagaatgttttagaaaacttcctattaacaggcctattgattggaaagtatgtcaacgaattgtgggtcttttgggttttgctgccccttttacacaatgtggttatcctgcgttgatgcctttgtatgcatgtattcaatctaagcaggctttcactttctcgccaacttacaaggcctttctgtgtaaacaatacctgaacctttaccccgttgcccggcaacggccaggtctgtgccaagtgtttgctgacgcaacccccactggctggggcttggtcatgggccatcagcgcatgcgtggaaccttttcggctcctctgccgatccatactgcggaactcctagccgcttgttttgctcgcagcaggtctggagcaaacattatcgggactgataactctgttgtcctatcccgcaaatatacatcgtttccatggctgctaggctgtgctgccaactggatcctgcgcgggacgtcctttgtttacgtcccgtcggcgctgaatcctgcggacgacccttctcggggtcgcttgggactctctcgtccccttctccgtctgccgttccgaccgaccacggggcgcacctctctttacgcggactccccgtctgtgccttctcatctgccggaccgtgtgcacttcgcttcacctctgcacgtcgcatggagaccaccgtgaacgcccaccaaatattgcccaaggtcttacataagaggactcttggactctcagcaatgtcaacgaccgaccttgaggcatacttcaaagactgtttgtttaaagactgggaggagttgggggaggagattaggttaaaggtctttgtactaggaggctgtaggcataaattggtctgcgcaccagcaccatgcaactttttcacctctgcctaatcatctcttgttcatgtcctactgttcaagcctccaagctgtgccttgggtggctttggggcatggacatcgacccttataaagaatttggagctactgtggagttactctcgtttttgccttctgacttctttccttcagtacgagatcttgctagctcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggtacctttaagaccaatgacttacaaggcagctgtagatcttagccactttttaaaagaaaaggggggactggaagggctaattcactcccaacgaagataagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagacttttgcagatcgacccatgggggcccgccccaactggggtaacctttgagttctctcagttgggggtaatcagcatcatgatgtggtaccacatcatgatgctgattataagaatgcggccgccacactctagtggatctcgagttaataattcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgctcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgtagatgacatggagatcctgccccggcacttcgcccaatagcagccagtcccttcccgcttcagtgacaacgtcgagcacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagagcttgatcccctgcgccatcagatccttggcggcgagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatccggggtcagcaccgtttctgcggactggctttctacgtgctcgaggggggccaaacggtctccagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttttgtttatttttctaaatacattcaaatatgtatccgctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagcagatcaattcgcgcgcgaaggcgaagcggcatgcataatgtgcctgtcaaatggacgaagcagggattctgcaaaccctatgctactccgtcaagccgtcaattgtctgattcgttaccaattatgacaacttgacggctacatcattcactttttcttcacaaccggcacggaactcgctcgggctggccccggtgcattttttaaatacccgcgagaaatagagttgatcgtcaaaaccaacattgcgaccgacggtggcgataggcatccgggtggtgctcaaaagcagcttcgcctggctgatacgttggtcctcgcgccagcttaagacgctaatccctaactgctggcggaaaagatgtgacagacgcgacggcgacaagcaaacatgctgtgcgacgctggcgat

SEQ ID NO: 9 - DNA, 4096 bp, Hepatitis B virus
caatctaagcaggctttcactttctcgccaacttacaaggcctttctgtgtaaacaatacctgaacctttaccccgttgcccggcaacggccaggtctgtgccaagtgtttgctgacgcaacccccactggctggggcttggtcatgggccatcagcgcatgcgtggaaccttttcggctcctctgccgatccatactgcggaactcctagccgcttgttttgctcgcagcaggtctggagcaaacattatcgggactgataactctgttgtcctatcccgcaaatatacatcgtttccatggctgctaggctgtgctgccaactggatcctgcgcgggacgtcctttgtttacgtcccgtcggcgctgaatcctgcggacgacccttctcggggtcgcttgggactctctcgtccccttctccgtctgccgttccgaccgaccacggggcgcacctctctttacgcggactccccgtctgtgccttctcatctgccggaccgtgtgcacttcgcttcacctctgcacgtcgcatggagaccaccgtgaacgcccaccaaatattgcccaaggtcttacataagaggactcttggactctcagcaatgtcaacgaccgaccttgaggcatacttcaaagactgtttgtttaaagactgggaggagttgggggaggagattaggttaaaggtctttgtactaggaggctgtaggcataaattggtctgcgcaccagcaccatgcaactttttcacctctgcctaatcatctcttgttcatgtcctactgttcaagcctccaagctgtgccttgggtggctttggggcatggacatcgacccttataaagaatttggagctactgtggagttactctcgtttttgccttctgacttctttccttcagtacgagatcttctagataccgcctcagctctgtatcgggaagccttagagtctcctgagcattgttcacctcaccatactgcactcaggcaagcaattctttgctggggggaactaatgactctagctacctgggtgggtgttaatttggaagatccagcgtctagagacctagtagtcagttatgtcaacactaatatgggcctaaagttcaggcaactcttgtggtttcacatttcttgtctcacttttggaagagaaacagttatagagtatttggtgtctttcggagtgtggattcgcactcctccagcttatagaccaccaaatgcccctatcctatcaacacttccggagactactgttgttagacgacgaggcaggtcccctagaagaagaactccctcgcctcgcagacgaaggtctcaatcgccgcgtcgcagaagatctcaatctcgggaatctcaatgttagtattccttggactcataaggtggggaactttactgggctttattcttctactgtacctgtctttaatcctcattggaaaacaccatcttttcctaatatacatttacaccaagacattatcaaaaaatgtgaacagtttgtaggcccactcacagttaatgagaaaagaagattgcaattgattatgcctgccaggttttatccaaaggttaccaaatatttaccattggataagggtattaaaccttattatccagaacatctagttaatcattacttccaaactagacactatttacacactctatggaaggcgggtatattatataagagagaaacaacacatagcgcctcattttgtgggtcaccatattcttgggaacaagatctacagcatggggcagaatctttccaccagcaatcctctgggattctttcccgaccaccagttggatccagccttcagagcaaacaccgcaaatccagattgggacttcaatcccaacaaggacacctggccagacgccaacaaggtaggagctggagcattcgggctgggtttcaccccaccgcacggaggccttttggggtggagccctcaggctcagggcatactacaaactttgccagcaaatccgcctcctgcctccaccaatcgccagtcaggaaggcagcctaccccgctgtctccacctttgagaaacactcatcctcaggccatgcagtggaattccacaaccttccaccaagctcctgcagatcccagagtgagaggcctgtatttccctgctggtggctccagttcaggaacagtaaaccctgttctgactactgcctctcccttatcgtcaatcttctcgaggattggggaccctgcgctgaacatggagaacatcacatcaggattcctaggaccccttctcgtgttacaggcggggtttttcttgttgacaagaatcctcacaataccgcagagtctagactcgtggtggacttctctcaattttctagggggaactaccgtgtgtcttggccaaaattcgcagtccccaacctccaatcactcaccaacctcttgtcctccaacttgtcctggttatcgctggatgtgtctgcggcgttttatcatcttcctcttcatcctgctgctatgcctcatcttcttgttggttcttctggactatcaaggtatgttgcccgtttgtcctctaattccaggatcctcaacaaccagcacgggaccatgccggacctgcatgactactgctcaaggaacctctatgtatccctcctgttgctgtaccaaaccttcggacggaaattgcacctgtattcccatcccatcatcctgggctttcggaaaattcctatgggagtgggcctcagcccgtttctcctggctcagtttactagtgccatttgttcagtggttcgtagggctttcccccactgtttggctttcagttatatggatgatgtggtattgggggccaagtctgtacagcatcttgagtccctttttaccgctgttaccaattttcttttgtctttgggtatacatttaaaccctaacaaaacaaagagatggggttactctctaaattttatgggttatgtcattggatgttatgggtccttgccacaagaacacatcatacaaaaaatcaaagaatgttttagaaaacttcctattaacaggcctattgattggaaagtatgtcaacgaattgtgggtcttttgggttttgctgccccttttacacaatgtggttatcctgcgttgatgcctttgtatgcatgtattcaatctaagcaggctttcactttctcgccaacttacaaggcctttctgtgtaaacaatacctgaacctttaccccgttgcccggcaacggccaggtctgtgccaagtgtttgctgacgcaacccccactggctggggcttggtcatgggccatcagcgcatgcgtggaaccttttcggctcctctgccgatccatactgcggaactcctagccgcttgttttgctcgcagcaggtctggagcaaacattatcgggactgataactctgttgtcctatcccgcaaatatacatcgtttccatggctgctaggctgtgctgccaactggatcctgcgcgggacgtcctttgtttacgtcccgtcggcgctgaatcctgcggacgacccttctcggggtcgcttgggactctctcgtccccttctccgtctgccgttccgaccgaccacggggcgcacctctctttacgcggactccccgtctgtgccttctcatctgccggaccgtgtgcacttcgcttcacctctgcacgtcgcatggagaccaccgtgaacgcccaccaaatattgcccaaggtcttacataagaggactcttggactctcagcaatgtcaacgaccgaccttgaggcatacttcaaagactgtttgtttaaagactgggaggagttgggggaggagattaggttaaaggtctttgtactaggaggctgtaggcataaattggtctgcgcaccagcaccatgcaactttttcacctctgcctaatcatctcttgttcatgtcctactgttcaagcctccaagctgtgccttgggtggctttggggcatggacatcgacccttataaagaatttggagctactgtggagttactctcgtttttgccttctgacttctttccttcagtacgagatc

SEQ ID NO: 10 - DNA, 8131 bp, Hepatitis B virus
acattaccctgttatccctagatgacattaccctgttatcccagatgacattaccctgttatccctagatgacattaccctgttatccctagatgacatttaccctgttatccctagatgacattaccctgttatcccagatgacattaccctgttatccctagatacattaccctgttatcccagatgacataccctgttatccctagatgacattaccctgttatcccagatgacattaccctgttatccctagatacattaccctgttatcccagatgacataccctgttatccctagatgacattaccctgttatcccagatgacattaccctgttatccctagatacattaccctgttatcccagatgacataccctgttatccctagatgacattaccctgttatcccagatgacattaccctgttatccctagatacattaccctgttatcccagatgacataccctgttatccctagatgacattaccctgttatcccagatgacattaccctgttatccctagatacattaccctgttatcccagatgacataccctgttatccctagatgacattaccctgttatcccagatgacattaccctgttatccctagatacattaccctgttatcccagatgacataccctgttatccctagatgacattaccctgttatcccagataaactcaatgatgatgatgatgatggtcgagactcagcggccgcggtgccagggcgtgcccttgggctccccgggcgcgcaatctaagcaggctttcactttctcgccaacttacaaggcctttctgtgtaaacaatacctgaacctttaccccgttgcccggcaacggccaggtctgtgccaagtgtttgctgacgcaacccccactggctggggcttggtcatgggccatcagcgcatgcgtggaaccttttcggctcctctgccgatccatactgcggaactcctagccgcttgttttgctcgcagcaggtctggagcaaacattatcgggactgataactctgttgtcctatcccgcaaatatacatcgtttccatggctgctaggctgtgctgccaactggatcctgcgcgggacgtcctttgtttacgtcccgtcggcgctgaatcctgcggacgacccttctcggggtcgcttgggactctctcgtccccttctccgtctgccgttccgaccgaccacggggcgcacctctctttacgcggactccccgtctgtgccttctcatctgccggaccgtgtgcacttcgcttcacctctgcacgtcgcatggagaccaccgtgaacgcccaccaaatattgcccaaggtcttacataagaggactcttggactctcagcaatgtcaacgaccgaccttgaggcatacttcaaagactgtttgtttaaagactgggaggagttgggggaggagattaggttaaaggtctttgtactaggaggctgtaggcataaattggtctgcgcaccagcaccatgcaactttttcacctctgcctaatcatctcttgttcatgtcctactgttcaagcctccaagctgtgccttgggtggctttggggcatggacatcgacccttataaagaatttggagctactgtggagttactctcgtttttgccttctgacttctttccttcagtacgagatcttctagataccgcctcagctctgtatcgggaagccttagagtctcctgagcattgttcacctcaccatactgcactcaggcaagcaattctttgctggggggaactaatgactctagctacctgggtgggtgttaatttggaagatccagcgtctagagacctagtagtcagttatgtcaacactaatatgggcctaaagttcaggcaactcttgtggtttcacatttcttgtctcacttttggaagagaaacagttatagagtatttggtgtctttcggagtgtggattcgcactcctccagcttatagaccaccaaatgcccctatcctatcaacacttccggagactactgttgttagacgacgaggcaggtcccctagaagaagaactccctcgcctcgcagacgaaggtctcaatcgccgcgtcgcagaagatctcaatctcgggaatctcaatgttagtattccttggactcataaggtggggaactttactgggctttattcttctactgtacctgtctttaatcctcattggaaaacaccatcttttcctaatatacatttacaccaagacattatcaaaaaatgtgaacagtttgtaggcccactcacagttaatgagaaaagaagattgcaattgattatgcctgccaggttttatccaaaggttaccaaatatttaccattggataagggtattaaaccttattatccagaacatctagttaatcattacttccaaactagacactatttacacactctatggaaggcgggtatattatataagagagaaacaacacatagcgcctcattttgtgggtcaccatattcttgggaacaagatctacagcatggggcagaatctttccaccagcaatcctctgggattctttcccgaccaccagttggatccagccttcagagcaaacaccgcaaatccagattgggacttcaatcccaacaaggacacctggccagacgccaacaaggtaggagctggagcattcgggctgggtttcaccccaccgcacggaggccttttggggtggagccctcaggctcagggcatactacaaactttgccagcaaatccgcctcctgcctccaccaatcgccagtcaggaaggcagcctaccccgctgtctccacctttgagaaacactcatcctcaggccatgcagtggaattccacaaccttccaccaaactctgcaagatcccagagtgagaggcctgtatttccctgctggtggctccagttcaggaacagtaaaccctgttctgactactgcctctcccttatcgtcaatcttctcgaggattggggaccctgcgctgaacatggagaacatcacatcaggattcctaggaccccttctcgtgttacaggcggggtttttcttgttgacaagaatcctcacaataccgcagagtctagactcgtggtggacttctctcaattttctagggggaactaccgtgtgtcttggccaaaattcgcagtccccaacctccaatcactcaccaacctcttgtcctccaacttgtcctggttatcgctggatgtgtctgcggcgttttatcatcttcctcttcatcctgctgctatgcctcatcttcttgttggttcttctggactatcaaggtatgttgcccgtttgtcctctaattccaggatcctcaacaaccagcacgggaccatgccggacctgcatgactactgctcaaggaacctctatgtatccctcctgttgctgtaccaaaccttcggacggaaattgcacctgtattcccatcccatcatcctgggctttcggaaaattcctatgggagtgggcctcagcccgtttctcctggctcagtttactagtgccatttgttcagtggttcgtagggctttcccccactgtttggctttcagttatatggatgatgtggtattgggggccaagtctgtacagcatcttgagtccctttttaccgctgttaccaattttcttttgtctttgggtatacatttaaaccctaacaaaacaaagagatggggttactctctaaattttatgggttatgtcattggatgttatgggtccttgccacaagaacacatcatacaaaaaatcaaagaatgttttagaaaacttcctattaacaggcctattgattggaaagtatgtcaacgaattgtgggtcttttgggttttgctgccccttttacacaatgtggttatcctgcgttgatgcctttgtatgcatgtattcaatctaagcaggctttcactttctcgccaacttacaaggcctttctgtgtaaacaatacctgaacctttaccccgttgcccggcaacggccaggtctgtgccaagtgtttgctgacgcaacccccactggctggggcttggtcatgggccatcagcgcatgcgtggaaccttttcggctcctctgccgatccatactgcggaactcctagccgcttgttttgctcgcagcaggtctggagcaaacattatcgggactgataactctgttgtcctatcccgcaaatatacatcgtttccatggctgctaggctgtgctgccaactggatcctgcgcgggacgtcctttgtttacgtcccgtcggcgctgaatcctgcggacgacccttctcggggtcgcttgggactctctcgtccccttctccgtctgccgttccgaccgaccacggggcgcacctctctttacgcggactccccgtctgtgccttctcatctgccggaccgtgtgcacttcgcttcacctctgcacgtcgcatggagaccaccgtgaacgcccaccaaatattgcccaaggtcttacataagaggactcttggactctcagcaatgtcaacgaccgaccttgaggcatacttcaaagactgtttgtttaaagactgggaggagttgggggaggagattaggttaaaggtctttgtactaggaggctgtaggcataaattggtctgcgcaccagcaccatgcaactttttcacctctgcctaatcatctcttgttcatgtcctactgttcaagcctccaagctgtgccttgggtggctttggggcatggacatcgacccttataaagaatttggagctactgtggagttactctcgtttttgccttctgacttctttccttcagtacgagatcccccaactggggtaacctttgagttctctcagttgggggtaatcagcatcatgatgtggtaccacatcatgatgctgattataagaatgcggccgccacactctagtggatctcgagttaataattcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgctcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgtagatgacatggagatcctgccccggcacttcgcccaatagcagccagtcccttcccgcttcagtgacaacgtcgagcacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagagcttgatcccctgcgccatcagatccttggcggcgagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatccggggtcagcaccgtttctgcggactggctttctacgtgctcgaggggggccaaacggtctccagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttttgtttatttttctaaatacattcaaatatgtatccgctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagcagatcaattcgcgcgcgaaggcgaagcggcatgcataatgtgcctgtcaaatggacgaagcagggattctgcaaaccctatgctactccgtcaagccgtcaattgtctgattcgttaccaattatgacaacttgacggctacatcattcactttttcttcacaaccggcacggaactcgctcgggctggccccggtgcattttttaaatacccgcgagaaatagagttgatcgtcaaaaccaacattgcgaccgacggtggcgataggcatccgggtggtgctcaaaagcagcttcgcctggctgatacgttggtcctcgcgccagcttaagacgctaatccctaactgctggcggaaaagatgtgacagacgcgacggcgacaagcaaacatgctgtgcgacgctggcgat

SEQ ID NO: 11 - DNA, 4180 bp, Hepatitis B virus
tgggctccccgggcgcgcaatctaagcaggctttcactttctcgccaacttacaaggcctttctgtgtaaacaatacctgaacctttaccccgttgcccggcaacggccaggtctgtgccaagtgtttgctgacgcaacccccactggctggggcttggtcatgggccatcagcgcatgcgtggaaccttttcggctcctctgccgatccatactgcggaactcctagccgcttgttttgctcgcagcaggtctggagcaaacattatcgggactgataactctgttgtcctatcccgcaaatatacatcgtttccatggctgctaggctgtgctgccaactggatcctgcgcgggacgtcctttgtttacgtcccgtcggcgctgaatcctgcggacgacccttctcggggtcgcttgggactctctcgtccccttctccgtctgccgttccgaccgaccacggggcgcacctctctttacgcggactccccgtctgtgccttctcatctgccggaccgtgtgcacttcgcttcacctctgcacgtcgcatggagaccaccgtgaacgcccaccaaatattgcccaaggtcttacataagaggactcttggactctcagcaatgtcaacgaccgaccttgaggcatacttcaaagactgtttgtttaaagactgggaggagttgggggaggagattaggttaaaggtctttgtactaggaggctgtaggcataaattggtctgcgcaccagcaccatgcaactttttcacctctgcctaatcatctcttgttcatgtcctactgttcaagcctccaagctgtgccttgggtggctttggggcatggacatcgacccttataaagaatttggagctactgtggagttactctcgtttttgccttctgacttctttccttcagtacgagatcttctagataccgcctcagctctgtatcgggaagccttagagtctcctgagcattgttcacctcaccatactgcactcaggcaagcaattctttgctggggggaactaatgactctagctacctgggtgggtgttaatttggaagatccagcgtctagagacctagtagtcagttatgtcaacactaatatgggcctaaagttcaggcaactcttgtggtttcacatttcttgtctcacttttggaagagaaacagttatagagtatttggtgtctttcggagtgtggattcgcactcctccagcttatagaccaccaaatgcccctatcctatcaacacttccggagactactgttgttagacgacgaggcaggtcccctagaagaagaactccctcgcctcgcagacgaaggtctcaatcgccgcgtcgcagaagatctcaatctcgggaatctcaatgttagtattccttggactcataaggtggggaactttactgggctttattcttctactgtacctgtctttaatcctcattggaaaacaccatcttttcctaatatacatttacaccaagacattatcaaaaaatgtgaacagtttgtaggcccactcacagttaatgagaaaagaagattgcaattgattatgcctgccaggttttatccaaaggttaccaaatatttaccattggataagggtattaaaccttattatccagaacatctagttaatcattacttccaaactagacactatttacacactctatggaaggcgggtatattatataagagagaaacaacacatagcgcctcattttgtgggtcaccatattcttgggaacaagatctacagcatggggcagaatctttccaccagcaatcctctgggattctttcccgaccaccagttggatccagccttcagagcaaacaccgcaaatccagattgggacttcaatcccaacaaggacacctggccagacgccaacaaggtaggagctggagcattcgggctgggtttcaccccaccgcacggaggccttttggggtggagccctcaggctcagggcatactacaaactttgccagcaaatccgcctcctgcctccaccaatcgccagtcaggaaggcagcctaccccgctgtctccacctttgagaaacactcatcctcaggccatgcagtggaattccacaaccttccaccaaactctgcaagatcccagagtgagaggcctgtatttccctgctggtggctccagttcaggaacagtaaaccctgttctgactactgcctctcccttatcgtcaatcttctcgaggattggggaccctgcgctgaacatggagaacatcacatcaggattcctaggaccccttctcgtgttacaggcggggtttttcttgttgacaagaatcctcacaataccgcagagtctagactcgtggtggacttctctcaattttctagggggaactaccgtgtgtcttggccaaaattcgcagtccccaacctccaatcactcaccaacctcttgtcctccaacttgtcctggttatcgctggatgtgtctgcggcgttttatcatcttcctcttcatcctgctgctatgcctcatcttcttgttggttcttctggactatcaaggtatgttgcccgtttgtcctctaattccaggatcctcaacaaccagcacgggaccatgccggacctgcatgactactgctcaaggaacctctatgtatccctcctgttgctgtaccaaaccttcggacggaaattgcacctgtattcccatcccatcatcctgggctttcggaaaattcctatgggagtgggcctcagcccgtttctcctggctcagtttactagtgccatttgttcagtggttcgtagggctttcccccactgtttggctttcagttatatggatgatgtggtattgggggccaagtctgtacagcatcttgagtccctttttaccgctgttaccaattttcttttgtctttgggtatacatttaaaccctaacaaaacaaagagatggggttactctctaaattttatgggttatgtcattggatgttatgggtccttgccacaagaacacatcatacaaaaaatcaaagaatgttttagaaaacttcctattaacaggcctattgattggaaagtatgtcaacgaattgtgggtcttttgggttttgctgccccttttacacaatgtggttatcctgcgttgatgcctttgtatgcatgtattcaatctaagcaggctttcactttctcgccaacttacaaggcctttctgtgtaaacaatacctgaacctttaccccgttgcccggcaacggccaggtctgtgccaagtgtttgctgacgcaacccccactggctggggcttggtcatgggccatcagcgcatgcgtggaaccttttcggctcctctgccgatccatactgcggaactcctagccgcttgttttgctcgcagcaggtctggagcaaacattatcgggactgataactctgttgtcctatcccgcaaatatacatcgtttccatggctgctaggctgtgctgccaactggatcctgcgcgggacgtcctttgtttacgtcccgtcggcgctgaatcctgcggacgacccttctcggggtcgcttgggactctctcgtccccttctccgtctgccgttccgaccgaccacggggcgcacctctctttacgcggactccccgtctgtgccttctcatctgccggaccgtgtgcacttcgcttcacctctgcacgtcgcatggagaccaccgtgaacgcccaccaaatattgcccaaggtcttacataagaggactcttggactctcagcaatgtcaacgaccgaccttgaggcatacttcaaagactgtttgtttaaagactgggaggagttgggggaggagattaggttaaaggtctttgtactaggaggctgtaggcataaattggtctgcgcaccagcaccatgcaactttttcacctctgcctaatcatctcttgttcatgtcctactgttcaagcctccaagctgtgccttgggtggctttggggcatggacatcgacccttataaagaatttggagctactgtggagttactctcgtttttgccttctgacttctttccttcagtacgagatcccccaactggggtaacctttgagttctctcagttgggggtaatcagcatcatgatgtggtaccacat

SEQ ID NO: 12 - DNA, 3211 bp, Hepatitis B virus
cccgggcgcgaccatggggcagaatctttccaccagcaatcctctgggattctttcccgaccaccagttggatccagccttcagagcaaacaccgcaaatccagattgggacttcaatcccaacaaggacacctggccagacgccaacaaggtaggagctggagcattcgggctgggtttcaccccaccgcacggaggccttttggggtggagccctcaggctcagggcatactacaaactttgccagcaaatccgcctcctgcctccaccaatcgccagtcaggaaggcagcctaccccgctgtctccacctttgagaaacactcatcctcaggccatgcagtggaattccacaaccttccaccaaactctgcaagatcccagagtgagaggcctgtatttccctgctggtggctccagttcaggaacagtaaaccctgttctgactactgcctctcccttatcgtcaatcttctcgaggattggggaccctgcgctgaacatggagaacatcacatcaggattcctaggaccccttctcgtgttacaggcggggtttttcttgttgacaagaatcctcacaataccgcagagtctagactcgtggtggacttctctcaattttctagggggaactaccgtgtgtcttggccaaaattcgcagtccccaacctccaatcactcaccaacctcttgtcctccaacttgtcctggttatcgctggatgtgtctgcggcgttttatcatcttcctcttcatcctgctgctatgcctcatcttcttgttggttcttctggactatcaaggtatgttgcccgtttgtcctctaattccaggatcctcaacaaccagcacgggaccatgccggacctgcatgactactgctcaaggaacctctatgtatccctcctgttgctgtaccaaaccttcggacggaaattgcacctgtattcccatcccatcatcctgggctttcggaaaattcctatgggagtgggcctcagcccgtttctcctggctcagtttactagtgccatttgttcagtggttcgtagggctttcccccactgtttggctttcagttatatggatgatgtggtattgggggccaagtctgtacagcatcttgagtccctttttaccgctgttaccaattttcttttgtctttgggtatacatttaaaccctaacaaaacaaagagatggggttactctctaaattttatgggttatgtcattggatgttatgggtccttgccacaagaacacatcatacaaaaaatcaaagaatgttttagaaaacttcctattaacaggcctattgattggaaagtatgtcaacgaattgtgggtcttttgggttttgctgccccttttacacaatgtggttatcctgcgttgatgcctttgtatgcatgtattcaatctaagcaggctttcactttctcgccaacttacaaggcctttctgtgtaaacaatacctgaacctttaccccgttgcccggcaacggccaggtctgtgccaagtgtttgctgacgcaacccccactggctggggcttggtcatgggccatcagcgcatgcgtggaaccttttcggctcctctgccgatccatactgcggaactcctagccgcttgttttgctcgcagcaggtctggagcaaacattatcgggactgataactctgttgtcctatcccgcaaatatacatcgtttccatggctgctaggctgtgctgccaactggatcctgcgcgggacgtcctttgtttacgtcccgtcggcgctgaatcctgcggacgacccttctcggggtcgcttgggactctctcgtccccttctccgtctgccgttccgaccgaccacggggcgcacctctctttacgcggactccccgtctgtgccttctcatctgccggaccgtgtgcacttcgcttcacctctgcacgtcgcatggagaccaccgtgaacgcccaccaaatattgcccaaggtcttacataagaggactcttggactctcagcaatgtcaacgaccgaccttgaggcatacttcaaagactgtttgtttaaagactgggaggagttgggggaggagattaggttaaaggtctttgtactaggaggctgtaggcataaattggtctgcgcaccagcaccatgcaactttttcacctctgcctaatcatctcttgttcatgtcctactgttcaagcctccaagctgtgccttgggtggctttggggcatggacatcgacccttataaagaatttggagctactgtggagttactctcgtttttgccttctgacttctttccttcagtacgagatcttctagataccgcctcagctctgtatcgggaagccttagagtctcctgagcattgttcacctcaccatactgcactcaggcaagcaattctttgctggggggaactaatgactctagctacctgggtgggtgttaatttggaagatccagcgtctagagacctagtagtcagttatgtcaacactaatatgggcctaaagttcaggcaactcttgtggtttcacatttcttgtctcacttttggaagagaaacagttatagagtatttggtgtctttcggagtgtggattcgcactcctccagcttatagaccaccaaatgcccctatcctatcaacacttccggagactactgttgttagacgacgaggcaggtcccctagaagaagaactccctcgcctcgcagacgaaggtctcaatcgccgcgtcgcagaagatctcaatctcgggaatctcaatgttagtattccttggactcataaggtggggaactttactgggctttattcttctactgtacctgtctttaatcctcattggaaaacaccatcttttcctaatatacatttacaccaagacattatcaaaaaatgtgaacagtttgtaggcccactcacagttaatgagaaaagaagattgcaattgattatgcctgccaggttttatccaaaggttaccaaatatttaccattggataagggtattaaaccttattatccagaacatctagttaatcattacttccaaactagacactatttacacactctatggaaggcgggtatattatataagagagaaacaacacatagcgcctcattttgtgggtcaccatattcttgggaacaagatctacagcccccaactggggtacc

SEQ ID NO: 13 - DNA, 5832 bp, Hepatitis B virus
tcttctctcatccgccaaaacagccaagctggagaccgtttgacattaccctgttatccctagatacattaccctgttatcccagatgacataccctgttatccctagatgacattaccctgttatcccagatgacattaccctgttatccctagatacattaccctgttatcccagatgacataccctgttatccctagatgacattaccctgttatcccagatgacattaccctgttatccctagatacattaccctgttatcccagatgacataccctgttatccctagatgacattaccctgttatcccagatgacattaccctgttatccctagatacattaccctgttatcccagatgacataccctgttatccctagatgacattaccctgttatcccagatgacattaccctgttatccctagatacattaccctgttatcccagatgacataccctgttatccctagatgacattaccctgttatcccagatgacattaccctgttatccctagatacattaccctgttatcccagatgacataccctgttatccctagatgacattaccctgttatcccagatgacattaccctgttatccctagatacattaccctgttatcccagatgacataccctgttatccctagatgacattaccctgttatcccagatgacattaccctgttatccctagatacattaccctgttatcccagatgacataccctgttatccctagatgacattaccctgttatcccagataaactcaatgatgatgatgatgatggtcgagactcagcggccgcggtgccagggcgtgcccttgggctccccgggcgcgactagtgaattgatactagtattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtttatataagcagagctcgtttagtgaaccgtcagatcgcctggagacgccatccacgctgttttgacctccatagaagattctagagtcgacgcggccgcggatccatttaaattcgaattcgctagctcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggtacctttaagaccaatgacttacaaggcagctgtagatcttagccactttttaaaagaaaaggggggactggaagggctaattcactcccaacgaagataagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagacttttgcagatcgacccatgggggcccgccccaactggggtaacctttgagttctctcagttgggggtaatcagcatcatgatgtggtaccacatcatgatgctgattataagaatgcggccgccacactctagtggatctcgagttaataattcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgctcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgtagatgacatggagatcctgccccggcacttcgcccaatagcagccagtcccttcccgcttcagtgacaacgtcgagcacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagagcttgatcccctgcgccatcagatccttggcggcgagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatccggggtcagcaccgtttctgcggactggctttctacgtgctcgaggggggccaaacggtctccagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttttgtttatttttctaaatacattcaaatatgtatccgctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagcagatcaattcgcgcgcgaaggcgaagcggcatgcataatgtgcctgtcaaatggacgaagcagggattctgcaaaccctatgctactccgtcaagccgtcaattgtctgattcgttaccaattatgacaacttgacggctacatcattcactttttcttcacaaccggcacggaactcgctcgggctggccccggtgcattttttaaatacccgcgagaaatagagttgatcgtcaaaaccaacattgcgaccgacggtggcgataggcatccgggtggtgctcaaaagcagcttcgcctggctgatacgttggtcctcgcgccagcttaagacgctaatccctaactgctggcggaaaagatgtgacagacgcgacggcgacaagcaaacatgctgtgcgacgctggcgat

SEQ ID NO: 14 - DNA, 3182 bp, Hepatitis B virus
ttccacaaccttccaccaagctcctgcagatcccagagtgagaggcctgtatttccctgctggtggctccagttcaggaacagtaaaccctgttctgactactgcctctcccttatcgtcaatcttctcgaggattggggaccctgcgctgaacatggagaacatcacatcaggattcctaggaccccttctcgtgttacaggcggggtttttcttgttgacaagaatcctcacaataccgcagagtctagactcgtggtggacttctctcaattttctagggggaactaccgtgtgtcttggccaaaattcgcagtccccaacctccaatcactcaccaacctcttgtcctccaacttgtcctggttatcgctggatgtgtctgcggcgttttatcatcttcctcttcatcctgctgctatgcctcatcttcttgttggttcttctggactatcaaggtatgttgcccgtttgtcctctaattccaggatcctcaacaaccagcacgggaccatgccggacctgcatgactactgctcaaggaacctctatgtatccctcctgttgctgtaccaaaccttcggacggaaattgcacctgtattcccatcccatcatcctgggctttcggaaaattcctatgggagtgggcctcagcccgtttctcctggctcagtttactagtgccatttgttcagtggttcgtagggctttcccccactgtttggctttcagttatatggatgatgtggtattgggggccaagtctgtacagcatcttgagtccctttttaccgctgttaccaattttcttttgtctttgggtatacatttaaaccctaacaaaacaaagagatggggttactctctaaattttatgggttatgtcattggatgttatgggtccttgccacaagaacacatcatacaaaaaatcaaagaatgttttagaaaacttcctattaacaggcctattgattggaaagtatgtcaacgaattgtgggtcttttgggttttgctgccccttttacacaatgtggttatcctgcgttgatgcctttgtatgcatgtattcaatctaagcaggctttcactttctcgccaacttacaaggcctttctgtgtaaacaatacctgaacctttaccccgttgcccggcaacggccaggtctgtgccaagtgtttgctgacgcaacccccactggctggggcttggtcatgggccatcagcgcatgcgtggaaccttttcggctcctctgccgatccatactgcggaactcctagccgcttgttttgctcgcagcaggtctggagcaaacattatcgggactgataactctgttgtcctatcccgcaaatatacatcgtttccatggctgctaggctgtgctgccaactggatcctgcgcgggacgtcctttgtttacgtcccgtcggcgctgaatcctgcggacgacccttctcggggtcgcttgggactctctcgtccccttctccgtctgccgttccgaccgaccacggggcgcacctctctttacgcggactccccgtctgtgccttctcatctgccggaccgtgtgcacttcgcttcacctctgcacgtcgcatggagaccaccgtgaacgcccaccaaatattgcccaaggtcttacataagaggactcttggactctcagcaatgtcaacgaccgaccttgaggcatacttcaaagactgtttgtttaaagactgggaggagttgggggaggagattaggttaaaggtctttgtactaggaggctgtaggcataaattggtctgcgcaccagcaccatgcaactttttcacctctgcctaatcatctcttgttcatgtcctactgttcaagcctccaagctgtgccttgggtggctttggggcgtggacatcgacccttataaagaatttggagctactgtggagttactctcgtttttgccttctgacttctttccttcagtacgagatcttctagataccgcctcagctctgtatcgggaagccttagagtctcctgagcattgttcacctcaccatactgcactcaggcaagcaattctttgctggggggaactaatgactctagctacctgggtgggtgttaatttggaagatccagcgtctagagacctagtagtcagttatgtcaacactaatatgggcctaaagttcaggcaactcttgtggtttcacatttcttgtctcacttttggaagagaaacagttatagagtatttggtgtctttcggagtgtggattcgcactcctccagcttatagaccaccaaatgcccctatcctatcaacacttccggagactactgttgttagacgacgaggcaggtcccctagaagaagaactccctcgcctcgcagacgaaggtctcaatcgccgcgtcgcagaagatctcaatctcgggaatctcaatgttagtattccttggactcataaggtggggaactttactgggctttattcttctactgtacctgtctttaatcctcattggaaaacaccatcttttcctaatatacatttacaccaagacattatcaaaaaatgtgaacagtttgtaggcccactcacagttaatgagaaaagaagattgcaattgattatgcctgccaggttttatccaaaggttaccaaatatttaccattggataagggtattaaaccttattatccagaacatctagttaatcattacttccaaactagacactatttacacactctatggaaggcgggtatattatataagagagaaacaacacatagcgcctcattttgtgggtcaccatattcttgggaacaagatctacagcatggggcagaatctttccaccagcaatcctctgggattctttcccgaccaccagttggatccagccttcagagcaaacaccgcaaatccagattgggacttcaatcccaacaaggacacctggccagacgccaacaaggtaggagctggagcattcgggctgggtttcaccccaccgcacggaggccttttggggtggagccctcaggctcagggcatactacaaactttgccagcaaatccgcctcctgcctccaccaatcgccagtcaggaaggcagcctaccccgctgtctccacctttgagaaacactcatcctcaggccatgcagtggaa

SEQ ID NO: 15 - DNA, 3221 bp, Hepatitis B virus
cccgggcgcgaccatggggcagaatctttccaccagcaatcctctgggattctttcccgaccaccagttggatccagccttcagagcaaacaccgcaaatccagattgggacttcaatcccaacaaggacacctggccagacgccaacaaggtaggagctggagcattcgggctgggtttcaccccaccgcacggaggccttttggggtggagccctcaggctcagggcatactacaaactttgccagcaaatccgcctcctgcctccaccaatcgccagtcaggaaggcagcctaccccgctgtctccacctttgagaaacactcatcctcaggccatgcagtggaattccacaaccttccaccaaactctgcaagatcccagagtgagaggcctgtatttccctgctggtggctccagttcaggaacagtaaaccctgttctgactactgcctctcccttatcgtcaatcttctcgaggattggggaccctgcgctgaacatggagaacatcacatcaggattcctaggaccccttctcgtgttacaggcggggtttttcttgttgacaagaatcctcacaataccgcagagtctagactcgtggtggacttctctcaattttctagggggaactaccgtgtgtcttggccaaaattcgcagtccccaacctccaatcactcaccaacctcttgtcctccaacttgtcctggttatcgctggatgtgtctgcggcgttttatcatcttcctcttcatcctgctgctatgcctcatcttcttgttggttcttctggactatcaaggtatgttgcccgtttgtcctctaattccaggatcctcaacaaccagcacgggaccatgccggacctgcatgactactgctcaaggaacctctatgtatccctcctgttgctgtaccaaaccttcggacggaaattgcacctgtattcccatcccatcatcctgggctttcggaaaattcctatgggagtgggcctcagcccgtttctcctggctcagtttactagtgccatttgttcagtggttcgtagggctttcccccactgtttggctttcagttatatggatgatgtggtattgggggccaagtctgtacagcatcttgagtccctttttaccgctgttaccaattttcttttgtctttgggtatacatttaaaccctaacaaaacaaagagatggggttactctctaaattttatgggttatgtcattggatgttatgggtccttgccacaagaacacatcatacaaaaaatcaaagaatgttttagaaaacttcctattaacaggcctattgattggaaagtatgtcaacgaattgtgggtcttttgggttttgctgccccttttacacaatgtggttatcctgcgttgatgcctttgtatgcatgtattcaatctaagcaggctttcactttctcgccaacttacaaggcctttctgtgtaaacaatacctgaacctttaccccgttgcccggcaacggccaggtctgtgccaagtgtttgctgacgcaacccccactggctggggcttggtcatgggccatcagcgcatgcgtggaaccttttcggctcctctgccgatccatactgcggaactcctagccgcttgttttgctcgcagcaggtctggagcaaacattatcgggactgataactctgttgtcctatcccgcaaatatacatcgtttccatggctgctaggctgtgctgccaactggatcctgcgcgggacgtcctttgtttacgtcccgtcggcgctgaatcctgcggacgacccttctcggggtcgcttgggactctctcgtccccttctccgtctgccgttccgaccgaccacggggcgcacctctctttacgcggactccccgtctgtgccttctcatctgccggaccgtgtgcacttcgcttcacctctgcacgtcgcatggagaccaccgtgaacgcccaccaaatattgcccaaggtcttacataagaggactcttggactctcagcaatgtcaacgaccgaccttgaggcatacttcaaagactgtttgtttaaagactgggaggagttgggggaggagattaggttaaaggtctttgtactaggaggctgtaggcataaattggtctgcgcaccagcaccatgcaactttttcacctctgcctaatcatctcttgttcatgtcctactgttcaagcctccaagctgtgccttgggtggctttggggcgtggacatcgacccttataaagaatttggagctactgtggagttactctcgtttttgccttctgacttctttccttcagtacgagatcttctagataccgcctcagctctgtatcgggaagccttagagtctcctgagcattgttcacctcaccatactgcactcaggcaagcaattctttgctggggggaactaatgactctagctacctgggtgggtgttaatttggaagatccagcgtctagagacctagtagtcagttatgtcaacactaatatgggcctaaagttcaggcaactcttgtggtttcacatttcttgtctcacttttggaagagaaacagttatagagtatttggtgtctttcggagtgtggattcgcactcctccagcttatagaccaccaaatgcccctatcctatcaacacttccggagactactgttgttagacgacgaggcaggtcccctagaagaagaactccctcgcctcgcagacgaaggtctcaatcgccgcgtcgcagaagatctcaatctcgggaatctcaatgttagtattccttggactcataaggtggggaactttactgggctttattcttctactgtacctgtctttaatcctcattggaaaacaccatcttttcctaatatacatttacaccaagacattatcaaaaaatgtgaacagtttgtaggcccactcacagttaatgagaaaagaagattgcaattgattatgcctgccaggttttatccaaaggttaccaaatatttaccattggataagggtattaaaccttattatccagaacatctagttaatcattacttccaaactagacactatttacacactctatggaaggcgggtatattatataagagagaaacaacacatagcgcctcattttgtgggtcaccatattcttgggaacaagatctacagcccccaactggggtaacctttgggctc

SEQ ID NO: 16 - DNA, 3221 bp, Hepatitis B virus
cccgggcgcgaccatggggcagaatctttccaccagcaatcctctgggattctttcccgaccaccagttggatccagccttcagagcaaacaccgcaaatccagattgggacttcaatcccaacaaggacacctggccagacgccaacaaggtaggagctggagcattcgggctgggtttcaccccaccgcacggaggccttttggggtggagccctcaggctcagggcatactacaaactttgccagcaaatccgcctcctgcctccaccaatcgccagtcaggaaggcagcctaccccgctgtctccacctttgagaaacactcatcctcaggccatgcagtggaattccacaaccttccaccaaactctgcaagatcccagagtgagaggcctgtatttccctgctggtggctccagttcaggaacagtaaaccctgttctgactactgcctctcccttatcgtcaatcttctcgaggattggggaccctgcgctgaacatggagaacatcacatcaggattcctaggaccccttctcgtgttacaggcggggtttttcttgttgacaagaatcctcacaataccgcagagtctagactcgtggtggacttctctcaattttctagggggaactaccgtgtgtcttggccaaaattcgcagtccccaacctccaatcactcaccaacctcttgtcctccaacttgtcctggttatcgctggatgtgtctgcggcgttttatcatcttcctcttcatcctgctgctatgcctcatcttcttgttggttcttctggactatcaaggtatgttgcccgtttgtcctctaattccaggatcctcaacaaccagcacgggaccatgccggacctgcatgactactgctcaaggaacctctatgtatccctcctgttgctgtaccaaaccttcggacggaaattgcacctgtattcccatcccatcatcctgggctttcggaaaattcctatgggagtgggcctcagcccgtttctcctggctcagtttactagtgccatttgttcagtggttcgtagggctttcccccactgtttggctttcagttatatggatgatgtggtattgggggccaagtctgtacagcatcttgagtccctttttaccgctgttaccaattttcttttgtctttgggtatacatttaaaccctaacaaaacaaagagatggggttactctctaaattttatgggttatgtcattggatgttatgggtccttgccacaagaacacatcatacaaaaaatcaaagaatgttttagaaaacttcctattaacaggcctattgattggaaagtatgtcaacgaattgtgggtcttttgggttttgctgccccttttacacaatgtggttatcctgcgttgatgcctttgtatgcatgtattcaatctaagcaggctttcactttctcgccaacttacaaggcctttctgtgtaaacaatacctgaacctttaccccgttgcccggcaacggccaggtctgtgccaagtgtttgctgacgcaacccccactggctggggcttggtcatgggccatcagcgcatgcgtggaaccttttcggctcctctgccgatccatactgcggaactcctagccgcttgttttgctcgcagcaggtctggagcaaacattatcgggactgataactctgttgtcctatcccgcaaatatacatcgtttccatggctgctaggctgtgctgccaactggatcctgcgcgggacgtcctttgtttacgtcccgtcggcgctgaatcctgcggacgacccttctcggggtcgcttgggactctctcgtccccttctccgtctgccgttccgaccgaccacggggcgcacctctctttacgcggactccccgtctgtgccttctcatctgccggaccgtgtgcacttcgcttcacctctgcacgtcgcatggagaccaccgtgaacgcccaccaaatattgcccaaggtcttacataagaggactcttggactctcagcaatgtcaacgaccgaccttgaggcatacttcaaagactgtttgtttaaagactgggaggagttgggggaggagattaggttaaaggtctttgtactaggaggctgtaggcataaattggtctgcgcaccagcaccatgcaactttttcacctctgcctaatcatctcttgttcatgtcctactgttcaagcctccaagctgtgccttgggtggctttggggcatggacatcgacccttataaagaatttggagctactgtggagttactctcgtttttgccttctgacttctttccttcagtacgagatcttctagataccgcctcagctctgtatcgggaagccttagagtctcctgagcattgttcacctcaccatactgcactcaggcaagcaattctttgctggggggaactaatgactctagctacctgggtgggtgttaatttggaagatccagcgtctagagacctagtagtcagttatgtcaacactaatatgggcctaaagttcaggcaactcttgtggtttcacatttcttgtctcacttttggaagagaaacagttatagagtatttggtgtctttcggagtgtggattcgcactcctccagcttatagaccaccaaacgcccctatcctatcaacacttccggagactactgttgttagacgacgaggcaggtcccctagaagaagaactccctcgcctcgcagacgaaggtctcaatcgccgcgtcgcagaagatctcaatctcgggaatctcaatgttagtattccttggactcataaggtggggaactttactgggctttattcttctactgtacctgtctttaatcctcattggaaaacaccatcttttcctaatatacatttacaccaagacattatcaaaaaatgtgaacagtttgtaggcccactcacagttaatgagaaaagaagattgcaattgattatgcctgccaggttttatccaaaggttaccaaatatttaccattggataagggtattaaaccttattatccagaacatctagttaatcattacttccaaactagacactatttacacactctatggaaggcgggtatattatataagagagaaacaacacatagcgcctcattttgtgggtcaccatattcttgggaacaagatctacagcccccaactggggtaacctttgggctc

SEQ ID NO: 17 - DNA, 3221 bp, Hepatitis B virus
cccgggcgcgaccatggggcagaatctttccaccagcaatcctctgggattctttcccgaccaccagttggatccagccttcagagcaaacaccgcaaatccagattgggacttcaatcccaacaaggacacctggccagacgccaacaaggtaggagctggagcattcgggctgggtttcaccccaccgcacggaggccttttggggtggagccctcaggctcagggcatactacaaactttgccagcaaatccgcctcctgcctccaccaatcgccagtcaggaaggcagcctaccccgctgtctccacctttgagaaacactcatcctcaggccatgcagtggaattccacaaccttccaccaaactctgcaagatcccagagtgagaggcctgtatttccctgctggtggctccagttcaggaacagtaaaccctgttctgactactgcctctcccttatcgtcaatcttctcgaggattggggaccctgcgctgaacatggagaacatcacatcaggattcctaggaccccttctcgtgttacaggcggggtttttcttgttgacaagaatcctcacaataccgcagagtctagactcgtggtggacttctctcaattttctagggggaactaccgtgtgtcttggccaaaattcgcagtccccaacctccaatcactcaccaacctcttgtcctccaacttgtcctggttatcgctggatgtgtctgcggcgttttatcatcttcctcttcatcctgctgctatgcctcatcttcttgttggttcttctggactatcaaggtatgttgcccgtttgtcctctaattccaggatcctcaacaaccagcacgggaccatgccggacctgcatgactactgctcaaggaacctctatgtatccctcctgttgctgtaccaaaccttcggacggaaattgcacctgtattcccatcccatcatcctgggctttcggaaaattcctatgggagtgggcctcagcccgtttctcctggctcagtttactagtgccatttgttcagtggttcgtagggctttcccccactgtttggctttcagttatatggatgatgtggtattgggggccaagtctgtacagcatcttgagtccctttttaccgctgttaccaattttcttttgtctttgggtatacatttaaaccctaacaaaacaaagagatggggttactctctaaattttatgggttatgtcattggatgttatgggtccttgccacaagaacacatcatacaaaaaatcaaagaatgttttagaaaacttcctattaacaggcctattgattggaaagtatgtcaacgaattgtgggtcttttgggttttgctgccccttttacacaatgtggttatcctgcgttgatgcctttgtatgcatgtattcaatctaagcaggctttcactttctcgccaacttacaaggcctttctgtgtaaacaatacctgaacctttaccccgttgcccggcaacggccaggtctgtgccaagtgtttgctgacgcaacccccactggctggggcttggtcatgggccatcagcgcatgcgtggaaccttttcggctcctctgccgatccatactgcggaactcctagccgcttgttttgctcgcagcaggtctggagcaaacattatcgggactgataactctgttgtcctatcccgcaaatatacatcgtttccatggctgctaggctgtgctgccaactggatcctgcgcgggacgtcctttgtttacgtcccgtcggcgctgaatcctgcggacgacccttctcggggtcgcttgggactctctcgtccccttctccgtctgccgttccgaccgaccacggggcgcacctctctttacgcggactccccgtctgtgccttctcatctgccggaccgtgtgcacttcgcttcacctctgcacgtcgcatggagaccaccgtgaacgcccaccaaatattgcccaaggtcttacataagaggactcttggactctcagcaatgtcaacgaccgaccttgaggcatacttcaaagactgtttgtttaaagactgggaggagttgggggaggagattaggttaaaggtctttgtactaggaggctgtaggcataaattggtctgcgcaccagcaccatgcaactttttcacctctgcctaatcatctcttgttcatgtcctactgttcaagcctccaagctgtgccttgggtggctttggggcatggacatcgacccttataaagaatttggagctactgtggagttactctcgtttttgccttctgacttctttccttcagtacgagatcttctagataccgcctcagctctgtatcgggaagccttagagtctcctgagcattgttcacctcaccatactgcactcaggcaagcaattctttgctggggggaactaatgactctagctacctgggtgggtgttaatttggaagatccagcgtctagagacctagtagtcagttatgtcaacactaatatgggcctaaagttcaggcaactcttgtggtttcacatttcttgtctcacttttggaagagaaacagttatagagtatttggtgtctttcggagtgtggattcgcactcctccagcttatagaccaccaaatgcccctatcctatcaacacttccggagactactgttgttagacgacgaggcaggtcccctagaagaagaactccctcgcctcgcagacgaaggtctcaatcgccgcgtcgcagaagatctcaatctcgggaatctcaatgttagtattccttggactcataaggtggggaactttactgggcttgattcttctactgtacctgtctttaatcctcattggaaaacaccatcttttcctaatatacatttacaccaagacattatcaaaaaatgtgaacagtttgtaggcccactcacagttaatgagaaaagaagattgcaattgattatgcctgccaggttttatccaaaggttaccaaatatttaccattggataagggtattaaaccttattatccagaacatctagttaatcattacttccaaactagacactatttacacactctatggaaggcgggtatattatataagagagaaacaacacatagcgcctcattttgtgggtcaccatattcttgggaacaagatctacagcccccaactggggtaacctttgggctc

SEQ ID NO: 18 - DNA, 3221 bp, Hepatitis B virus
cccgggcgcgaccatggggcagaatctttccaccagcaatcctctgggattctttcccgaccaccagttggatccagccttcagagcaaacaccgcaaatccagattgggacttcaatcccaacaaggacacctggccagacgccaacaaggtaggagctggagcattcgggctgggtttcaccccaccgcacggaggccttttggggtggagccctcaggctcagggcatactacaaactttgccagcaaatccgcctcctgcctccaccaatcgccagtcaggaaggcagcctaccccgctgtctccacctttgagaaacactcatcctcaggccatgcagtggaattccacaaccttccaccaaactctgcaagatcccagagtgagaggcctgtatttccctgctggtggctccagttaaggaacagtaaaccctgttctgactactgcctctcccttatcgtcaatcttctcgaggattggggaccctgcgctgaacatggagaacatcacataaggattcctaggaccccttctcgtgttacaggcggggtttttcttgttgacaagaatcctcacaataccgcagagtctagactcgtggtggacttctctcaattttctagggggaactaccgtgtgtcttggccaaaattcgcagtccccaacctccaatcactcaccaacctcttgtcctccaacttgtcctggttatcgctggatgtgtctgcggcgttttatcatcttcctcttcatcctgctgctatgcctcatcttcttgttggttcttctggactatcaaggtatgttgcccgtttgtcctctaattccaggatcctcaacaaccagcacgggaccatgccggacctgcatgactactgctcaaggaacctctatgtatccctcctgttgctgtaccaaaccttcggacggaaattgcacctgtattcccatcccatcatcctgggctttcggaaaattcctatgggagtgggcctcagcccgtttctcctggctcagtttactagtgccatttgttcagtggttcgtagggctttcccccactgtttggctttcagttatatggatgatgtggtattgggggccaagtctgtacagcatcttgagtccctttttaccgctgttaccaattttcttttgtctttgggtatacatttaaaccctaacaaaacaaagagatggggttactctctaaattttatgggttatgtcattggatgttatgggtccttgccacaagaacacatcatacaaaaaatcaaagaatgttttagaaaacttcctattaacaggcctattgattggaaagtatgtcaacgaattgtgggtcttttgggttttgctgccccttttacacaatgtggttatcctgcgttgatgcctttgtatgcatgtattcaatctaagcaggctttcactttctcgccaacttacaaggcctttctgtgtaaacaatacctgaacctttaccccgttgcccggcaacggccaggtctgtgccaagtgtttgctgacgcaacccccactggctggggcttggtcatgggccatcagcgcatgcgtggaaccttttcggctcctctgccgatccatactgcggaactcctagccgcttgttttgctcgcagcaggtctggagcaaacattatcgggactgataactctgttgtcctatcccgcaaatatacatcgtttccatggctgctaggctgtgctgccaactggatcctgcgcgggacgtcctttgtttacgtcccgtcggcgctgaatcctgcggacgacccttctcggggtcgcttgggactctctcgtccccttctccgtctgccgttccgaccgaccacggggcgcacctctctttacgcggactccccgtctgtgccttctcatctgccggaccgtgtgcacttcgcttcacctctgcacgtcgcatggagaccaccgtgaacgcccaccaaatattgcccaaggtcttacataagaggactcttggactctcagcaatgtcaacgaccgaccttgaggcatacttcaaagactgtttgtttaaagactgggaggagttgggggaggagattaggttaaaggtctttgtactaggaggctgtaggcataaattggtctgcgcaccagcaccatgcaactttttcacctctgcctaatcatctcttgttcatgtcctactgttcaagcctccaagctgtgccttgggtggctttggggcatggacatcgacccttataaagaatttggagctactgtggagttactctcgtttttgccttctgacttctttccttcagtacgagatcttctagataccgcctcagctctgtatcgggaagccttagagtctcctgagcattgttcacctcaccatactgcactcaggcaagcaattctttgctggggggaactaatgactctagctacctgggtgggtgttaatttggaagatccagcgtctagagacctagtagtcagttatgtcaacactaatatgggcctaaagttcaggcaactcttgtggtttcacatttcttgtctcacttttggaagagaaacagttatagagtatttggtgtctttcggagtgtggattcgcactcctccagcttatagaccaccaaatgcccctatcctatcaacacttccggagactactgttgttagacgacgaggcaggtcccctagaagaagaactccctcgcctcgcagacgaaggtctcaatcgccgcgtcgcagaagatctcaatctcgggaatctcaatgttagtattccttggactcataaggtggggaactttactgggctttattcttctactgtacctgtctttaatcctcattggaaaacaccatcttttcctaatatacatttacaccaagacattatcaaaaaatgtgaacagtttgtaggcccactcacagttaatgagaaaagaagattgcaattgattatgcctgccaggttttatccaaaggttaccaaatatttaccattggataagggtattaaaccttattatccagaacatctagttaatcattacttccaaactagacactatttacacactctatggaaggcgggtatattatataagagagaaacaacacatagcgcctcattttgtgggtcaccatattcttgggaacaagatctacagcccccaactggggtaacctttgggctc

SEQ ID NO: 19 - DNA, 3221 bp, Hepatitis B virus
cccgggcgcgaccatggggcagaatctttccaccagcaatcctctgggattctttcccgaccaccagttggatccagccttcagagcaaacaccgcaaatccagattgggacttcaatcccaacaaggacacctggccagacgccaacaaggtaggagctggagcattcgggctgggtttcaccccaccgcacggaggccttttggggtggagccctcaggctcagggcatactacaaactttgccagcaaatccgcctcctgcctccaccaatcgccagtcaggaaggcagcctaccccgctgtctccacctttgagaaacactcatcctcaggccatgcagtggaattccacaaccttccaccaaactctgcaagatcccagagtgagaggcctgtatttccctgctggtggctccagttcaggaacagtaaaccctgttctgactactgcctctcccttatcgtcaatcttctcgaggattggggaccctgcgctgaacatggagaacatcacatcaggattcctaggaccccttctcgtgttacaggcggggtttttcttgttgacaagaatcctcacaataccgcagagtctagactcgtggtggacttctctcaattttctagggggaactaccgtgtgtcttggccaaaattcgcagtccccaacctccaatcactcaccaacctcttgtcctccaacttgtcctggttatcgctggatgtgtctgcggcgttttatcatcttcctcttcatcctgctgctatgcctcatcttcttgttggttcttctggactatcaaggtatgttgcccgtttgtcctctaattccaggatcctcaacaaccagcacgggaccatgccggacctgcatgactactgctcaaggaacctctatgtatccctcctgttgctgtaccaaaccttcggacggaaattgcacctgtattcccatcccatcatcctgggctttcggaaaattcctatgggagtgggcctcagcccgtttctcctggctcagtttactagtgccatttgttcagtggttcgtagggctttcccccactgtttggctttcagttatatggatgatgtggtattgggggccaagtctgtacagcatcttgagtccctttttaccgctgttaccaattttcttttgtctttgggtatacatttaaaccctaacaaaacaaagagatggggttactctctaaattttatgggttatgtcattggatgttatgggtccttgccacaagaacacatcatacaaaaaatcaaagaatgttttagaaaacttcctattaacaggcctattgattggaaagtatgtcaacgaattgtgggtcttttgggttttgctgccccttttacacaatgtggttatcctgcgttgatgcctttgtatgcatgtattcaatctaagcaggctttcactttctcgccaacttacaaggcctttctgtgtaaacaatacctgaacctttaccccgttgcccggcaacggccaggtctgtgccaagtgtttgctgacgcaacccccactggctggggcttggtcatgggccatcagcgcatgcgtggaaccttttcggctcctctgccgatccatactgcggaactcctagccgcttgttttgctcgcagcaggtctggagcaaacattatcgggactgataactctgttgtcctatcccgcaaatatacatcgtttccatggctgctaggctgtgctgccaactggatcctgcgcgggacgtcctttgtttacgtcccgtcggcgctgaatcctgcggacgacccttctcggggtcgcttgggactctctcgtccccttctccgtctgccgttccgaccgaccacggggcgcacctctctttacgcggactccccgtctgtgccttctcatctgccggaccgtgtgcacttcgcttcacctctgcacgtcgcatggagaccaccgtgaacgcccaccaaatattgcccaaggtcttacataagaggactcttggactctcagcaatgtcaacgaccgaccttgaggcatacttcaaagactgtttgtttaaagactgggaggagttgggggaggagattaggttaaaggtctttgtactaggaggctgtaggcataaattggtctgcgcaccagcaccatgcaactttttcacctctgcctaatcatctcttgttcatgtcctactgttcaagcctccaagctgtgccttgggtggctttagggcatggacatcgacccttataaagaatttggagctactgtggagttactctcgtttttgccttctgacttctttccttcagtacgagatcttctagataccgcctcagctctgtatcgggaagccttagagtctcctgagcattgttcacctcaccatactgcactcaggcaagcaattctttgctggggggaactaatgactctagctacctgggtgggtgttaatttggaagatccagcgtctagagacctagtagtcagttatgtcaacactaatatgggcctaaagttcaggcaactcttgtggtttcacatttcttgtctcacttttggaagagaaacagttatagagtatttggtgtctttcggagtgtggattcgcactcctccagcttatagaccaccaaatgcccctatcctatcaacacttccggagactactgttgttagacgacgaggcaggtcccctagaagaagaactccctcgcctcgcagacgaaggtctcaatcgccgcgtcgcagaagatctcaatctcgggaatctcaatgttagtattccttggactcataaggtggggaactttactgggctttattcttctactgtacctgtctttaatcctcattggaaaacaccatcttttcctaatatacatttacaccaagacattatcaaaaaatgtgaacagtttgtaggcccactcacagttaatgagaaaagaagattgcaattgattatgcctgccaggttttatccaaaggttaccaaatatttaccattggataagggtattaaaccttattatccagaacatctagttaatcattacttccaaactagacactatttacacactctatggaaggcgggtatattatataagagagaaacaacacatagcgcctcattttgtgggtcaccatattcttgggaacaagatctacagcccccaactggggtaacctttgggctc

SEQ ID NO: 20 - DNA, 3221 bp, Hepatitis B virus
cccgggcgcgaccatggggcagaatctttccaccagcaatcctctgggattctttcccgaccaccagttggatccagccttcagagcaaacaccgcaaatccagattgggacttcaatcccaacaaggacacctggccagacgccaacaaggtaggagctggagcattcgggctgggtttcaccccaccgcacggaggccttttggggtggagccctcaggctcagggcatactacaaactttgccagcaaatccgcctcctgcctccaccaatcgccagtcaggaaggcagcctaccccgctgtctccacctttgagaaacactcatcctcaggccatgcagtggaattccacaaccttccaccaaactctgcaagatcccagagtgagaggcctgtatttccctgctggtggctccagttcaggaacagtaaaccctgttctgactactgcctctcccttatcgtcaatcttctcgaggattggggaccctgcgctgaacatggagaacatcacatcaggattcctaggaccccttctcgtgttacaggcggggtttttcttgttgacaagaatcctcacaataccgcagagtctagactcgtggtggacttctctcaattttctagggggaactaccgtgtgtcttggccaaaattcgcagtccccaacctccaatcactcaccaacctcttgtcctccaacttgtcctggttatcgctggatgtgtctgcggcgttttatcatcttcctcttcatcctgctgctatgcctcatcttcttgttggttcttctggactatcaaggtatgttgcccgtttgtcctctaattccaggatcctcaacaaccagcacgggaccatgccggacctgcatgactactgctcaaggaacctctatgtatccctcctgttgctgtaccaaaccttcggacggaaattgcacctgtattcccatcccatcatcctgggctttcggaaaattcctatgggagtgggcctcagcccgtttctcctggctcagtttactagtgccatttgttcagtggttcgtagggctttcccccactgtttggctttcagttatatggatgatgtggtattgggggccaagtctgtacagcatcttgagtccctttttaccgctgttaccaattttcttttgtctttgggtatacatttaaaccctaacaaaacaaagagatggggttactctctaaattttatgggttatgtcattggatgttatgggtccttgccacaagaacacatcatacaaaaaatcaaagaatgttttagaaaacttcctattaacaggcctattgattggaaagtatgtcaacgaattgtgggtcttttgggttttgctgccccttttacacaatgtggttatcctgcgttgatgcctttgtatgcatgtattcaatctaagcaggctttcactttctcgccaacttacaaggcctttctgtgtaaacaatacctgaacctttaccccgttgcccggcaacggccaggtctgtgccaagtgtttgctgacgcaacccccactggctggggcttggtcatgggccatcagcgcatgcgtggaaccttttcggctcctctgccgatccatactgcggaactcctagccgcttgttttgctcgcagcaggtctggagcaaacattatcgggactgataactctgttgtcctatcccgcaaatatacatcgtttccatggctgctaggctgtgctgctaactggatcctgcgcgggacgtcctttgtttacgtcccgtcggcgctgaatcctgcggacgacccttctcggggtcgcttgggactctctcgtccccttctccgtctgccgttccgaccgaccacggggcgcacctctctttacgcggactccccgtctgtgccttctcatctgccggaccgtgtgcacttcgcttcacctctgcacgtcgcatggagaccaccgtgaacgcccaccaaatattgcccaaggtcttacataagaggactcttggactctcagcaatgtcaacgaccgaccttgaggcatacttcaaagactgtttgtttaaagactgggaggagttgggggaggagattaggttaaaggtctttgtactaggaggctgtaggcataaattggtctgcgcaccagcaccatgcaactttttcacctctgcctaatcatctcttgttcatgtcctactgttcaagcctccaagctgtgccttgggtggctttggggcatggacatcgacccttataaagaatttggagctactgtggagttactctcgtttttgccttctgacttctttccttcagtacgagatcttctagataccgcctcagctctgtatcgggaagccttagagtctcctgagcattgttcacctcaccatactgcactcaggcaagcaattctttgctggggggaactaatgactctagctacctgggtgggtgttaatttggaagatccagcgtctagagacctagtagtcagttatgtcaacactaatatgggcctaaagttcaggcaactcttgtggtttcacatttcttgtctcacttttggaagagaaacagttatagagtatttggtgtctttcggagtgtggattcgcactcctccagcttatagaccaccaaatgcccctatcctatcaacacttccggagactactgttgttagacgacgaggcaggtcccctagaagaagaactccctcgcctcgcagacgaaggtctcaatcgccgcgtcgcagaagatctcaatctcgggaatctcaatgttagtattccttggactcataaggtggggaactttactgggctttattcttctactgtacctgtctttaatcctcattggaaaacaccatcttttcctaatatacatttacaccaagacattatcaaaaaatgtgaacagtttgtaggcccactcacagttaatgagaaaagaagattgcaattgattatgcctgccaggttttatccaaaggttaccaaatatttaccattggataagggtattaaaccttattatccagaacatctagttaatcattacttccaaactagacactatttacacactctatggaaggcgggtatattatataagagagaaacaacacatagcgcctcattttgtgggtcaccatattcttgggaacaagatctacagcccccaactggggtaacctttgggctc

SEQ ID NO: 21 - DNA, 3221 bp, Hepatitis B virus
cccgggcgcgaccatggggcagaatctttccaccagcaatcctctgggattctttcccgaccaccagttggatccagccttcagagcaaacaccgcaaatccagattgggacttcaatcccaacaaggacacctggccagacgccaacaaggtaggagctggagcattcgggctgggtttcaccccaccgcacggaggccttttggggtggagccctcaggctcagggcatactacaaactttgccagcaaatccgcctcctgcctccaccaatcgccagtcaggaaggcagcctaccccgctgtctccacctttgagaaacactcatcctcaggccatgcagtggaattccacaaccttccaccaaactctgcaagatcccagagtgagaggcctgtatttccctgctggtggctccagttcaggaacagtaaaccctgttctgactactgcctctcccttatcgtcaatcttctcgaggattggggaccctgcgctgaacatggagaacatcacatcaggattcctaggaccccttctcgtgttacaggcggggtttttcttgttgacaagaatcctcacaataccgcagagtctagactcgtggtggacttctctcaattttctagggggaactaccgtgtgtcttggccaaaattcgcagtccccaacctccaatcactcaccaacctcttgtcctccaacttgtcctggttatcgctggatgtgtctgcggcgttttatcatcttcctcttcatcctgctgctatgcctcatcttcttgttggttcttctggactatcaaggtatgttgcccgtttgtcctctaattccaggatcctcaacaaccagcacgggaccatgccggacctgcatgactactgctcaaggaacctctatgtatccctcctgttgctgtaccaaaccttcggacggaaattgcacctgtattcccatcccatcatcctgggctttcggaaaattcctatgggagtgggcctcagcccgtttctcctggctcagtttactagtgccatttgttcagtggttcgtagggctttcccccactgtttggctttcagttatatggatgatgtggtattgggggccaagtctgtacagcatcttgagtccctttttaccgctgttaccaattttcttttgtctttgggtatacatttaaaccctaacaaaacaaagagatggggttactctctaaattttatgggttatgtcattggatgttatgggtccttgccacaagaacacatcatacaaaaaatcaaagaatgttttagaaaacttcctattaacaggcctattgattggaaagtatgtcaacgaattgtgggtcttttgggttttgctgccccttttacacaatgtggttatcctgcgttgatgcctttgtatgcatgtattcaatctaagcaggctttcactttctcgccaacttacaaggcctttctgtgtaaacaatacctgaacctttaccccgttgcccggcaacggccaggtctgtgccaagtgtttgctgacgcaacccccactggctggggcttggtcatgggccatcagcgcatgcgtggaaccttttcggctcctctgccgatccatactgcggaactcctagccgcttgttttgctcgcagcaggtctggagcaaacattatcgggactgataactctgttgtcctatcccgcaaatatacatcgtttccatggctgctaggctgtgctgccaactggatcctgcgcgggacgtcctttgtttacgtcccgtcggcgctgaatcctgcggacgacccttctcggggtcgcttgggactctctcgtccccttctccgtctgccgttccgaccgaccacggggcgcacctctctttacgcggactccccgtctgtgccttctcatctgccggaccgtgtgcacttcgcttcacctctgcacgtcgcatggagaccaccgtgaacgcccaccaaatattgcccaaggtcttacataaggagactcttggactctcagcaatgtcaacgaccgaccttgaggcatacttcaaagactgtttgtttaaagactgggaggagttgggggaggagattaggttaaaggtctttgtactaggaggctgtaggcataaattggtctgcgcaccagcaccatgcaactttttcacctctgcctaatcatctcttgttcatgtcctactgttcaagcctccaagctgtgccttgggtggctttggggcatggacatcgacccttataaagaatttggagctactgtggagttactctcgtttttgccttctgacttctttccttcagtacgagatcttctagataccgcctcagctctgtatcgggaagccttagagtctcctgagcattgttcacctcaccatactgcactcaggcaagcaattctttgctggggggaactaatgactctagctacctgggtgggtgttaatttggaagatccagcgtctagagacctagtagtcagttatgtcaacactaatatgggcctaaagttcaggcaactcttgtggtttcacatttcttgtctcacttttggaagagaaacagttatagagtatttggtgtctttcggagtgtggattcgcactcctccagcttatagaccaccaaatgcccctatcctatcaacacttccggagactactgttgttagacgacgaggcaggtcccctagaagaagaactccctcgcctcgcagacgaaggtctcaatcgccgcgtcgcagaagatctcaatctcgggaatctcaatgttagtattccttggactcataaggtggggaactttactgggctttattcttctactgtacctgtctttaatcctcattggaaaacaccatcttttcctaatatacatttacaccaagacattatcaaaaaatgtgaacagtttgtaggcccactcacagttaatgagaaaagaagattgcaattgattatgcctgccaggttttatccaaaggttaccaaatatttaccattggataagggtattaaaccttattatccagaacatctagttaatcattacttccaaactagacactatttacacactctatggaaggcgggtatattatataagagagaaacaacacatagcgcctcattttgtgggtcaccatattcttgggaacaagatctacagcccccaactggggtaacctttgggctc

SEQ ID NO: 22 - DNA, 3291 bp, Hepatitis B virus
cccgggcgcgaccatgggaggttggtcttccaaacctcgaaaaggcatggggacaaatctttctgtccccaatcccctgggattcttccccgatcatcagttggaccctgcattcaaagccaactcagaaaatccagattgggacctcaacccgcacaaggacaactggccggacgccaacaaggtgggagtgggagcattcgggccagggttcacccctccccatgggggactgttggggtggagccctcaggctcagggcctactcacaactgtgccagcagctcctcctcctgcctccaccaatcggcagttaggaaggcagcctactcccttatctccacctctaagggacactcatcctcaggccatgcagtggaactccaccactttccaccaaactcttcaagatcccagagtcagggccctgtactttcctgctggtggctccagttcaggaacagtgagccctgctcaaaatactgtctctgccatatcgtcaatcttatcgaaaactggggaccctgtaccgaacatggagaacatcgcatcaggactcctaggacccctgctcgtgttacaggcggggtttttcttgttgacaaaaatcctcacaataccacagagtctagactcgtggtggacttctctcaattttctagggggaacacccgtgtgtcttggccaaaattcgcagtcccaaatctccagtcactcaccaacctgttgtcctccaatttgtcctggttatcgctggatgtatctgcggcgttttatcatattcctctgcatcctgctgctatgcctcatcttcttgttggttcttctggactatcaaggtatgttgcccgtttgtcctctaattccaggatcatcaacaaccagcaccggaccatgcaaaacctgcacgactcctgctcaaggaacctctatgtttccctcatgttgctgtacaaaacctacggacggaaactgcacctgtattcccatcccatcatcttgggctttcgcaaaattcctatgggagtgggcctcagtccgtttctcttggctcagtttactagtgccatttgttcagtggttcgtagggctttcccccactgtctggctttcagttatatggatgatttggttttgggggccaagtctgtacaacatcttgagtccctttatgccgctgttaccaattttcttttgtctttgggtatacatttaaaccctcacaaaacaaaaagatggggatattcccttaactttatgggatatgtaattgggagttggggcacattgccacaggaacatattgtacaaaaaatcaaaatatgttttaggaaacttcctgtaaacaggcctattgattggaaagtctgtcaacgaattgtgggtcttttggggtttgccgcccctttcacgcaatgtggatatcctgctttaatgcctttatatgcatgtatacaagcaaaacaggcttttattttctcgccaacttacaaggcctttctgagtaaacagtatttgaacctttaccccgttgctcggcaacggcctggtctgtgccaagtgtttgctgacgcaacccccactggttggggcttggccataggccatcagcgcatgcgtggcacctttgtgtctcctctgccgatccatactgcggaactcctagccgcttgttttgctcgcagcaggtctggggcaaaactcatcgggactgacaattctgtcgtgctctcccgcaagtatacatcatttccatggctgctaggctgtgctgccaactggatcctgcgcgggacgtcctttgtttacgtcccgtcggcgctgaatcccgcggacgacccctcccggggccgcttggggctctaccgcccgcttctccgcctgttgtaccgaccgaccacggggcgcacctctctttacgcggactccccgtctgtgccttctcatctgccggaccgtgtgcacttcgcttcacctctgcacgtcgcatggaaaccaccgtgaacgcccacaggaacctgcccaaggtcttgcataagaggactcttggactttcagcaatgtcaacgaccgaccttgaggcatacttcaaagactgtgtgtttactgagtgggaggagttgggggaggaggttaggttaatgatctttgtactaggaggctgtaggcataaattggtgtgttcaccagcaccatgcaactttttcacctctgcctaatcatctcatgttcatgtcctactgttcaagcctccaagctgtgccttgggtggctttggggcatggacattgacccgtataaagaatttggagcttctgtggagttactctcttttttgccttctgacttctttccttctattcgagatctcctcgacaccgcctctgctctgtatcgggaggccttagagtctccggaacattgttcacctcaccatacggcactcaggcaagctattctgtgttggggtgagttaatgaatctagccacctgggtgggaagtaatttggaagatccagcatccagggaattagtagtcagctatgtcaacgttaatatgggcctaaaaatcagacaactattgtggtttcacatttcctgtcttacttttgggagagaaactgttcttgaatatttggtgtcttttggagtgtggattcgcactcctcccgcatatagaccgccaaatgcccctatcttatcaacacttccggaaactactgttgttagacgaagaggcaggtcccctagaagaagaactccctcgcctcgcagacgaaggtctcaatcgccgcgtcgcagaagatctcaatctcgggaatctcaatgttagtattccttggacacacaaggtgggaaactttacggggctttattcttctacggtaccttgctttaatcctaaatggcaaactccttcttttcctgacattcatttgcaggaggacattgttgatagatgtaagcaatttgtggggccccttacagtaaatgaaaacaggagacttaaattaattatgcctgctaggttttatcccaatgttactaaatatttgcccttagataaagggatcaaaccgtattatccagagtatgtagttaatcattacttccagacgcgacattatttacacactctttggaaggcggggatcttatataaaagagagtccacacgtagcgcctcattttgcgggtcaccatattcttgggaacaagatctacagcccccaactggggtaacctttgagttctctcagttgggggtaatcagcatcatgatgtggtacc

SEQ ID NO: 23 - DNA, 3291 bp, Hepatitis B virus
cccgggcgcgaccatgggaggttggtcttccaaacctcgaaaaggcatggggacaaatctttctgtccccaatcccctgggattcttccccgatcatcagttggaccctgcattcaaagccaactcagaaaatccagattgggacctcaacccgcacaaggacaactggccggacgccaacaaggtgggagtgggagcattcgggccagggttcacccctccccatgggggactgttggggtggagccctcaggctcagggcctactcacaactgtgccagcagctcctcctcctgcctccaccaatcggcagtcaggaaggcagcctactcccttatctccacctctaagggacactcatcctcaggccatgcagtggaactccaccactttccaccaaactcttcaagatcccagagtcagggccctgtactttcctgctggtggctccagttcaggaacagtgagccctgctcagaatactgtctctgccatatcgtcaatcttatcgaagactggggaccctgtaccgaacatggagaacatcgcatcaggactcctaggacccctgctcgtgttacaggcggggtttttcttgttgacaaaaatcctcacaataccacagagtctagactcgtggtggacttctctcaattttctagggggaacacccgtgtgtcttggccaaaattcgcagtcccaaatctccagtcactcaccaacctgttgtcctccaatttgtcctggttatcgctggatgtgtctgcggcgttttatcatcttcctctgcatcctgctgctatgcctcatcttcttgttggttcttctggactatcaaggtatgttgcccgtttgtcctctaattccaggatcatcaacaaccagcaccggaccatgcaaaacctgcacaactcctgctcaaggaacctctatgtttccctcatgttgctgtacaaaacctacggacggaaactgcacctgtattcccatcccatcatcttgggctttcgcaaaatacctatgggagtgggcctcagtccgtttctcttggctcagtttactagtgccatttgttcagtggttcgtagggctttcccccactgtctggctttcagttatatggatgatgtggttttgggggccaagtctgtacaacatcttgagtccctttatgccgctgttaccaattttcttttgtctttgggtatacatttaaaccctcacaaaacaaaaagatggggatattcccttaacttcatgggatatgtaattgggagttggggcacattgccacaggaacatattgtacaaaaaatcaaaatgtgttttaggaaacttcctgtaaacaggcctattgattggaaagtatgtcaacgaattgtgggtcttttggggtttgccgcccctttcacgcaatgtggatatcctgctttaatgcctttatatgcatgtatacaagcaaaacaggcttttactttctcgccaacttacaaggcctttctaagtaaacagtatctgaacctttaccccgttgctcggcaacggcctggtctgtgccaagtgtttgctgacgcaacccccactggttggggcttggccataggccatcagcgcatgcgtggaacctttgtgtctcctctgccgatccatactgcggaactcctagccgcttgttttgctcgcagcaggtctggggcaaaactcatcgggactgacaattctgtcgtgctctcccgcaagtatacatcatttccatggctgctaggctgtgctgccaactggatcctgcgcgggacgtcctttgtttacgtcccgtcggcgctgaatcccgcggacgacccctcccggggccgcttggggctctaccgcccgcttctccgcctgttgtaccgaccgaccacggggcgcacctctctttacgcggactccccgtctgtgccttctcatctgccggaccgtgtgcacttcgcttcacctctgcacgtcgcatggagaccaccgtgaacgcccacaggaacctgcccaaggtcttgcataagaggactcttggactttcagcaatgtcaacgaccgaccttgaggcatacttcaaagactgtgtgtttactgagtgggaggagttgggggaggaggttaggttaaaggtctttgtactaggaggctgtaggcataaattggtgtgttcaccagcaccatgcaactttttcacctctgcctaatcatctcatgttcatgtcctactgttcaagcctccaagctgtgccttgggtggctttggggcatggacattgacccgtataaagaatttggagcttctgtggagttactctcttttttgccttctgacttctttccttctattcgagatctcctcgacaccgcctctgctctgtatcgggaggccttagagtctccggaacattgttcacctcaccatacggcactcaggcaagctattctgtgttggggtgagttgatgaatctagccacctgggtgggaagtaatttggaagatccagcatccagggaattagtagtcagctatgtcaacgttaatatgggcctaaaaatcagacaactattgtggtttcacatttcctgtcttacttttgggagagaaactgttcttgaatatttggtgtcttttggagtgtggattcgcactcctcctgcatatagaccaccaaatgcccctatcttatcaacacttccggaaactactgttgttagacgaagaggcaggtcccctagaagaagaactccctcgcctcgcagacgaaggtctcaatcgccgcgtcgcagaagatctcaatctcgggaatctcaatgttagtattccttggacacataaggtgggaaactttacggggctttattcttctacggtaccttgctttaatcctaaatggcaaactccttcttttcctgacattcatttgcaggaggacattgttgatagatgtaagcaatttgtggggccccttacagtaaatgaaaacaggagactaaaattaattatgcctgctaggttttatcccaatgttactaaatatttgcccttagataaagggatcaaaccgtattatccagagtatgtagttaatcattacttccagacgcgacattatttacacactctttggaaggcggggatcttatataaaagagagtccacacgtagcgcctcattttgcgggtcaccatattcttgggaacaagatctacagcccccaactggggtaacctttgagttctctcagttgggggtaatcagcatcatgatgtggtacc